(19) United States
(12) Patent Application Publication
(10) Pub. No.: US 2025/0307717 A1
(43) Pub. Date: Oct. 2, 2025

(54) HYBRID CHEMICAL COMPUTER

(71) Applicant: THE UNIVERSITY COURT OF THE UNIVERSITY OF GLASGOW, Glasgow, Strathclyde (GB)

(72) Inventors: Leroy CRONIN, Glasgow, Strathclyde (GB); Abhishek SHARMA, Glasgow, Strathclyde (GB)

(21) Appl. No.: 18/848,875

(22) PCT Filed: Mar. 21, 2023
(86) PCT No.: PCT/EP2023/057243

§ 371 Date: Sep. 19, 2024

(30) Foreign Application Priority Data
Mar. 21, 2022 (GB) 2203955.6

(51) Int. Cl. G06N 99/00; G16C 20/10

(52) U.S. Cl. CPC G06N 99/007; G16C 20/10

(57) ABSTRACT

The present invention provides a chemical computer comprising a matrix, an input device and an analytical device. The matrix comprises a plurality of interconnected reaction spaces holding a reaction mixture, and the reaction spaces are interconnected by fluid channels. The input device is for independently addressing each of a plurality of reaction spaces within the matrix, and to independently address one or more fluid channels. The analytical device has a sensor to analyse a reaction characteristic of a reaction mixture in one or more reaction spaces. Also provided the use of the chemical computer as such, and methods of computing using the chemical computer, where such methods comprise the step of addressing the reaction spaces, optionally addressing the fluid channels, and analysing a reaction characteristic of a reaction mixture in a reaction space.

### RELATED APPLICATION

The present application claim priority to, and the benefit of 2203955.6 filed on 21 Mar. 2022 (21 Mar. 2022), the contents of which are hereby incorporated by reference in their entirety.

### FIELD OF THE INVENTION

The present invention provides a non-digital programmable chemical computer for use in computation, as well as methods of non-digital computation using the chemical computer.

### BACKGROUND

Over the last 50 years computers have become ubiquitous, essential for many aspects of modern life. During this period their processing power has increased manifold, but the paradigm used has remained the same keeping the processor and memory separate (Toffoli), and using binary state electronic switches. Systems based upon quantum effects promise to solve problems intractable for conventional computers (Zhu et al.), but they are yet to reach their full potential. However, nature exploits the parallelism of collective networks by developing systems able to process information despite large amounts of noise (Watts et al.; Deco et al.).

Quantum computing algorithms have shown the potential to solve problems that are intractable on classic computing machines but still suffer from scalability issues due to physical and technological limitations (Bennet et al.). Various approaches to what has become known as unconventional computation are being developed based on mapping computational logic to various physical phenomena (Fang et al.). These tend to emulate transistor-based logic gates and other circuit components into the physical domain using architectures based on Boolean circuits or discovered using artificial intelligence (Katsikis et al.; Lin et al.). One notable exception has been the area of DNA computing which has been developed to exploit the sequence recognition and programmable architecture of DNA to do digital computations, perform pattern recognition and implement some algorithms for various classes of problems (Qian et al.; Woods et al.). Other classical computational architectures which utilize the true nature of physical phenomena include reaction-diffusion28 and neuromorphic computers (Torrejon et al.).

Numerous approaches towards chemical computation have been proposed and implemented utilizing reaction-diffusion systems and their emerging spatiotemporal patterns (Adamatzky et al.). These architectures and their algorithms have been designed to solve a specific set of abstract mathematical problems (Kuhnert et al.; Parrilla-Gutierrez et al.; Steinbock et al.; Tsompanas et al.; Hadaeghi et al.). The challenge still remains to control the nonlinear spatiotemporal evolution of chemical waves in a controlled and programmable way to demonstrate computation via chemical decision-making and algorithmic control, to build a computing machine based on a chemical substrate.

One of the present inventors has described in WO 2020/058516 a chemical computer for use as a logic gate and as a data store. The chemical computer comprises a matrix, an input device and an analytical device. The matrix comprises a plurality of interconnected reaction spaces holding a reaction mixture, the input device is provided to independently address each of a plurality of reaction spaces within the matrix; and the analytical device has a sensor to analyse a reaction characteristic of a reaction mixture in one or more reaction spaces

There is a desire for alternative systems of computing using alternative strategies to process store and retrieve information that are low power, increase the number of computational states, and allow non-silicon-based substrate for computation.

### SUMMARY OF THE INVENTION

The present inventors have established that chemical reactions may be exploited for use in information processing, and more particularly, chemical reactivity may be reliably and repeatedly used in methods of non-digital computation.

In a general aspect, the present invention provides a chemical computer, which utilizes individually addressable and addressed, but fully interconnected, reaction spaces within a matrix. The matrix holds a reaction mixture which is capable of real time operation. The interconnections between the reaction spaces are gates, and one or more gates is individually addressable and addressed.

The addressable reaction spaces may be regarded as excitation units, and addressable interconnections may be regarded as coupling gates. The positive or negative coupling between neighbouring reaction spaces, with control from the coupling gates, allows for the matrix to act as a component of a real computer.

The matrix of the chemical computer is an electronic programmable chemical array. This is a hybrid electronic-chemical processor architecture that uses configurable arrays of chemical reactions as a computational substrate. The architecture comprises an addressable network, exemplified in the present case as an addressable network of chemical oscillators based upon the Belousov-Zhabotinsky (BZ) reaction partitioned in the matrix.

Each cell is electronically addressable, and the hybrid computation combines nearest neighbour interactions in the chemical substrate with digital logic with a vast space of input states. The architecture gives the flexibility to switch between deterministic and probabilistic computational domains.

There is an efficiency gain when the computation is distributed between the digital and the chemical domains, for the implementation of efficient algorithms which exploit non-deterministic computational architectures. The computer of the invention allows for hybrid electronic-chemical computation exemplified by one- and two-dimensional Chemical Cellular Automata (CCA) and by solving combinatorial optimization problems.

The chemical computer provides for non-digital (that is non-Boolean logic) operations within its architecture. The computational architecture processes information in an electronically programmable chemical medium. Here, computations are performed within, and take advantage of, the chemical substrate where the input-output (I/O) from the device is achieved via a digital-electronic interface.

In a first aspect of the invention there is provided a chemical computer comprising a matrix, an input device and an analytical device, wherein:
the matrix comprises a plurality of interconnected reaction spaces holding a reaction mixture, wherein the reaction spaces are interconnected by fluid channels;
the input device is provided to independently address each of a plurality of reaction spaces within the matrix, and to independently address one or more fluid channels; and
the analytical device has a sensor to analyse a reaction characteristic of a reaction mixture in one or more reaction spaces.

A fluid channel provides a fluid channel between neighbouring reaction spaces. Typically, a reaction space is connected to at least two neighbouring reaction spaces via separate fluid channels. Reaction spaces in the matrix may connect to more than two reactions spaces, such as three or four reactions spaces, where the connections are made via separate fluid channels. The fluid channels provide an interface between neighbouring reaction spaces.

The fluid channels are addressable, and addressing a fluid channel controls chemical interactions between neighbouring reaction spaces in the matrix. In particular, neighbouring reaction spaces can interact with each other through localized mass transfer and mixing at the interface. Thus, addressing a channel allows for control of coupling between reaction spaces, and more broadly across the matrix.

The reaction mixture may be a reaction mixture for a chemical oscillator reaction.

The chemical oscillator reaction may be selected from the group consisting of a Belousov-Zhabotinsky (BZ) reaction, a Briggs-Rauscher reaction and a Bray-Liebhafsky reaction, such as a Belousov-Zhabotinsky (BZ) reaction.

The reaction mixture may be a reaction mixture having a colour change in its reaction, and here the analytical device has an optical sensor to analyse the colour change in one or more reaction spaces. An oscillation reaction may therefore be oscillations between species of different colour.

The input device is for independently providing an input to each of a plurality of reaction spaces within the matrix. Additionally, the input device is for independently providing an input to one or more fluid channels, such as each of a plurality of channels.

The input may be selected from the group consisting of a mechanical force, an optical input, an electrical input, a sonic input, a magnetic input and a thermal input. Typically, the input device is for independently providing a mechanical force to each of a plurality of reaction spaces within the matrix. Here, the input device is also suitable for independently providing a mechanical force to one or more fluid channels within the matrix.

The invention also provides a computer which comprises the chemical computer of the invention.

The present invention also provides a method for the operation of a chemical computer, for example for use as a logic gate or for use in data storage and retrieval, the method comprising the steps of:
(i) providing the chemical computer of the invention;
(ii) addressing a first and/or a second reaction space in the matrix using the input device, optionally together with addressing a fluid channel connecting the first and/or second reaction spaces;
(iii) analysing a reaction characteristic of a third reaction space using the analytical device;
(iv) determining a logic result based on the addressing step and the analysis step.

These and other aspects and embodiments of the invention are discussed in further detail below.

### SUMMARY OF THE FIGURES

FIG. 1 shows a conceptual and schematic design of a hybrid computer, where (A) is a diagram of a chemical computation architecture comprising of an array of chemical transistors. The top figure shows hybrid digital-chemical information processing within single and coupled hybrid logical units. The bottom figure demonstrates a single processing step of the hybrid state machine with information looping between digital and chemical domains. Here, D, P, C and T represent digital, physical, chemical and transfer state machines; (B) shows a pictorial representation of how the information propagates together with local information processing in the chemical array. The weakly connected network in the chemical medium provides the global clock (SYNC signal) on which the local interactions process information and perform computation; and (C and D) are schematic diagrams of the two-dimensional BZ architecture showing how local cellular vortices interact by tuning the speed of the interfacial stirrers. The amplitude of the oscillations is controlled by the speed of the cell stirrers and can be used to define discrete states for information processing. Due to the well-defined periodic behaviour in the weak coupling limit, these oscillations can also be used to create a global clocking SYNC signal for decision making.

FIG. 2 shows a schematic design and physical implementation of the experimental platform, where (A) is a diagram of the automated closed-loop experimental setup showing 3D printed reactor, motor control, imaging unit and supporting electronics; (B) is an exploded view of the 3D printed reactor array with stirrers mapped with motor array and fluidic connections; (C) shows the complete experimental setup inside a light controlled acrylic housing with fluidic connections from the pump control unit; (D) is a closeup view of the 3D printed reactor array with emerging BZ oscillation patterns; and (E) is a closeup view of the motor array with magnets connected to motor shafts controlling stirrer actuation.

FIG. 3 shows chemical clocking in one-dimensional BZ experiments, where (A) shows the BZ oscillations recorded for 60 mins in a continuously stirred single reactor; (B) shows the BZ oscillations recorded in a single cell with LOW/HIGH states with programmable switching based on global clocking signal, (C) shows the deviations between the peak oscillations with time (peak number) between different interconnected cells with global clocking; (D) shows chemical oscillations and discrete chemical states by applying CNN for a single BZ cell vs. time for pure clocking tests (top) and cellular automata tests (down); (E) shows two different examples of cell and global clock interpreted from CNN output; and (F) and (G) are the temporal snapshots of the actual experiments in a one-dimensional cell array demonstrating clocking wave without and with chemical mapped chemical states.

FIG. 4 shows an implementation of one-dimensional Chemical Cellular Automata and State Space expansion, where (A, B) show representations of hybrid electronic-chemical state machine working in deterministic and probabilistic computational modes. (black arrow: deterministic, red arrow: probabilistic); (C) shows an implementation of elementary CA (rule 30) in deterministic mode showing the one-to-one mapping between digital and chemical states, Top: rule table and observed chemical states (light blue: 0, blue: 1), bottom: observed oscillations in the experiment with CNN states in the background; (D) shows an implementation of non-standard CA rule 30 in the probabilistic model showing deviation from one-to-one mapping. Top right and bottom show examples of deviations from one-to-one mapping in single and multiple cells between digital (DS) and chemical states (CS); and (E) shows at Top: Global chemical states and input states vs size of the experimental array (n×n) with a different number of detection levels and PWM controls. Middle: Pictorial representation of 1D-CCA rule (rule 30-7) table for two chemical states (0: Black and 1: White). The coloured box in the centre represents the PWM state for the cell stirrer (Red/Green) and the neighbouring boxes represent the PWM state of the interfacial stirrers (Red/Blue). Bottom: Simulated 1D-CCA variants of rule 30 with different interfacial inputs based on phenomenological model.

FIG. 5 shows an experimental demonstration of 2D CCA where (A) are snapshots of the two-dimensional experimental platform showing propagation and replication events starting with a single Chemical Entity; (B) is a schematic conceptual description of closed-loop probabilistic logic for emergent dynamics of Chemits; (C & D) provide a detailed description of experimental Chemits showing propagation and replication dynamics using four-state PWM logic on cell stirrers and two-state on interfacial stirrers together with experimental controls. Top Left: shows four different PWM states {0,1,2,3} applied on cell stirrers, Top right: shown PWM levels only applied to interfacial stirrers connecting the Chemit, Bottom left: shows emerging chemical states from oscillatory patterns of three discrete CNN states, Bottom right: shows the interpreted Chemits from the combination of chemical (CS) and stirrer (PWM) states; (E) shows the population dynamics of Chemits on two-dimensional experimental step (7×7 cells) vs oscillatory time steps; and (F) show the number of high chemical states vs. time from which Chemits are derived based on PWM logic.

FIG. 6 shows simulations of 2D-CCA using a simplified phenomenological model where (A) shows basic construct of the experimental Chemit and demonstrates propagation (1), replication (2), competition (3-5), and random selection (6) between multiple events in a 5×5 array using the 2D-CCA state machine as a proof-of-principle; (B) shows snapshots of simulations of 2D-CCA using the phenomenological model at different time steps t=0 and t=15000 with different initial conditions (IC: the initial number of Chemits) on 100×100 grid; (C) shows at top left: the time evolution of the average number of Chemits with different initial conditions which converge at a steady-state depending on the available resource space (each simulation for different initial conditions were performed 25 times); and shows at top right a phase space of stabilized population of Chemits after 10000 simulation steps with two different probabilities (prob1: Q₂ and prob2: Q₄) in a phenomenological model. Bottom Left: show population dynamics of Chemits with different domain sizes. Bottom Right: show population dynamics of Chemits at different random events describing the equivalent of “energy input” with the same initial conditions.

FIG. 7 shows a demonstration of chemical computation in the chemical array, where (A) is a representation of the mapping of an abstract problem on the chemical array and lookup tables for chemical states interactions (light blue: CS₀ and dark blue CS₁); (B) shows the mapping of a 4-number partition problem with showing variable coupling and its representation on the chemical array platform with multiple instantiations of spins (auxiliary cells); (C) is a flow chart describing hybrid electronic-chemical logic for solving quadratic optimization problems using a chemical array; (D) is an energy minimization of four-number partitioning solved using a hybrid probabilistic algorithm (Top: first approach, Bottom: second approach): (E) is a pictorial representation of chemical states from an experiment on energy minimization for a 4-number partition problem (light blue: CS₀, dark blue: CS₁); and (F) shows the success probability of solving an 8-number partition problem (S={1,3,4,9,3,5,3,6}) vs. initial configuration index at different deterministic index values.

FIG. 8 shows a schematic of the experimental setup used in the worked examples of the automated experimental platform of the present case, with chemical inputs, experimental setup and digital domain. The chemical inputs create the BZ mixture from the stock solutions using pump control. The experimental setup runs the experiment with motor control and imaging. The digital domain runs real-time data analysis and state machines for hybrid electronic-chemical information processing.

FIG. 9 shows the intensity of oscillations recorded over the seven cells of a one-dimensional platform with cell 1, cell 3, cell 5 and cell 7 were activated with periodic actuation from cell stirrers. The experiments demonstrate that the actuation of cell stirrers is localized to the specific cell.

FIG. 10 is a schematic showing hydrodynamic coupling between two neighbouring cells with inactive and active interfacial cells.

FIG. 11 shows snapshots at different times for ink flows between two neighbouring cells in an active interface, demonstrating symmetric coupling between the two cells.

FIG. 12 shows snapshots at different times for ink flows between two neighbouring cells in an active interface, demonstrating asymmetric coupling between the two cells.

FIG. 13 shows snapshots of the two-dimensional platform at different times for ink flows between cells, showing the coupling between the centre and left cell by activating the interfacial stirrer between them. No mass transfer was observed between other neighbours.

FIG. 14 shows the BZ oscillator as a forced-damped oscillation. (A-C) The left column shows the actual oscillations detected from the camera and the right column shows the peak intensity for all individual peaks detected when the cell stirrer was active (blue) and turned off (red).

FIG. 15 shows the average Intensity of oscillations of the cells demonstrating a forced-damped oscillator, where the figure shows oscillation peaks when cell stirrer is turned on (blue) and dampening oscillation when cell stirrer is turned off (red). The damped oscillation characteristic time scale is ca. 1.5 mins, which acts as a short-term memory for the feedback control experiments including cellular automata and computation.

FIG. 16 shows that a BZ Oscillator is a forced-damped oscillation for a longer time scale up to 30 mins. (A-C). The left column shows the actual oscillations detected from the camera per and right column shows the peak intensity for all individual peaks detected when the cell stirrer was active (blue) and turned off (red).

FIG. 17 shows the average Intensity of oscillations of the cells demonstrating a forced-damped oscillator, where the figure shows oscillation peaks when a cell stirrer is turned on (blue) and dampening oscillation when cell stirrer is turned off (red). The damped oscillation characteristic time scale is ca. 1.5 mins, which acts as a short-term memory for the feedback control experiments including cellular automata and computation.

FIG. 18 shows the chemical oscillations and peak positions of two coupled neighbouring cells, where Left (A, B) shows BZ oscillations recorded over two cells once the interfacial stirrer is active and Right (A, B) shows the corresponding peak positions vs. time.

FIG. 19 shows the phase difference between two weakly coupled cells when the interfacial stirrer was active.

FIG. 20 shows the chemical oscillations and peak positions of two coupled neighbouring cells, where Left (A-C) shows BZ oscillations recorded over three adjacent cells once the interfacial stirrers were active and Right (A-C) shows the corresponding peak positions vs. time.

FIG. 21 shows the pairwise peak differences between the three neighbouring cells. The figure shows the calculated phase differences at the peak positions with the central cell (2) as the reference.

FIG. 22 shows the discretization of continuous oscillation using CNN. Top: If a given cell detects strong oscillation (indicated by blue detected by the CNN), the state of the cell becomes “1”. The cell that was detected as weak oscillation or no oscillation at all was classified as the state “0”. Bottom: (a) Unprocessed video from the camera which was followed by (b) colour recognition from the CNN and (c) the digitisation of oscillation to state (0: light blue, 1: blue in figure C).

FIG. 23 shows the deviation from one-to-one mapping of single cell. In both (A) and (B), a cell was first stirred in high PWM (50) producing high chemical state by strong oscillation and when the same cell switches to low PWM (30), it can decay to chemical state 0 (a) or retain chemical state 1 (b), which is recognised by the CNN.

FIG. 24 shows the deviation from one-to-one mapping of single cell with nearest neighbours. (A) Shows the cell was first stirred in low PWM (30) producing low chemical state by weak oscillation, and due to the effect from the nearby high PWM (50), the chemical state switched to high. (B) The opposite case of (A) which shows the decay of the high chemical state to low due to the nearby low PWM interactions.

FIG. 25 shows the time difference between peaks in several cell as the function of peak numbers. (A-G) shows oscillations observed in seven cells of the one-dimensional BZ platform. (H) shows the phase difference between all the cells with respect to the central cell.

FIG. 26 shows the chemical clocking logic with one and two chemical states in the one-dimensional experimental platform, where (A) Chemical oscillations of all the seven cells in one-dimensional setup testing basic clocking logic without chemical states and corresponding CNN states and (B) Individual cell clocks and the global clock operations from CNN states as shown in (A). (C) Chemical oscillations of all the seven cells in one-dimensional setup demonstrating clocking logic on elementary cellular automaton and corresponding CNN states. (D) Individual cell clocks and the global clock operations from CNN states as shown in (C).

FIG. 27 shows the chemical oscillations and the state detected by the CNN of ECA rule 30, where the left figure shows chemical oscillations emerging in the one-dimensional experimental platform, with three discrete CNN states (shown in red, light blue and dark blue) in the background for all seven cells in the one-dimensional platform. The right figure shows the detailed zoomed-in image of the chemical oscillations of the second cell, where lower peaks correspond to chemical state CS₀ and higher peak corresponds to CS₁ state.

FIG. 28 shows the implementation of three elementary cellular automata, where (A) Rule 110, (B) Rule 30 and (C) Rule 250 were implemented on the one-dimensional experimental platform. The figure shows two different cellular automata states where light blue is equivalent to chemical state CS₀ and dark blue corresponds to chemical state CS₁.

FIG. 29 shows the implementation cellular automata rule 30 together with modified form, where (A) Rule 30 with symmetric interactions on both sides of each cell. (B) Rule 30 asymmetric interactions on both sides which creates a novel cellular automata rule.

FIG. 30 shows the global input and chemical states in the computational platform. (A) Global inputs states vs. grid size with k possible states on each cell stirrer and two different states on each interfacial stirrer (B) Global chemical states vs. grid size with k possible detectable chemical states on each oscillating cell.

FIG. 31 shows a demonstration of propagation event in simulation over a 5×5 grid. Left shows the initial state of the Chemit described by PWM levels. The middle shows input chemical state which could lead to propagation event (High chemical state at one of the nearest neighbours). Right shows updated PWM state using the 2D-CCA state machine showing the propagation of Chemit.

FIG. 32 shows a demonstration of replication event in simulation over a 5×5 grid. Left shows the initial state of the Chemit described by PWM levels. The middle shows input chemical state which could lead to replication event (High chemical state at one of the next nearest neighbours). Right shows updated PWM state using the 2D-CCA state machine showing replication of Chemit.

FIG. 33 shows a demonstration of competition events in simulation over a 5×5 grid. The left column shows the initial state of the Chemit described by PWM levels. The middle shows input chemical states with different possibilities of competition events which are same for all the three cases. Right shows updated PWM states using the 2D-CCA showing survival of both, none of the two and single species.

FIG. 34 shows a demonstration of random selection during multiple events in simulation over the 5×5 grid. The left column shows the initial state of the Chemit described by PWM levels. The middle shows input chemical states with different possibilities of competition events (1) two high chemical state nearest neighbours, (2) two high state next-nearest neighbours, and (3) presence of nearest and next-nearest neighbours simultaneously. Right shows updated PWM states using the 2D-CCA state machine showing propagation or replication of Chemits depending on the random selection between the various high chemical states.

FIG. 35 shows a single simulation time step over a 50×50 array. The left figure shows the initial state of the Chemits described by PWM levels with all four PWM values (Red pixels shows the position of the Chemit core). The middle figure shows updated chemical states using phenomenological state machine using initial PWM states as inputs. Right shows updated PWM states using 2D-CCA state machine completing a full single step of the simulation.

FIG. 36 shows the average propagation and replication kinetics with a variable number of random events, where (A) shows overall propagation events, (B) cumulative replication events, (C) net cumulative events which are equivalent to the difference between cumulative replication and annihilation events at each time step, (D) cumulative annihilation (death) events, in four different number of random active cell cases 10, 100, 1000 and 2500 on 100×100 cell grid

FIG. 37 shows overall propagation and replication kinetics on a different number of cell grids. (A) shows overall propagation events, (B) cumulative replication events, (C) net cumulative events, (D) cumulative annihilation (death) events, in four different number of cell grids of 10×10, 20×20, 50×50, 100×100, 150×150.

FIG. 38 shows a hybrid electronic-chemical computational state machine where (A-C) shows the concept of the proposed hybrid state machine showing information flow in chemical and electronic domains. The chemical domain comprises of two state machines and which computes in a probabilistic manner (shown in dotted) and the digital state machine computes in the digital domain in the deterministic manner (shown in continuous). The information transfer domain T (CNN) converts analogue chemical state (CS^{t}ᵢ) into the digital equivalent of a chemical state (CS^{t}ᵢ).

FIG. 39 shows a hybrid electronic-chemical “display screen” state machine showing one-to-one mapping where (A, B) shows the concept of the proposed hybrid state machine in “display screen” mode where information loops between chemical and digital domains in the absence of hysteresis and probabilistic effects. Due to a complete deterministic information loop with one-to-one mapping, the information flow is equivalent to processing in a purely digital domain as shown in B.

FIG. 40 shows a pictorial representation of interacting chemits with probabilistic outcomes which include propagation, replication and competition events together, where (A) shows probabilistic outcomes of a single Chemit where all four combinatorial outcomes are possible as a combination of propagation, replication and competition events where P1, P2 are pure propagation and P3, P4 uses a combination of multiple events as shown in (C). (B) shows probabilistic outcomes from competition events. (D) shows the presence of a simple outcome when no Chemits are present.

FIG. 41 shows a pictorial representation describing probabilistic logic using chemical cellular automata, where (A) shows two static Chemits replicating and the emerging Chemits propagates and creates different outcomes based on their interactions (see FIG. 40 B). (B) shows two Chemits replicating at different rates such that the emerging Chemits interacts differently (see FIG. 40 A). (C) shows multiple static Chemits interacting leading to different outcomes due to random selection operator in case of multiple events. (D-F) Diagrams representing a probabilistic circuit that could be mapped with equivalent Chemit Network. Each point in the Chemit network internally consists of hybrid electronic-chemical state machines.

FIG. 42 shows a pictorial representation of various events based on dynamics and interactions of Chemits, where (A) shows a Chemit random walk to due equal probability along all four sides (same PWM levels on all four sides of the Chemit core). (B) shows Chemits having directional motion with a higher probability (light red) of motion along the defined direction (higher PWM level on one neighbour as compared to other neighbours). (C) shows Competition events with possible probabilistic outcomes when two Chemit cores reach their nearest neighbours. The light blue cells are only shown to define the boundary of Chemits which do not survive, they do not represent any PWM state; and (D) shows replication and propagation events which a higher probability of replication in a specific direction (light red interaction cells leads to a higher probability of replication in the specific direction).

### DETAILED DESCRIPTION OF THE INVENTION

The present invention provides a hybrid chemical computer for performing computational operations. The computational architecture processes information in an electronically programmable chemical medium.

The use of the hybrid chemical computer can be seen as analogous to quantum computing methods, where Qubits perform a calculation, and a digital electronic system is used to both initiate and read out the results of the quantum computation. The hybrid chemical computer architecture can implement elementary cellular automaton rules, as well as having the capacity to compute the emergent behaviour of Chemits resulting from the interaction between electronic and chemical logic.

The computer of the invention is not limited to operating under two-state logic or nearest-neighbouring interactions (local couplings). The computer can be adapted to allow for fully connected couplings without creating multiple instantiations (auxiliary cells) to improve efficiency by designing equal path lengths between neighbouring and nearest neighbouring cells towards encoding universal computation. This closed-loop electronic-chemical information processing can be mapped to various chemical-electronic architectures which can be massively scaled using known CMOS electronics.

A distinguishing feature of the present case is the presence of addressable fluid channels interconnecting addressable reaction spaces within a matrix. The reaction spaces hold a reaction mixture, and the step of addressing a reaction space can initiate a chemical reaction in that space and may also sustain that chemical reaction. The step of addressing a fluid channel can provide an interaction between neighbouring reaction spaces, for example by providing localised mass transfer and mixing at the interface between the spaces.

The chemical computer previously described by one of the present inventors in WO 2020/058516 has a matrix comprising a plurality of interconnected reaction spaces holding a reaction mixture and an input device is provided to independently address each of a plurality of reaction spaces within the matrix. However, this chemical computer is only able to address the reaction spaces, and it does not address the interconnections between the reaction spaces. These interconnections are the fluid channels or gates between the reaction spaces. As a consequence, the chemical computer does not provide the level of control available in the computer of the present case.

As previously noted, having control of the interface between reaction spaces allows localized mass transfer and mixing, and therefore control of the interactions between the reactions in the neighbouring reaction spaces. In particular, controlling the interface regional controls the strength of the interactions, and also its extent across the matrix.

The first chemical computer system is a type of memory, in which the logic, or mapping, is not clear, and may be regarded as analogue. The hybrid chemical computer of the present invention uses addressable gates between the matrix spaces and these addressable gates are transformative to the operation of the computer. This the new hybrid computer has a proper digital mapping.

The hybrid chemical computer of the present allows the fluid channels or gates of WO 2020/058516 to be addressed. In the worked embodiments of the present case this is achieved by placing stirrer bars in the fluid channels, as described in further detail below.

### Chemical Computer

The chemical computer of the invention comprises a matrix, as described herein, which is provided in combination with an input device and an analytical device. Each of these components is described in further detail below, together with the operation of the chemical computer in computational operations. The computer is programmable, as it is capable of accepting information, and is capable of storing that information in the form of a modified chemical reactivity.

The chemical computer may further comprise a control unit for controlling the input device, for controlling the analytical device and for interpreting the analytical data recorded by the analytical device.

The present invention also provides a plurality of chemical computers, where the chemical computers are provided in series or in parallel.

The chemical computer may be referred to as a hybrid chemical computer for its ability to allow a computational problem to be distributed between the chemical and digital substrate.

### Matrix

The matrix is an array of interlinked reaction spaces. Each reaction space may be referred to as an element, cell or an entry within the matrix.

At its simplest, the matrix may be a sequence, such as a linear sequence, of elements. Thus, the matrix may be regarded as substantially one dimensional. However, it is preferable that the elements are arranged in a grid. Thus, the matrix may be regarded as two dimensional. In other embodiments, the elements may extend across three-dimensions.

The grid design may be adapted for the type of computation activity under consideration. For example, in the operation of the matrix for a logic gate, the spacing between reaction spaces is important for the propagation of reactivity through the matrix.

The matrix contains a reaction mixture, which mixture is distributed between reaction spaces of the matrix. The reaction mixture may be a continuous reaction mixture throughout the matrix. Thus, the reaction spaces in the matrix do not substantially isolate portions of the reaction mixture. Accordingly, the matrix is provided with fluid channels (or passages or gates) between reaction spaces to allow for the continuous distribution of the reaction mixture through the array. In the present invention, the input device may be used to address both the reaction spaces as well as the channels between those reaction spaces. Here, the positive or negative coupling between neighbouring reaction spaces, with control from the coupling gates, allows for the matrix to act as a component of a real computer.

The reaction mixture may also be a series of contacting reaction mixtures, such as contacting droplets.

Chemical reactivity initiated in one reaction space may extend as a reaction front (or wave) into neighbouring reaction spaces within the matrix. The reaction front may also be referred to as an excitation wave.

The dimensions and shape of each reaction space are not particularly limited. In practice the volume of each reaction space is minimised, where possible, to minimise the size of the matrix itself, and therefore to minimise the overall size of the chemical computer.

The shape of a reaction space is also chosen to allow for suitable packing of neighbouring reaction spaces around it, and therefore to allow for reactive communication between neighbouring reaction spaces. The reaction spaces are generally uniform in shape and volume, and with similar internal surfaces. Indeed, the reaction spaces may be identical, save for the number of interconnecting fluid passages that open into each reaction space. The number of these interconnecting fluid passages may differ between reaction spaces and this number is typically dictated by the number of neighbours to the reaction space.

The effective memory, and the durability of the memory, can be controlled by the amount of active reagents and the excitation route. In simple terms, the larger the volume of chemistry, the longer the memories can be stored.

The programmability of the chemical computer is linked to the unique number of reaction spaces in the matrix, which therefore also dictates the number of inputs that be made into the matrix. In addition to this, programmability is generated from the number of different chemical states that are accessible in response to the inputs, and the combinations of inputs.

A reaction space is in reactive communication with its neighbours within the matrix. Thus, a reaction established in one reaction space is permitted to propagate from that reaction space into neighbouring reaction spaces.

The reactive communication may be a fluid communication—typically liquid communication—between neighbours. Thus, individual reaction spaces may be connected via fluid passages. On a practical level, the reaction spaces are provided by reaction chambers, whose walls are shared with other reaction chambers. Walls between neighbouring reaction chambers, therefore between neighbouring reaction spaces, have openings to permit fluid communication between neighbours. These openings may be referred to as fluid channels, and this region of the matrix is addressable. In the preferred embodiments of the invention, a stirrer may be provided at this region, partially occupying the fluid channel.

A reaction space may be interconnected with two or more, such as three or more, such as four or more, neighbouring reaction spaces. A fluid channel generally only connects one reaction space to one other neighbouring reaction space.

The fluid passages between neighbouring reaction spaces are sufficient to allow a reaction wave to be transmitted from one reaction space to its neighbour, via a fluid channel. The transfer of material between neighbouring reaction spaces may be controlled by addressing the interconnecting fluid channels.

The fluid passages are generally uniform in shape and volume, and with similar internal surfaces. A fluid passage is typically smaller in volume than a reaction space to which it is connected. Thus, the passage serves as a passage and is distinct from a reaction space where a chemical reaction may be initiated and controlled.

One or more, and most preferably two or more, of the reaction spaces is addressable by an externally applied force. The applied force is an input for the system that is used to establish a reaction wave within the reaction space, which reaction wave is permitted to propagate into neighbouring reaction spaces.

Preferably each of the reaction spaces is individually and independently addressable. Preferably each of the interconnected fluid channels is individually and independently addressable.

The matrix is adapted for use with an input device, for applying a force into the reaction spaces and into the fluid channels.

The matrix is also adapted for use with an analytical device, for analysing the contents of each of the reaction spaces. Optionally, the analytical device may be suitable for analysing the contents of each of the fluid channels.

The matrix may be a unitary piece, for example as might be formed by injection moulding or 3D printing.

A matrix for use according to the present case may comprise 5 or more, such as 7 or more, such as 9 or more, such as 16 or more, such as 25 or more, such as 36 or more, or such as 100 or more reaction spaces.

The matrix may have an arrangement of reaction spaces within a substantially rectangular grid, such as a sustainably square grid. Here, the reaction spaces may have a substantially rectangular plan section, such as square plan section, permitting arrangement of the reaction spaces into columns and rows within the matrix.

### Input Device

The chemical computer is provided with an input device for applying a force to one or more reaction spaces within the matrix. Essentially the input device is capable of independently perturbing each reaction space in the matrix.

The input device is also for applying a force, and specifically independently applying a force, to one or more fluid channels within the matrix. The input device is capable of independently altering the mass transfer between reaction spaces and for altering the mixing at the interface between reaction spaces.

Mass transfer due to the hydrodynamic coupling between the neighbouring cells leads to interactions between the chemical reactions in the reaction spaces. For example, where chemical oscillation reactions are used within the reaction spaces, these oscillation reactions can be coupled, and the coupling can be controlled: thus, both the strength of the couplings and their extent across the matrix can be varied with alterations to the inputs to the fluid channels.

Accordingly, the matrix may be regarded as addressable by the input device.

The input device is suitable for providing an input selected from the group consisting of a mechanical force, an optical input, an electrical input, a sonic input, a magnetic input and a thermal input. Typically, the input device is for independently providing a mechanical force to each of a plurality of reaction spaces within the matrix, and for independently providing a mechanical force to one or more, such as each of a plurality of, interconnected fluid channels within the matrix.

The input device may be capable of binary operation. Here, the input device may provide a force, or provide no force at all. The input device may also be capable of providing degrees of force between a maximum force and a minimum force, or no force.

The input device is suitable for providing an input continuously, or for periods of time as necessary, and also intermittently as desired.

In the worked examples of the present case, a reaction mixture in a space is addressed by mechanical force. Thus, a magnetic stirrer bar is provided within each reaction space to provide mechanical agitation of the reaction mixture. The stirrer bar is a component of the input device, which device is also provided with a plurality of magnetic stirrers to independently operate each stirrer bar.

The magnetic stirrers are also used to demonstrate the use of the input device to provide degrees of force to a reaction mixture within a reaction space. As well as being capable of binary operation (on or off), the stirrer speed may be altered to provide degrees of force, and the worked examples shows how the changes that occur across a matrix in response to different magnitudes of force can be exploited.

The role of the a stirrer within a reaction space is to initiate and then maintain a chemical reaction, such as a chemical oscillation. A stirrer may control the reaction, such as an oscillation amplitude, by variation in the stirring speed. Reaction space stirrers also create a vortex flow within spaces cell constrained by the boundary conditions of the walls of the matrix.

The role of a stirrer within a fluid channel is to permit interaction between neighbouring reaction spaces within the matrix, by allowing localized mass transfer and mixing at the interface.

### Analytical Device

The chemical computer is provided with an analytical device for analysing the reaction spaces of the matrix.

The analytical device is provided with one or more sensors which are located about the matrix to permit the sensor to analyse the contents of one or more reaction spaces. A sensor may be provided for each reaction space, or alternatively a single sensor may be permitted to analyse a plurality of reaction spaces, such as all of the reaction spaces within the array.

The sensor is selected based on the reaction for performance, and particularly the changes in the reaction mixture during a reaction.

The analytical device may comprise an optical sensor, an electrochemical sensor, an acoustic, or combinations thereof. Typically, the sensor should be capable of 2D or 3D spatial mapping, and the time resolution for the sensor is preferably at a nanosecond level.

In the worked examples of the present case, the performance of the reaction is associated with a colour change, and an optical sensor is provided to allow for the generation of a colour map across the array.

The analytical device may be provided with alternative sensors for measurement of other characteristics of the reaction mixture.

In one embodiment the analytical device may be provided with a range of sensors for detecting a plurality of characteristics of the reaction mixture.

The analytical device may comprise an optical sensor.

The data collected by the analytical device may be communicated to a control unit for analysis.

The analytical device may be controlled by the control unit. The control unit may coordinate the measurement of analytical information with the input.

The analytical device may be permitted to operate continuously to record analytical data from the reaction spaces.

### Reaction Mixture

The matrix is provided with a reaction mixture, which is distributed across a plurality of reaction spaces.

The reaction mixture typically comprises one or more reagents, optionally together with a solvent and optionally together with a catalyst. The reaction mixture may be a liquid mixture, such as a solution.

The reaction of the reaction mixture is associated with a change in a detectable characteristic of the reaction mixture, such as an optical characteristic of the reaction mixture. The change in a characteristic of the reaction mixture is detectable by the sensor of the analytical device.

Typically, the reaction mixture is for a non-equilibrium reaction, for example a reaction that is a non-equilibrium thermodynamic reaction.

The reaction mixture may be for a reaction that allows a large number of chemical states to be accessed, but all these states can be interconverted as a result of external operations.

The reaction mixture may be a reaction mixture for a chemical oscillator reaction, a polymerisation reaction, a molecular synthesis, or an autocatalytic process.

The reaction mixture may be a reaction mixture for a chemical oscillator reaction. Such reactions are examples of non-equilibrium thermodynamic reactions.

When the reaction mixture is capable of periodic changes in the concentration of one or more species within the mixture, additional encoding can be achieved. Such an oscillator reaction may be initiated or altered by application of an input to the reaction mixture, applied by the input unit. In the present case, the oscillation is associated with a change in a characteristic of the reaction mixture that is detectable by a sensor of the analytical unit. The oscillation may be associated with a change in colour.

In the present case, the reaction may be one where there are three or more observable colours in the reaction

Where the reaction mixture is a chemical oscillator reaction, a localised reaction mixture within a reaction space may be regarded as an oscillation clock. The methods of the invention may therefore look at changes in the oscillation frequencies, which are influenced by the applied force, where such a force is applied to a reaction mixture in a reaction space.

In the absence of an applied input, a chemical oscillation localised in a reaction space may be essentially random, or chaotic, or not activated at all.

However, when a reaction space is addressed, local order within that reaction space may result. That local order may permeate, or propagate, from that reaction space into neighbouring reaction spaces. Analysis of the reaction spaces may show changes in the characteristics of the reaction that are associated with the propagation of the reaction from one reaction space into another. This may be referred to as a reaction wave which is established by the applied force from the input device.

Where a plurality of reaction spaces is addressed, each of these reaction spaces may set up a reaction wave that propagates beyond each addressed reaction space. A reaction mixture within a reaction space may receive a reaction wave from a neighbouring reaction space, and that reaction space may receive a plurality of reaction waves from a plurality of neighbouring reaction space, for example providing a constructive interference within the reaction space.

The chemical oscillator reaction may be selected from the group consisting of a Belousov-Zhabotinsky (BZ) reaction, a Briggs-Rauscher reaction and a Bray-Liebhafsky reaction, such as a Belousov-Zhabotinsky (BZ) reaction.

Other autocatalytic reactions can also be used, where the chemical change under consideration is reversible.

A BZ reaction may use bromine, for example as bromate.

In the present case, the reaction mixture may use an iron catalyst within the Belousov-Zhabotinsky (BZ) reaction mixture, such as iron catalyst oscillating between Fe(II) and Fe(III). The iron catalyst may be ferroin, [Fe(bpy)₃]^{2/3+}.

A chemical oscillation reaction may be a reaction which is chaotic in the absence of an applied force.

A chemical oscillation reaction may be a reaction where the oscillations are suppressed in the absence of an applied force.

A reaction in a reaction space may be initiated and optionally also sustained by the application of a force into the reaction space.

The worked examples in the present case demonstrate show the use of an applied mechanical force to a reaction mixture to initiate and sustain a BZ reaction.

The inventors have found that a reaction mixture within a reaction space that is addressed by the input device gives rise to a reaction wave that extends from the reaction space into neighbouring reaction spaces. Thus, the change in the reactivity within a certain reaction space is capable of altering the reactivity of the reaction mixture in another reaction space.

The reaction wave that is propagated from a reaction space typically has a limited extent by which it can alter reactivity in neighbouring reaction spaces. The conditions under which a force is applied to a reaction space may be selected to ensure that there is a limited extent of influence.

The present inventors have found that the propagation of a reaction into neighbouring reaction spaces may be used as the basis for the operation of the chemical computer as a logic gate, for example as an OR gate. Similarly, the limited extent of the propagation may also be exploited for operation of the chemical computer as a logic gate, for example as an AND gate.

The inventors have also found that some reactions, such as oscillating chemical reactions, retain a memory of their earlier perturbation, which refers to the earlier addressing of the reaction spaces by the input device. Thus, a reaction pattern may persist in the matrix for a period of time after an initial input into the matrix. In some embodiments, the memory may be permitted to degrade before further inputs are made into the system. In other embodiments, further inputs are made into the system whist there is a persistence of an earlier reaction within the matrix.

### Control Unit

The chemical computer may further comprise a control unit, which may itself be a computer or a plurality of interlinked computers.

The control unit is suitably programmed to control the input device, for example the control unit is capable of controlling which reaction spaces within the matrix are addressed, and the duration, and optionally also the degree, of the forces applied in the address.

The control unit is suitably programmed to control the analytical unit, for example the control unit is capable of instructing the analytical unit which reaction spaces are to be analysed, and for how long. The control unit may also receive analytical data from the analytical unit, and it may analyse the analytical data.

A control unit may be used to control a plurality of matrices together with associated input units and analytical units.

The control until may be the interface through which a user is capable of operating the chemical computer.

### Methods and Uses

The present invention provides the use of a chemical computer as a computer. The operation of the chemical computer is as described herein. Thus, signal processing and computing may be achieved by appropriate addressing of reaction spaces and appropriate addressing of fluid channels, with the analytical system used to acquire reaction information from reaction spaces, such as across the matrix.

The worked examples in the present case describe in detail the operation of a chemical computer, including steps for clocking the matrix to achieve internal synchronicity, and methods for chemical cellular automata and state space expansion.

Thus, the invention also provides a computer comprising one or more chemical computers of the invention, which may be arranged in series or parallel.

The present invention provides the use of a chemical computer as a logic gate. The operation of the chemical computer is as described above.

The chemical computer has programmability resulting from the discrete use of individual inputs to reaction spaces within the matrix. These inputs give rise to reaction waves within the matrix that combine to generate a unique reaction result that is the reactive response to those inputs.

The inventors have found that the reaction result in the matrix is repeatable for the same series of inputs. Thus, the system is reliable and reproducible. The system can therefore recognise a particular output that is linked to a particular input.

The present invention also provides the use of the chemical computer in a synchronised fashion, where controlled inputs to the system, for example by addressing the fluid channels, may synchronise the matrix. As described herein, the inventors have found that addressing the fluid channels allows coupling between nearest-neighbour cells, which can bring these cells into synchronicity. Here, the addressing of the reaction spaces may also be controlled to minimise oscillation drift. Thus, the inventors have found that addressing the reaction spaces by pulsing the reaction, for example with stirrers, and allowing rests between pulses minimise phase shifts in the oscillation signals.

Thus, the present invention provides a method of synchronising a chemical computer, the method comprising addressing a fluid channel between two neighbouring reaction spaces in a matrix, where chemical oscillation reactions are provided in each of the two reaction spaces, thereby to alter the phase of a reaction in one reaction space.

Preferably, the method comprises addressing a plurality of fluid channels, with each fluid channel between two neighbouring reaction spaces in a matrix, where chemical oscillation reactions are provided in each of the reaction spaces, thereby to alter the phase of a reaction in a plurality of reaction spaces.

The method may provide synchronicity in the oscillation reactions in the matrix.

Generally, the methods of the invention provide for the use of the chemical computer as a computer. The chemical computer allows for inputs to be made into the matrix, by addressing reactions spaces, and the development of chemical information across the matrix may be analysed and may be influenced by the control of the interface between neighbouring reaction spaces, which controls material transfer between those spaces.

The computer of the invention allows for the propagation of chemical information across the matrix, as well as replication of chemical information across the matrix.

In one embodiment, the invention provides for the use of the chemical computer to propagate, or transfer, a chemical signal, such as an oscillation, as determined by analytical analysis, for example of colour change, from one reaction space to a neighbouring reaction space. Such transfer may be achieved by addressing a fluid channel. Here a source reaction space is provided with a chemical oscillation reaction.

In one embodiment, the invention provides for the use of the chemical computer to replicate, or copy, a chemical signal, such as an oscillation, as determined by analytical analysis, for example of colour change, from one reaction space to a neighbouring reaction space. Such replication may be achieved by addressing a fluid channel that connects the neighbouring reaction spaces. Here a source reaction space is provided with a chemical oscillation reaction.

### OTHER OPTIONS

Each and every compatible combination of the embodiments described above is explicitly disclosed herein, as if each and every combination was individually and explicitly recited.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described. Where technically appropriate embodiments may be combined and thus the disclosure extends to all permutations and combinations of the embodiments provided herein.

Certain aspects and embodiments of the invention will now be illustrated by way of example and with reference to the figures described above.

### EXAMPLES

The following examples are provided solely to illustrate the present invention and are not intended to limit the scope of the invention, as described herein.

### General Experimental

All reagents were used as received. Ferrous sulphate heptahydrate was sourced from Sigma Aldrich, ≥98%. 1,10-phenanthroline was sourced from Sigma Aldrich, ≥99%. Sulphuric acid was sourced from Fisher Scientific, ≥95% analytical reagent grade. Malonic acid was sourced from Sigma Aldrich, Reagent Plus 99%. Potassium bromate was sourced from VWR and Sigma Aldrich >99%. Deionised (D.I.) water in the preparation of the stock solutions and experiment was sourced from PURELAB® Option-S/R 7/15. 0.001 M of the ferroin solution was prepared by first dissolving 2.78 g of ferrous sulphate heptahydrate and 5.40 g of 1,10-phenanthroline in 10 mL of D.I. water. The resulting solution 0.1 M ferroin indicator was further diluted to 1 L with D.I. water. 1.0 M of sulphuric acid solution was prepared by diluting 56 ml of concentrated (≥95%) sulphuric in D.I. water and made up to 1 L. 1.0 M of malonic acid was prepared by dissolving 104 g of malonic acid in 1 L of D.I. water. 0.5 M of potassium bromate solution was prepared by dissolving 83.5 g of potassium bromate in 1 L of 1 M sulphuric acid.

### Platform Hardware

The platform architecture was designed using computer-aided software (OnShape) and printed with Stratasys Connex500 3D printer using the VeroWhitePlus material that holds the chemical mixtures and FullCure 720 material for the rest of the platforms. The overall platform was placed in a box made out of 5 mm translucent acrylic sheets (supplied by Wanna Ltd.) and support structure using v-slot linear rails (supplied by Ooznest Ltd.) to ensure a more even distribution of light for image acquisition. The DC motors were equipped with Neodymium-based permanent magnets to ensure each stirrer bars can be addressed individually. The addressable DC motors were controlled by Arduino Uno REV3 SMD with Adafruit 16-Channel 12-bit PWM/Servo Shield—I2C interface. The camera used was Logitech C 920 HD PRO WEBCAM. The platform runs fully on Python language and the code used for the platform is available in the GitHub link stated in data and code availability.

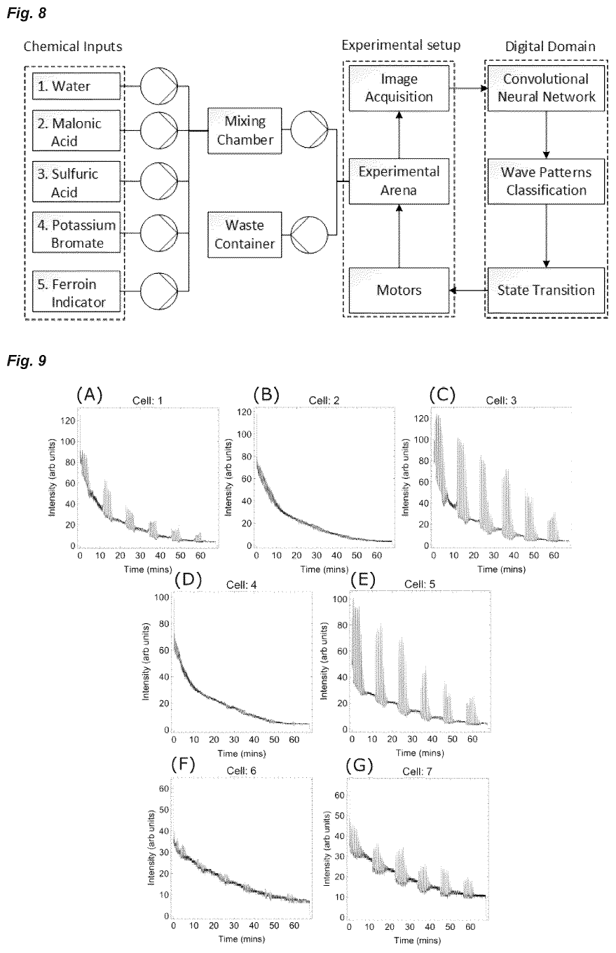

The overall hybrid electronic-chemical computational platform consists of three main control domains as shown in FIG. 8, namely the (1) chemical domain which consists of stock solutions required for BZ reaction that were pumped using syringe pumps sequentially in the right proportion into the mixing chamber. The mixing chamber contains a magnetic stirrer bar that rotates at 140 rpm constantly to ensure the stock solutions were well mixed. Using another pair of syringe pumps, the reaction mixture in the mixing chamber was then transferred to the 3D printed experimental arena with stirrers. In this experimental setup, the rotation of stirrers is controlled by DC motors equipped with Neodymium-based permanent magnets located at the bottom of the arena. Each motor speed and direction can be individually addressed by the supported electronics control. The BZ chemical oscillations occurring in the experimental arena on the response of stirrer actuation were then observed and recorded by a camera.

These temporal oscillatory patterns were then passed into the (3) digital domain where further information processing occurs. The oscillatory patterns were classified into three different states using a convolutional neural network (CNN). There are three different classification states, RED, LIGHT BLUE and BLUE. These classified states were used for creating a common chemical clock over all the cells as well as the basic programmable chemical states (CS) for computation.

The chemical clocking logic is used over all the experiments as a sync signal for a single feedback loop step. These patterns in the given time frame were then converted into the observed chemical states, high state: CS1 and low state CS0 by using a Finite State Machine (FSM). This FSM reads the temporal CNN states over a given time and return the digital CA state based on the observed oscillatory behaviour and resets.

The hybrid electronic-chemical computational logic is then implemented on these chemical states using various problem-dependent state machines which are discussed further in the later sections. The hybrid electronic-chemical computational logic utilizes these chemical states which then dynamically controls the stirrers speed and completes the feedback control loop over the complete experimental period. This feedback loop together with the chemical clocking logic were used to create novel one-dimensional and two-dimensional Chemical Cellular Automata (CCA) and performing useful computation by solving combinatorial optimization problems.

Once an experiment is finished, the remaining solution was drained into the waste container using a pair of syringe pumps and the experimental arena underwent a series of rinsing and cleaning cycles to get ready for the next experiment. This is the overall description of the complete experimental protocol and full details of individual steps and experimental protocols is discussed in the following sections.

### Syringe Pumps and Tubing

The solutions were pumped by 14 pumps (model C3000, Tricontinent) with 12.5 mL syringes except for ferroin solution, 5 mL. The pumps were connected using a RS232 port and using an in-house PCB that allow up to 15 pumps on a single RS232 bus. The PyCont library was used to control the pumps which can be found at https://github.com/croningp/pycont. PTFE plastic tubing with an outer diameter of ⅛ inches (3.175 mm) was cut to a specified length and connected using standard flangeless fitting and ferrule for ⅛ inches outer diameter tubing with a flat bottom (supplied by Cole Palmer Ltd.).

### Electronics

The DC motor speed (RPM) was controlled by applying a modulated voltage between 0-6 V using a Pulse Width Modulation (PWM) signals from the microcontroller prototype board Arduino UNO. To control both motor speed and direction, Adafruit Motor Shields (Ver. 2) which were stacked to control multiple DC motors for each Arduino UNO. The motors were directly connected to the Motor Shield using pin-screw terminals with a wire connection scheme to separate cell motors and interfacial motors. Each shield can control the speed and direction of four motors and uses I2C communication protocol to communicate with the microcontroller unit. Each motor shield consists of 5 address-select pins (via soldering) to provide an address for communication to the specific shield. By stacking multiple shields for the overall platform, each motor can be addressed by a combination of shield address and motor ID (1-4) on the shield.

To power all the motors, programmable 2-channel external power supply unit (RS Components Ltd.) capable of supplying enough current to power all 133 DC motors were used. To simplify the nomenclature of the motors and addressing, four Arduino UNO boards, two each for cell stirrer and interfacial stirrer motors were used. These four microcontroller units allowed us to individually address 133 motors for the two-dimensional platform. Table S2 shows the number of motors, shields and prototype boards used for one- and two-dimensional setup. Each motor address is given by the combination of three addresses: {Arduino UNO ID, Motor Shield Address, Motor Number}. The TriContinent C 3000 pump control uses daisy-chaining to connect with a physical address pin (0-15) on each pump. An in-house designed PCB for daisy-chaining pumps data and power pins were employed for the TriContinent C 3000 pumps. A 24 V power supply from RS Components were used to power the pumps.

### Control Software

The software layer that is responsible for controlling the platform can be divided into two parts, namely (i) The firmware that runs on all the Arduino UNO boards to activate all the individual motors with I2C communication and (ii) A Python script that communicates between the Arduino and Python via the serial interface as well as an in-house developed library to control TriContinent C 3000 series pumps via a python script. The firmware for Arduino UNOs was written using opensource Arduino IDE and an available library from Adafruit Industries Ltd. were used to communicate with the stacked motor shields. A high-level interface python script was written to communicate with the Arduino via serial interface (using pyserial). This ensures a more intuitive way to program the experimental system by a researcher.

Adafruit 16-Channel 12-bit PWM/Servo Shield—I2C interface is employed to generate the PWM signals that would actuate the motors. By using the corresponding Arduino library: https://github.com/adafruit/Adafruit-PWM-Servo-Driver-Library, any users can send different commands for PWM signals. The function is as followed “setPWM (pin, direction, speed)”, where pin refers to a unique motor and direction refers to the direction of the motor i.e. 0 is clock-wise and 1 is anti-clockwise and speed generate a PWM signal resulting in specific RPM.

### Autonomous Platform Workflow

Upon starting the experimental script, water, malonic acid, sulphuric acid, potassium bromate and ferroin solution was pumped into a mixing chamber sequentially. The resulting mixture was then pumped into the experimental arena. Once completed, all the stirrers were activated at 50 PWM for 1 minute followed by deactivation of all stirrers for 4 minutes. This homogenises the solution in the platform and break down of oscillation of the bulk solution from the mixing chamber. This was followed by activation of interfacial stirrers and pulsing of cell stirrers for 5 minutes. The rationale for the following step was to ensure the BZ reaction in cells are synchronous prior to setting the initial condition for a given experiment. Once the pre-treatment was done, the experiment was initialised and a dynamic feedback loop interfacing between the chemistry, CNN and DC motors occur for a specified step (using a chemical clock). Upon completion, a cleaning cycle occurs where the solution in the platform was drained and 3 times the rinsing cycle with water occurs. If the result was not satisfied in the prior experiment, the loop described above occurs again and the last step of the previous experiment will be used as the initial state of the new experiment, otherwise the platform halt.

### Chemical Oscillator

Amongst the oscillatory reactions, the Belousov-Zhabotinsky (BZ) reaction was chosen due to its robustness and easily accessible reagents. For instance, the BZ reaction can last up to an hour or more with consistent oscillations even in a closed system. This allows the system to be programmed and observed without the necessity of constant replenishment of reactants. Admittedly, continuous-stirred tank reactor (CSTR) system would allow lengthier experiment and a more controlled O2 uptake may provide a more consistent period and amplitude of oscillation. Reports have shown the oxygen inhibition effect which may change the behaviour of the system ever so slightly with the decarboxylation of malonic acid which cause CO2 bubbles. However, we opted to use a batch reactor system due to its simplicity and we have implemented chemical clocking logic which would compensate any phase-drift and change in amplitude of the oscillation that may occur in the experiment which is discussed in Section 4. An upside of the closed system with 3D compartments, as opposed to CSTR can be attributed to the fact that the state of localized chemical oscillations and hysteresis effects can be preserved and could be utilized for implementing logic and computation. This localized memory-like effect provides the key towards the implementation of closed-loop dynamic logic and computation. The iron-catalysed variant (ferroin) of the BZ reaction was used in all the experiments. Ferroin was used as the indicator and a single-electron redox catalyst, which results in colour changes from red when the iron is in (+2) state and blue when the iron is in (+3) state as shown in the equations below.

$${{Fe}^{2 +} ( phen )}_{3} ↔ {{Fe}^{3 +} ( phen )}_{3} + {e}^{-} \qquad ( 1 )$$

$$Red ( Reduced ) ↔ Blue ( Oxidised ) \qquad ( 2 )$$

The oscillation of the BZ reaction can be easily stimulated mechanically (Parrilla-Gutierrez et al.) and photochemically (Gentili et al.). Mechanical stimuli via magnetic stirring were employed in this system due to its simplicity towards large-scale implementation and its capability to create a well-controlled local coupling between the nearest neighbouring cells.

The stock solutions for the automated platforms were prepared as followed:
Ferroin: 0.1 M of the solution was prepared by dissolving 2.78 g of ferrous sulphate heptahydrate and 5.40 g of 1,10-phenanthroline in 10 mL of deionised water. The solution was then further diluted to 0.001 M for the experiment.
Sulfuric Acid: 1.0 M of the solution was prepared by diluting 56 mL of concentrated H₂SO₄ to 1 L of deionised water.
Potassium Bromate: 0.5 M of KBrO3 solution was prepared by dissolving 83.5 g of KBrO₃ in 1 L of 1 M H₂SO₄.
Malonic Acid: 1.0 M of the solution was prepared by dissolving 104 g of CH₂(COOH)₂ in 1 L of deionised water.
Deionised water: PURELAB® Option-S/R 7/15 was used as the source of all water used in the experiments and preparation of stock solutions.

### Experimental Protocols

The complete schematic diagram of the single experiment protocol is shown in Scheme 1. A single experiment takes the following steps.

The following reagents were added sequentially via Tricontinent C-Series syringe pumps into the mixing chamber during the sequential chemical inputs stage:
1) 18 ml of water;
2) 18 mL of 1 M malonic acid;
3) 12.5 mL of 1 M sulfuric acid;
4) 19 mL of 0.5 M potassium bromate in 1 M sulfuric acid;
5) 2.5 mL of 0.001 M ferroin indicator.

When the ferroin indicator was added into the reaction mixture, immediate colour change from red to blue was observed whilst the mixture was stirred. The reaction mixture was then pumped into the respective platforms; note that the volume of reagents mentioned above was used in the 1D platform and they were scaled up by four times for the 2D platform.

After the mixture was pumped into the platform, the mixtures went through a series of pre-experiment treatment using magnetic stir bars that were controlled by DC-motors as followed:
1) All the stirrers were activated at 50 PWM (max PWM: 255, 8-bit) for a minute;
2) Deactivation of all stirrers and the solution was rested for four minutes;
3) Activation of interfacial stirrers and pulsing of cell stirrers for five minutes.

Scheme 1—The workflow of a single experiment. The schematic figure shows experimental protocols carried out in every experiment which consists of the stabilisation period, a synchronised oscillation in all the cells, actual experiment, cleaning, and a pre-defined logic to run the following experiments.

### Camera and Configurations

The 1D and the 2D platforms used Logitech C920 HD PRO Webcam. The webcam was situated 20.5 cm above the 1D platform arena and 33.4 cm for the 2D platform arena. These distances were chosen carefully to fill the complete field-of-view to get the best resolution. The video was configured to 1280×720 pixels and 10 frames per second (FPS) for the 1D platform while the 2D platform was configured to 800×600 pixels and 15 FPS. The camera was configured using the opensource GUVC View software. It is important to note that the selected parameters were chosen based on the light levels in the experimental enclosure such that oscillatory coloured patterns are distinct and clear for image processing. XVID compression was used in both of the platforms. During the experiment, the camera stream was fed into a running Python (3.7.1) OpenCV (3.4.4) script.

### Control Experiments

Multiple control experiments were performed to find the optimal parameters for controlling the stirrers to initiate strong or weak oscillations and localized nearest neighbour couplings between the cells necessary for the implementation of programmable hybrid electronic-chemical logic. This also includes the selection of appropriate dimension of individual reactor cells, stirrers' positions and wall dimensions to create ideal hydrodynamic coupling between the nearest neighbouring cells. Depending on the requirements, experiments were performed in one- and two-dimensional platforms.

### Basic Oscillation Test and Recording

As an initial step to estimate the time scale and stability of chemical oscillations in the closed system, basic actuation test by constantly stirring the cell stirrers up to an hour was carried out and the oscillations over all the cells in the one-dimensional platform was recorded. Constant oscillations over all the cells with a minor decrease in measured intensity up to one hour was observed. This provides the evidence for running each experiment up to an hour without changing chemical reagents. BZ oscillations recorded over an hour on three neighbouring cells were observed to have stable oscillations with the same frequency and intensity over the three neighbouring central cells (in a one-dimensional platform having cells in total).

To prove that the effect of the cell stirrers is localized to the specific cell in the absence of the interfacial stirrers, experiments on the one-dimensional platform was carried out by actuating every alternate cell (1, 3, 5 & 7) up to 5 seconds and record the oscillations. The recorded oscillation data over all the seven cells is shown in FIG. 9, where activated cells (1, 3, 5, and 7) shows strong oscillations due to stirrer actuation as well as dampening effect when the stirrers were inactivated.

The other cells 2, 4, 6 shows almost negligible oscillations due to extremely weak interactions in the absence of hydrodynamic coupling when the interfacial stirrers were inactivated. This proves that actuation of only cell stirrers creates only local interactions and hence, can be used to program localized chemical states in a controlled manner.

Hydrodynamic Tests with Markers to Test Neighbouring Cell Coupling

To investigate the coupling between the neighbouring cells by activating interfacial stirrers, we performed hydrodynamic tests by using ink as a tracer to monitor fluid flow by tracking the colour change between the cells. The interfacial stirrer was placed symmetrically between the two cells, such that the interfacial stirrer couples the two vortices of the neighbouring cells where the cell stirrers were active. Utilizing this concept, the experimental setup was designed considering three important criteria:
1. Vortex of the fluid must be localised within a single cell when the cell stirrer is turned on and the interfacial stirrer is inactive. The vortex in the cell must be contained.
2. On activating the interfacial stirrer when the two neighbouring cell stirrers are also active, the interfacial stirrer should be able to couple the two cell vortices causing neighbours to interact with one another.
3. The fluid flow at the interface should create a symmetric bidirectional flow between the two cells.

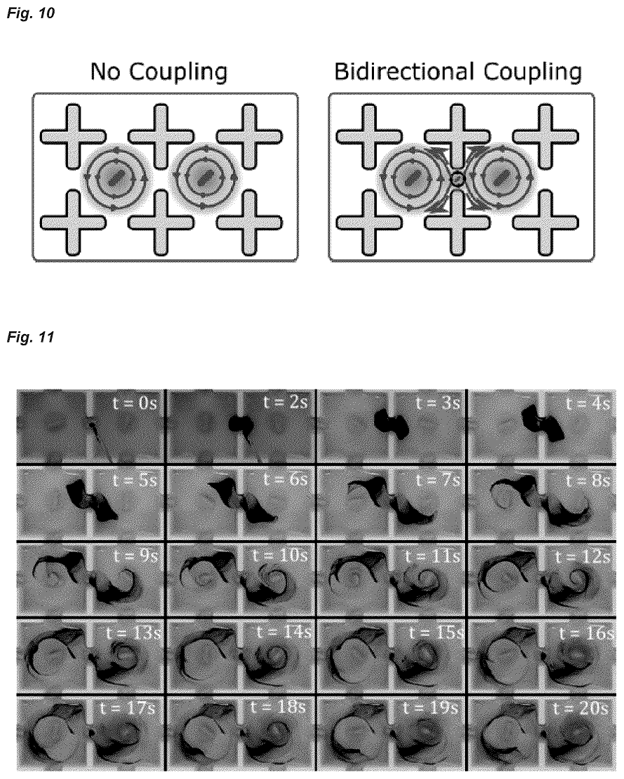

FIG. 10 shows a pictorial representation of the two neighbouring cells whose vortices do not couple with each other when interfacial stirrer is inactive and hence no coupling and symmetric bidirectional coupling occurs when interfacial stirrer is active. All the active cell stirrers rotate in the same direction and interfacial stirrers rotate in the opposite direction to the active stirrers.

In principle, it is possible to run Computational Fluid Dynamics (CFD) simulations to simulate the fluid flow over the network of cells with interacting vortices. However, CFD simulations with moving objects are usually computationally intensive. Hence, experiments using ink as a marker to monitor fluid flow were carried out. Diluted “Sumi” ink was used as a fluid marker to monitor the fluid flow.

As the first example, two neighbouring cell stirrers were activated at the same PWM level or RPM. It was then followed by the activation of the shared interfacial stirrer between the two cells. A drop of ink using a fine syringe needle was placed on the interface and the flow of the ink was recorded by a camera. Due to symmetric flow of fluid between the neighbouring cells, the ink flows symmetrically between two neighbouring cells and as soon as the ink boundary travelled close to the centre of the cell stirrer, it coupled strongly with the inner vortex in the cell and this led to homogeneous mixing in both cells, see snapshots in FIG. 11. So, by activating the interfacial stirrer, the two neighbouring cells with the same PWM values can be coupled with each other symmetrically which could be used for programming the couplings between the cells for computation experiments.

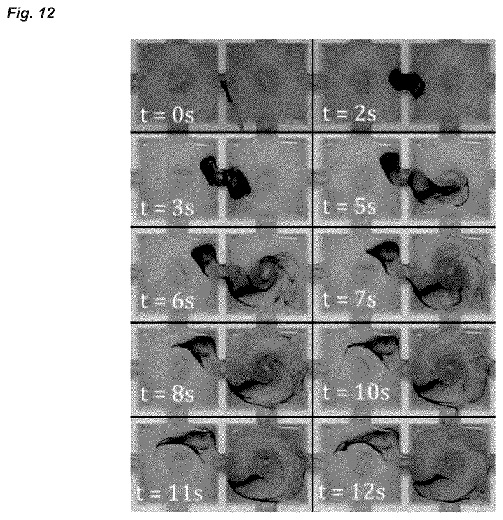

As an additional test, we performed a similar experiment with two active cell stirrers but with different PWM levels and activating the interfacial stirrer between them. Due to the difference in the speed of the cell stirrers, the cell-cell coupling becomes asymmetric as demonstrated in FIG. 12. The PWM of the right cell is stronger as compared to the left cell. At the start, the coupling appears to be symmetric, but as soon as the ink boundary reaches strong vortex of the right cell, it starts mixing well throughout the cell, which was not observed in the left cell at the same time. The asymmetric coupling between the cells is much harder to quantify at different speed levels as compared to the symmetric coupling due to non-linear coupling between the interactions fluid flow within the cells and at the interface. Thus, symmetric coupling was used as the basis for computation experiments.

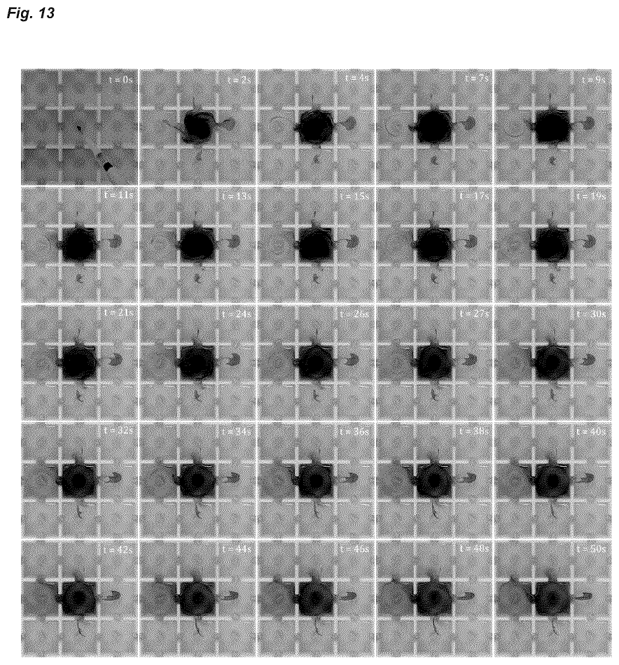

Additionally, hydrodynamic coupling tests in the two-dimensional platform were performed to demonstrate coupling between neighbouring cells occurs only when interfacial stirrers between them are active. To demonstrate this effect, a 3×3 grid of cells with only one set of cell stirrers and interfacial stirrer between them was activated. In this case, instead of dropping ink at the interface, a drop of ink was placed in the central cell and then activate the cell stirrer. Subsequently, we activate the interfacial stirrer between the central and the left cell. A series of snapshots in FIG. 13 shows mass transfer between the two active cells and as a result the concentration of the ink in the central cell decreases and increase in concentration on the left cell. No mixing was observed in the rest of the cells where interfacial stirrers were inactive, note that the apparent leakage observed on the right, top and bottom of the cell is due to the experimenter whilst injecting the ink.

These hydrodynamic tests gave a good indication about developing electronic actuation operations which could be used for creating a hybrid electronic-chemical logic. However, understanding the effect of stirring on the oscillation of BZ reaction together with the mass transfer is complex phenomena. In the later sections, further tests were carried out to investigate BZ oscillations and their coupling between cells.

### Chemical and Hydrodynamic Tests

An experimental investigation was performed to visualize the temporal pattern of the emerging fluid vortex on the stirrer actuation. Initially, the cell was filled with water with ink (Sumi) as a fluid flow marker, was carefully injected at the bottom of the well. Upon activation of the stirrer, the ink disperses quickly and creates a steady state profile along the complete cross section.

The temporal snapshots showed the homogeneous mixing of the ink in approximately 12-14 seconds. The no fluid flow zone at the edges of the cells can be seen clearly at times 12-18 seconds. This no flow zone at the edges helps to avoid the interactions between next-nearest neighbours. The interactions between the nearest neighbours occurs due to the coupling of vortices due to interfacial stirrers and the no fluid flow zone minimizes the coupling between diagonally placed cells.

Due to the presence of no flow zone at the edges, no oscillations occur at the edges which leads could lead to inhomogeneity in the measurement. However, the region of interest detected by the CNN is smaller than the dimension of the cell such that the measurement does not suffers from the spatial inhomogeneity

### Effect of Stirring Rate on Chemical Oscillations

We investigated the effect of stirring rate i.e. PWM levels on the observed amplitude of the chemical oscillations. We used one-dimensional BZ platform and scan the PWM levels in the range [20, 80] with the interval of 2 unit in forward direction and waiting for 40 seconds at each PWM value. We observed continuous increase in the amplitude of oscillations with increase the in stirring rate. At the lower PWM levels, the rate of increase in amplitude is slow and weaker oscillation persists in the PWM range 20-30. In the range 30-60, the oscillation amplitude increases at higher rate which eventually saturates in the range 60-80. This transition in amplitudes from the lower to higher PWM levels allows us to select from a range PWM levels to define programmable states based on temporal oscillation patterns as discussed in detail in the next section.

### Phenomenological Behaviour of the Hybrid Electronic-Chemical System

Besides the optimization of the cell dimensions and enhancing control of the cell-to-cell coupling, the oscillator chemistry plays a significant role in the observed spatiotemporal behaviour of the overall experimental platform. In this section, we will investigate the dynamics of chemical oscillations within a single cell as well as coupling dynamics between the nearest neighbouring cells due to the actuation of the cell and the interfacial stirrers. The different behaviours observed were later used to define hybrid electronic-chemical logic for dynamic closed-loop experiments for implementing cellular automata and computation. The various phenomenological behaviours as described are as followed:
1. Single cell
a. Chemical oscillations only appear when the cell stirrer is active which is equivalent to a forced oscillator.
b. Once the active cell stirrer is deactivated, the forced oscillator turns into a damped oscillator and after a few cycles, the chemical oscillations disappear.
2. Coupled neighbouring cells
a. Neighbouring cells do not interact with each other when the interfacial stirrer is inactive.
b. On activating interfacial stirrer, weakly coupled neighbouring cells come in phase independent of their initial phase differences.
c. Interactions between two or more cells in one and two-dimensional geometry are confined to nearest neighbours only.

### Chemical Oscillator as a Forced and Damped Oscillator

BZ chemical reaction oscillating under the actuation of a cell stirrer and falling back to the ground state once the stirring actuation is turned off can be described as a combination of a forced and a damped oscillator. As soon as the cell stirrer is active, the system reaches an active oscillating state in around 1-2 oscillations. However, it takes at least three oscillations to come back to the ground state once the cell stirrer is deactivated. This damped oscillatory behaviour can be used as short-term localized chemical memory for developing key components of the hybrid electronic-chemical logic such as clocking signal, self-interactions etc. To estimate the time scale of the dampening effect, we performed experiments on the one-dimensional experimental setup by activating the cells for a finite amount of time and followed by deactivating them. The oscillations over the complete setup were recorded over the whole experimental time.

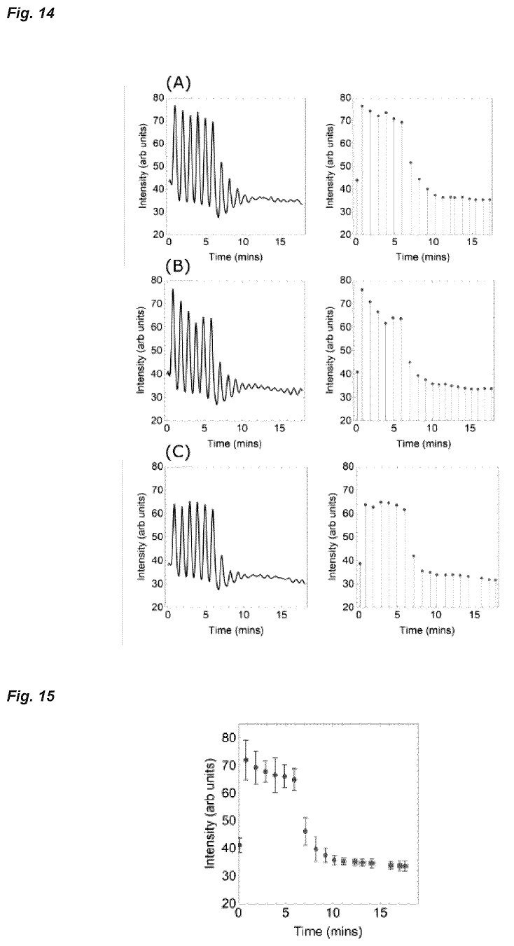

FIG. 14 shows the oscillations of three different cells and the position of the peaks with time. In all three cells, it only took time scale of one oscillation ca. 30-40 seconds to reach the forced oscillation state from the ground state, and around 3-5 oscillations to arrive ground state once the cell stirrer was deactivated.

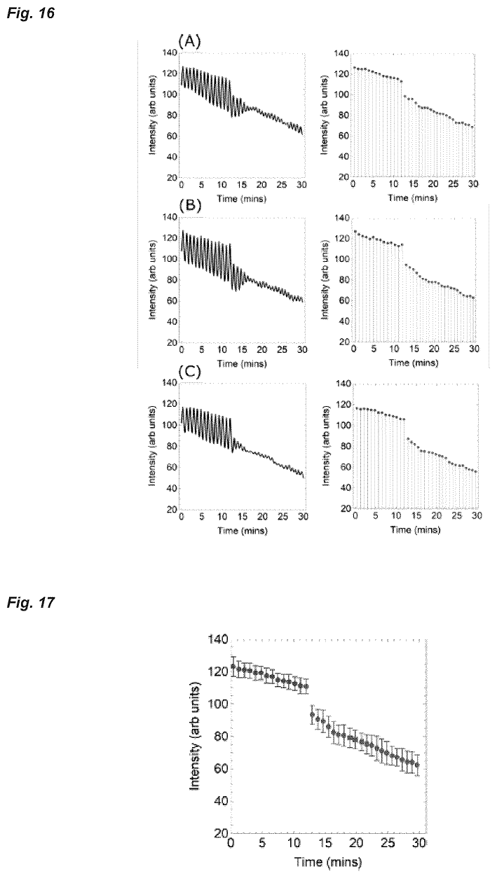

From the experimental data presented in FIG. 14, the average oscillation intensity and the time of the peak position together with the standard deviations as shown in FIG. 15. The position of the peak of all the oscillations seems well-defined and the characteristic timescale for damping can be estimated. Additional experiment for a longer time scale and another set of results for three cells in one-dimensional setup is shown in FIG. 16 and FIG. 17.

### Interactions Between Nearest Neighbour Cell Oscillations

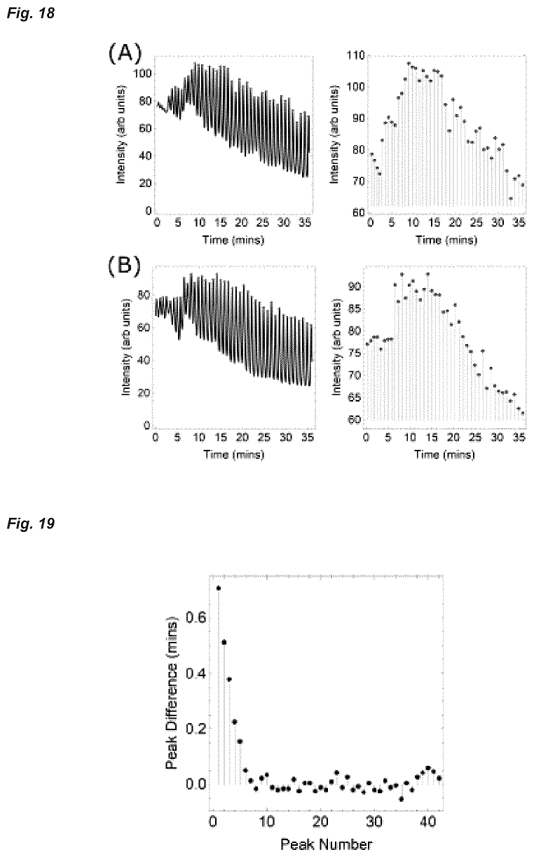

A key feature necessary for the experimental platform towards efficient computation is to develop a “decision-making” logic based on the observed chemical oscillations. As the chemical oscillations within different cells could emerge at different times, it is important to create a clocking logic which acts as a sync signal, analogous to the one used in electronic devices, to update the temporal oscillatory states occurring in all the cells. This decision-making logic could be massively simplified if all the oscillations occurring in the cells stays in the same phase. This could be possible by creating a weak interaction amongst all the cells by utilizing combinations of cell and interfacial stirrers. To investigate the effect of interfacial stirrers between the two oscillating nearest neighbouring cells with cell stirrers activated, we monitored the phase difference between the oscillations. We started by actuating two cells stirrers at different initial times so that we can observe an initial phase difference between the emerging chemical oscillations. Then, the interfacial stirrer was activated between them followed by monitoring the phase difference between the two neighbouring cells. FIG. 18 shows the observed BZ oscillations and their peak positions recorded over the time scale of 35 mins. When the interfacial stirrer was turned on, minor change in the intensity of the BZ oscillations was observed. This is due to stronger hydrodynamic coupling between two cells leading to mass transfer. The phase difference between the chemical oscillations occurring in the two cells can be estimated by taking the difference between the nearest peak position times between two cells. FIG. 19 shows observed phase difference estimated at the peak positions between the two weakly coupled cells.

A consistent decrease in the phase difference between the two cells was observed and within the time scale of 7-8 oscillation cycles, the two cells arrived the same phase as an outcome of the weak coupling. The temporal oscillatory state of each cell is due to the time-dependent localized concentration of the reagents. Due to symmetric weak coupling, mass transfer leads to the exchange of various ionic species which eventually lead to a similar state of concentration in both cells. Once the two coupled cells arrived at the same phase, as long as the weak coupling is active, the phases of the two cells stay consistent with minor deviation over the full experimental period as evident by the FIG. 19.

The investigation was further extended by coupling three adjacent cells of the one-dimensional platform cells to demonstrate that the weakly coupling scheme could be extended over a long-range by utilizing localized neighbouring interactions. Analogous to the two-cell case, we started activating three cell stirrers with initial time shifts so that they are out-of-phase before the two interfacial stirrers get activated.

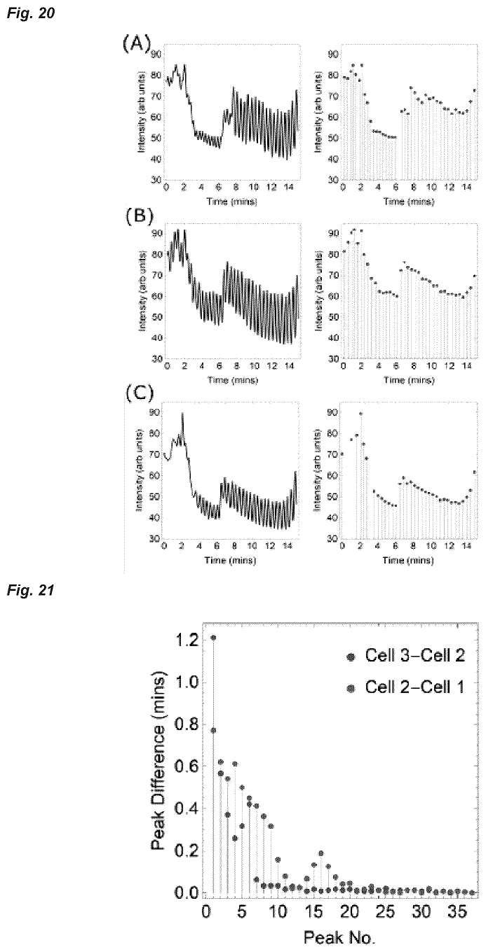

Once the two interfacial stirrers were active, the oscillations over all the three cells were recorded. FIG. 20 shows chemical oscillations and the peak positions vs. time of all the three cells. The phase differences between the central cell (cell 2) and two other two neighbouring cells (cell 1 and 3) were calculated as shown in FIG. 21. Similar to the two-cell case, all the three cells came into the same phase in around ten oscillations. This proves that in a similar way all the cells in an experiment can be bought to the same phase due to the weak coupling between neighbouring cells by activating all the interfacial stirrers. This global coupling created by weak interfacial stirring action was later used for clocking logic for hybrid decision making which is described in detail in later sections on Chemical Cellular Automata and computation.

### Image Processing

The BZ reaction is a chemical oscillator that oscillates in the analogue domain which shows a continuous transition between the red and the blue colour. Visually, it is easy to identify at least three distinct states, red, light blue and blue. A camera was placed on the top of the 3D printed reactor array to visually analyse the evolution of the oscillatory states. The video and corresponding frames captured from the camera during the experiment were processed by different image recognition algorithms which could classify the analogue signals into discretized signals with three distinct states. The temporal patterns of these discretized states were then interpreted as chemical states to define further operations throughout the experiment in a closed-loop approach. This approach of translating the analogue signal into a digital signal creates an information link between the chemical oscillations (analogue domain) and chemical states (digital equivalent) such that any Finite State Machine (FSM) can be implemented into the analogue domain via chemical states.

To characterise and recognise the colour of the oscillation waves, a “supervised learning” strategy (X features→y label) was followed. Thus, it was required to create a dataset that would relate images representing individual cells from the BZ medium in our platform (X), to three possible categories that related to their colour: “red”, “light blue” and “blue” (y).

Two databases were created. Database 1 was comprised of colour tagged images from the one-dimensional experimental setup and it contained >13,000 images. Database 2 was comprised of tagged images from the two-dimensional experimental setup and it contained >7,000 images.

The dataset was generated using user input. A researcher would load a video containing an experiment. The researcher would then stop the video at different times and click on the cells labelling them as “Red”, “Light blue”, or “Blue”. Once a cell was clicked, an RGB image was extracted from its contents, saved as Portable Network Graphics (PNG), and stored in a folder named following one of the three labels. All the data gathered for the 1D and 2D platforms underwent the same workflow.

Initially, a simple linear discriminator was tested to classify the differences between the three different classes of colour. However, during a BZ experiment, the concentration of KBrO₃ decreases over time in a non-linear way, and this dramatically changes the colour of the solution towards a more reddish shade. The ‘Red’ and ‘Light Blue’ discrete states emerging at the beginning of an experiment may look like the ‘Light Blue’ and ‘Blue’ states emerging at the middle and the later stage of the experiment, which makes them indistinguishable. Several classifiers such as Support Vector Machine (SVM), K-Nearest Neighbours (KNN), Multilayer Perceptron (MLP) and Random Forests, were tested, but none of them was able to distinguish the difference between light blue and blue reliably. To overcome this issue, Deep Learning, and in particular, Convolutional Neural Networks (CNN) was employed as this architecture has shown it can detect objects with higher accuracy than humans. Therefore, we expected that CNN would be able to classify correctly the three labels (red, light blue, and blue) throughout a full experiment.

### Convolutional Neural Network (CNN)

The input to the CNN consisted of (50,50,2) Numpy arrays. The CNN was trained using batches of size 100. The output of the CNN was a single integer value representing the class of the input array. These integer values could be 0, 1 or 2, being 0 for Blue, 1 for Light Blue and 2 for Red. The architecture of the CNN used to classify the experimental output in three different discrete states.

Tensorflow 1.X was used to define the architecture shown. Within the Tensorflow library, “Conv2D” was used as the convolutional layers. The stride was set to 1 in all of them, and the padding was set to “same”. Tensorflow “nn_max_pool” was used as the max-pool layers. When specified, the dropout was 30% (using Tensorflow layers dropout). Unless specified, the activation function is the rectified linear unit (ReLU).

The model of the CNN is as follows:
Two Conv2D with a kernel size of 9 and 64 filters
Max-pool with kernel size [1, 2, 2, 1], strides [1, 2, 2, 1].
A dropout layer.
Two Conv2D with kernel size 3 and 128 filters
Max-pool with kernel size [1, 2, 2, 1], strides [1, 2, 2, 1].
A dropout layer.
Dense layer with 64 neurons.
A dropout layer.
Dense later with 3 neurons
Softmax layer

The network was trained using Adam's optimizer. Its parameters were left to the default values. The loss function was “tensorflow.reduce_mean” paired with “minimize” from Adam's optimizer. Finally, it was trained over 1000 epochs.

### State Classification

Using the CNN, our system could visually detect three distinct colour levels of BZ oscillations in real-time: Red, Light Blue, and Blue. In order to perform logic operations, the next step was to transform this visual information into binary data, similar to the digital states 0 and 1 used in almost every electronic device. We call the three distinct colour states from the analogue oscillatory signal using the CNN as CNN states. The binary states emerging from the temporal patterns of CNN states are called as Chemical States (CS). The Chemical States defined here are the digital representation of the analogue Chemical State which exists as oscillations in the chemical domain. It is important to note that, our Chemical States do not need to be binary, it could be generalized to n distinct chemical states. However, in this work, we developed our hybrid electronic-chemical logic based on two distinct chemical states (low state: CS0 and high state: CS1). These chemical states when defined as binary states, also represented as “0” and “1” states in this section for simplicity.

The step from the three levels of visual oscillations to the digital states of CS0 and CS₁ was performed using a Finite State Machine (FSM), also referred as a recognition Finite State Machine (rFSM). It is important to distinguish the recognition FSM from the digital Finite State Machine which is used for digital processing in the Chemical Cellular Automata and Computation in later sections. Given the colour of a cell and the colour of the same cell in the next iteration, the FSM would define the chemical state of that cell as CS0 or CS1, see Scheme 2. We further introduce an accumulator, in which the CS from several clocking cycles are recorded and accumulated. This increases the robustness and minimises the error when observing the state of the cell.

Scheme 2—Finite State Machine detailing the transition rules between BZ states. The figure shows the basic implementation of the FSM to binarized chemical states where state “0” represents the chemical state CS0 and “1” represents the chemical state CS₁.

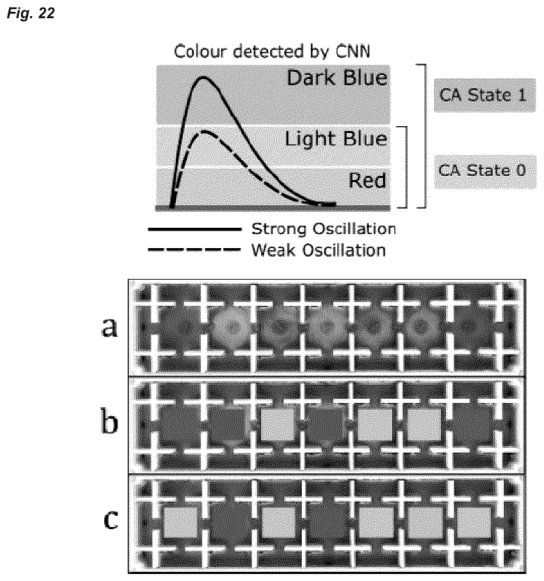

Every experiment always started with all the cells at the red BZ visual state. This is the default BZ colour when the cells are unstirred. At this colour, the binarized chemical state of the cell is defined as “0”. Once an experiment starts and the chemical medium is stirred, it will initially oscillate between the red and the light blue colours, and while the BZ medium is following this pattern, the state of each cell would be “0”. When a cell is stirred at a high speed, that cell would oscillate into blue, and only at this point the binary state of that cell would be “1”, and as soon as this cell oscillated back to red or light blue, it returned to “0”, see FIG. 22—Top. FIG. 22—Bottom shows images from the one-dimensional experiment platform of (a) image acquisition from the camera and (b) detected by the CNN in real-time, followed by (c) transformation of the detected signals to two distinct states of 1s and 0s using the described FSM.

### Mapping Between Stirrer PWM Levels and the Chemical States

We have demonstrated our BZ chemical system with programmable stirrer control act as a damped oscillator which shows strong hysteresis behaviour when the oscillations are classified as the Chemical States based on a convolutional neural network and a recognition state machine. If we assume a one-to-one mapping between PWM states and the emerging Chemical States, then at each step we should have chemical state 0 for low PWM and high chemical state 1 for high PWM. This is possible only in the absence of the hysteresis effect and weak or no interactions between the nearest neighbouring cells. To investigate both hysteresis effect and local interactions, we perform simple test experiments on a single cell and nearest neighbouring cells to demonstrate deviations from one-to-one mapping.

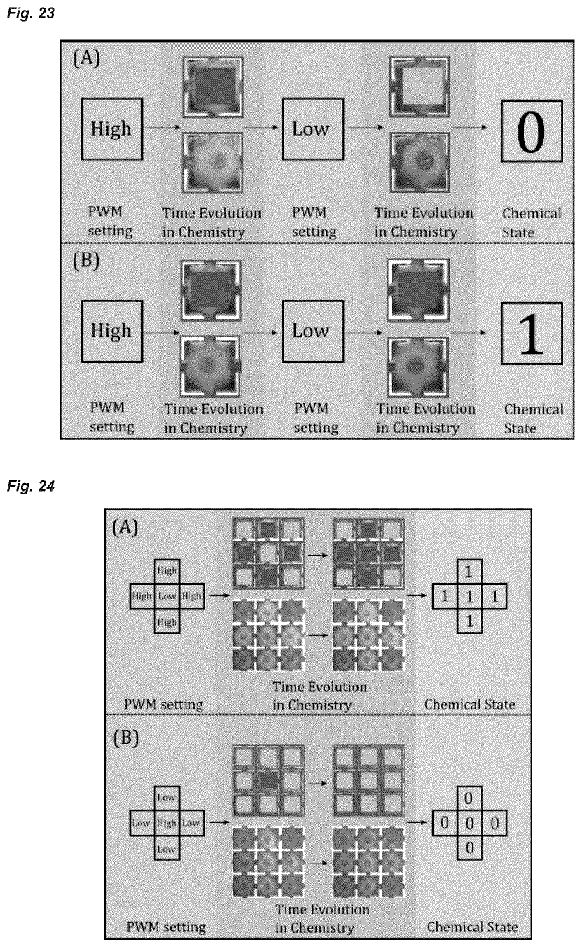

To study the hysteresis effect, we investigated the changes in the chemical states from an isolated cell. In a single cell experiment, we activated the cell stirrer at a high PWM for 2 mins so that the chemical state 1 appears. Then we switched to low PWM. If it is indeed the case of one-to-one mapping, we should expect the emergence of chemical state 0. We can observe the system decay to chemical state 0 or retains the chemical state 1, which occurs due to the damping effect from the stronger oscillation. In FIG. 23, we showed that the chemical state produced can either fade away or retain its original chemical state 1 after changing the PWM from high to low. The probabilistic mapping is more pronounced when local interactions with the nearest neighbours are introduced in the system.

To study the local interactions between cells, we further investigate the change of the chemical states when the nearest neighbouring cells are interacting with the central cells. The chemical state of the central cell is not retained and is dominated by the PWM states of the neighbouring cells instead, as shown in FIG. 24. In both cases, we activated the central cell stirrer at a high (low) PWM and the nearby cells at low (high) PWMs, and obverse the transformation of chemical state from high (low) to low (high).

### Dynamic Feedback

So far, the description on the BZ medium oscillates between different colours following the stirring patterns executed by DC motors were mentioned. A camera records the chemical oscillations based on colours, and the CNN classifies it between three different colour levels: Red, Light Blue and Blue. Finally, the state recognition FSM (rFSM), defines the chemical state of a cell as either CS0 (“0”) or CS1 (“1”) depending on the colour history of the cell. This step translates the emerging chemical oscillations (chemical domain) into equivalent chemical states on which any state machine can be implemented (digital domain). By using these current set of chemical states of all the cells, any state machine can be implemented which takes these chemical states as inputs and outputs the RPM states of the motors called as PWM states. These PWM states will update the motor speed, thus generating new oscillatory patterns. This step defines the single closed-loop operation in the context of dynamic feedback. We also defined this process as an information loop, where analogue information from the chemical oscillations accurately enters the digital domain on which various state machines could be implemented. These state machines then dynamically transfer the information back into the chemical domain. Using this approach, complex information processing is possible by distributing information within the chemical and electronic domains. Both chemistry and electronics provide different roles in the framework of information processing, which can be seen in the implementation of chemical cellular automata (CCA) and chemical entity (Chemit) in the later sections.

Here, we describe two different models of the dynamic feedback scheme which were later used in the experiments on cellular automaton and computation. Scheme 3 shows two different models of updating the chemical states in the closed-loop. In both of the models, the electronic state machine connects the chemical oscillations using via mechanical actuation of stirrers. The chemical oscillations were then recorded and processed using CNN and updates the chemical states. However, in the first case, the state machine (shown in red) as the chemical state which is defined as a cellular automata state is updated directly in-silico. This is only possible if there is a direct one-to-one mapping between the chemical state and electronic state (PWM levels of stirrers in our case) and all the individual chemical oscillations directly translate back into the chemical states. In this case, there are no interactions between the analogue chemical states. The exact information from the digital states loops through the analogue domain without providing any additional benefit towards computation using hybrid electronic-chemical logic. We call this implementation as a “display screen” due to direct mapping to the digital domain.

Scheme 3—Two models of the dynamic-feedback loop. (a) Shows that the FSM logic is embedded in silico such that the new state can be predicted within the digital domain and (b) on the other hand shows that the FSM logic is based on the chemical system.

In the second case, the state machine shown in red reads the chemical states and applies state machine logic to the PWM states of the stirrers, which is shown as an electronic state. In this scenario, it is not possible to predict the evolution of chemical oscillations in the analogue domain and the emerging chemical states. So, there is no direct one-to-one mapping between the chemical states to the digital states. This dynamic loop model describes the true picture where the information processing operations can be split into chemical and digital domains. In the chemical domain, analogue information processing occurs due to interactions between the cells with different oscillatory states, and digital information processing occurs using state machines utilizing the chemical states of the information loop. We have implemented both states machines, the first model to demonstrate successful implementation information loops over the complete experimental time scale by implementing elementary Cellular Automata (ECA). The second model was used to further develop novel one-dimensional chemical cellular automata rules (1D-CCA), two-dimensional chemical cellular automata (2D-CCA) implementation, and computation experiments. In the next step, we introduce the concept of chemical clocking logic which is critical towards implementing logic to develop hybrid electronic-chemical decision-making machine.

### Synchronization Between the Computing Cells Using a Chemical Clock

As the BZ chemical medium starts oscillating in our experimental platform, we observed a minor phase shift between the different cells due to hysteresis effects of the oscillating reaction and possibly a little stochasticity associated to the stirring actuation signals. Thus, there is a finite phase difference between the oscillations across the whole platform. To get rid of this initial phase difference and not creating strong oscillations which could distort the actual signals, we used the strategy discussed in Section 2.5.2. From our observations as discussed previously, if interfacial stirrers are active, the neighbouring cells come into the same phase and oscillate together. To fix the phase drifting problem, we activate the interfacial stirrers so that all the cells can interact with nearest neighbours and global phase synchronicity can be achieved. Instead of activating the cell stirrers all the time, we pulse them between ON and OFF states. This creates a weak but detectable in-phase oscillation over all the cells. Instead of using no oscillation state as the low state (equivalent to chemical state CS0), we use the weak oscillatory state as chemical state CS₀.

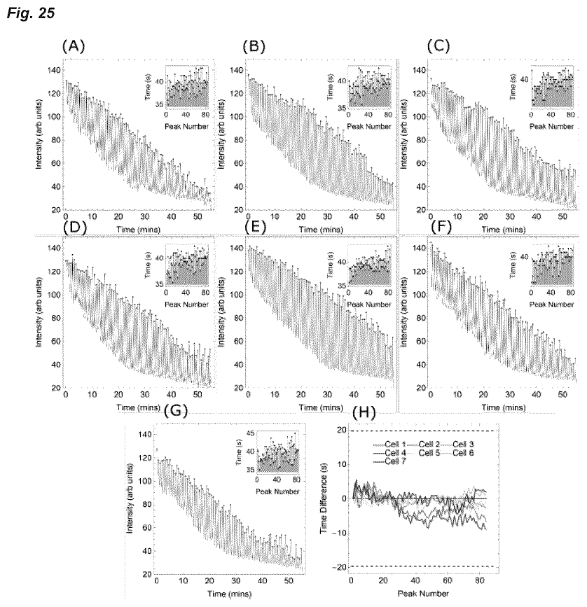

FIG. 25(A-G) shows BZ oscillations over all the seven cells of the one-dimensional array with peak positions highlighted with interfacial stirrers active and pulsing cell stirrers. FIG. 25 H shows the time difference of the oscillations occurring at the seven cells of the 1D platform throughout a full experiment when compared against the central cell. We observed an increase in the phase difference as the experiment progresses, up to 10 seconds at the end of the experiment. However, as the difference between the consecutive peaks is ca. 40 seconds, the phase drift of 10 seconds does not pose any problem to implement any programmable logic into the system.

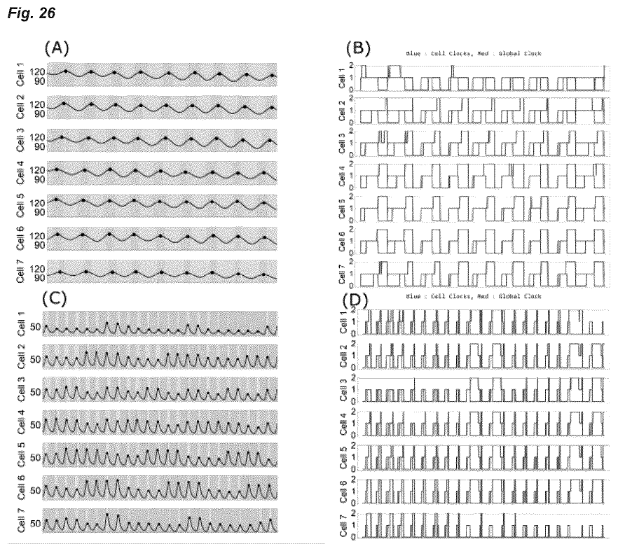

To perform computations, we define windows of time where all the BZ cells oscillate are needed, despite the drift of oscillations over time. Initially, it was decided to set a fixed quantity of time for each window, based on the average periodicity of the oscillations, but as the temporal phase between oscillations drifted through time, this solution was not suitable. Therefore, it was decided to dynamically alter the windows of computation based on the chemical oscillations. This was implemented similar to the clock element used in digital computers. In a digital computer, when the clock signal goes from 0 to 1 it is often called “tick” and when it goes from 1 to 0 it is often called “tock”. Usually, computation only happens during the “tick” phase—that is, when the clock is set to 1. In digital computers, the clock signal is perfectly periodical with a square shape—a 1:1 ratio between tick and tock—while in our case, as it can be seen on FIG. 26(A, C), the portion of time when the BZ medium did not oscillate was much bigger than the portion of time it oscillated.

Following this idea, it was decided to define a “chemical clock” that would track the BZ oscillations. This clock would be 0 when no oscillations happened, and it would be 1 while the BZ medium oscillated. Because each cell could oscillate individually, there were two types of chemical clocks defined: local clocks which represented each of the individual clocks, and a global clock signal, which represented how the BZ medium oscillated as a whole, and it was calculated based on the local clocks.

Each local chemical clock could have three states:
1. None—which meant the medium was not oscillating, i.e. the CNN classified the medium as red colour.
2. Tick—which meant the BZ medium went from red to blue.
3. Tock—which meant the BZ medium went from blue to red.

There were three states defined for the global chemical clock:
1. None—which meant the medium was not oscillating: all the local clocks were in the “none” state.
2. Tick—which meant at least one cell oscillated: at least one local clock went into “tick” state.
3. Tock—which meant all the cells had oscillated: all the local clocks were into “tock” state.

Once a global Tock signal was executed, all the calculations related to the computations using the BZ state would be performed, and the global clock would reset and go back to “None”, and it would also set to “None” all the local clocks, see Scheme 4 for schematic diagram. The transitions between these three states were as follows:
1. To Tick—Once the first local clock “tick” happened (red to blue), the global chemical clock signal went from none to tick.
2. To Tock—Once all the local clocks were in “tock”, the global clock signal went from tick to tock.
3. To none—Once the computations related to the BZ states were performed, the clock signal went from tock to none.

In the case of the 1D platform, this workflow worked perfectly well, because it was only required that 7 cells would fully go through an oscillation. In the case of the 2D platforms these conditions had to be loosened, this is because the 49 cells may not oscillate in a similar period. Therefore, in the case of the 2D platform the To Tock step was as follows:
2. To Tock—Once at least 15 cells have oscillated—thus their local clock was on the tock state—and all of the 49 cells were back in red colour, the clock signal went from tick to tock.

To perform a computation based on the states described by the BZ medium, it was required that the chemical clock signal fully oscillated twice before a decision was taken. This was done to improve the reliability of the chemical state as acquired by the camera and minimise any error that could arise from the phase-drift of the oscillations.

A simplified analysis was performed on clocking mechanism after experiments were complete for representing local clock for individual cells and global clocking scheme for basic clocking test and tests performed with dynamic loops to implement elementary cellular automata (see later sections). The chemical clocking logic was observed as
1. The initial state is when all cells are red.
2. Once a cell oscillates (it only needs one cell), the cell goes to TICK, and global clock also goes to TICK.
3. Once the cell stops oscillating (going from blue to red), the cell goes to TOCK and global clock tests if global TOCK is possible.
4. Global TOCK is only possible if all the seven cells are red and at least two of them are in TOCK.
5. After global TOCK the clock resets.

The condition for every decision to be taken place is that the global clock is required to ‘tock’ twice, this further improves the reliability of the chemical state acquired by the camera.

### Implementation of Elementary Cellular Automata

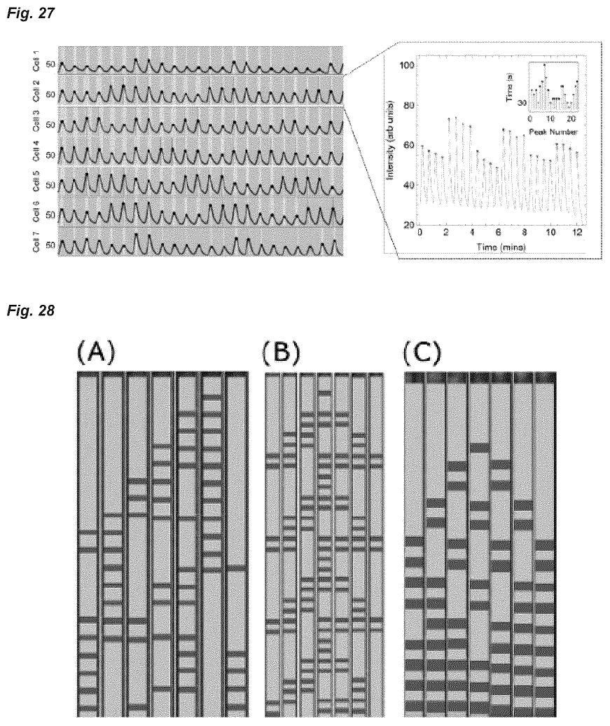

To demonstrate the successful implementation of the dynamic feedback loop of the information transfer back and forth in chemical and digital domains, experimentally, we implemented three elementary cellular automata rules, Rule 30, 110 and 250 in the one-dimensional experimental setup. In this case, we have a direct one-to-one mapping between the PWM state of the cell stirrer and emerging chemical state. In brief, all the interfacial stirrers were turned on to allow coupling between the cells and their nearest neighbouring cells. The cell stirrers can either be CS₀ achieved by constant pulsing (activate stirrer at 16 PWM for 5 seconds and deactivate of stirrer for 15 seconds) and CS₁ achieved by strong stirring operations (activate stirrer at 50 PWM). We implemented these rules to demonstrate successful information propagation from digital logic to analogue oscillatory chemical oscillations which read back into the electronic state via optical imaging and discretization using Convolutional Neural Networks (CNN) as discussed in previous sections. The basic strategy was shown in Scheme 3(a). A detailed description of the BZ oscillations and the CNN states emerging in the elementary CA rule 30 in the one-dimensional experimental setup is shown in FIG. 27. The intensity versus time plot of cell 2 indicated clearly for the lower intensity oscillations, chemical state CS₀ achieved by basic pulsing operation of the cell stirrers and the higher intensity oscillation, chemical state CS₁ strong stirring operations. As seen in the figure, the period of an oscillation varied from 30 seconds to 40 seconds.

The chemical states of emerging rules in elementary CA rules 110, 30 and 250 are shown in

FIG. 28 with chemical states, CS₀ shown in light blue and CS₁ shown in dark blue. All the three implemented rules implemented as chemical states mapped perfectly with the in-silico elementary CA rules. As discussed in the dynamic feedback model, elementary CA rules can be predicted in-silico, with the direct one-to-one mapping as the information loops through chemical and electronic domains. To bypass this limitation, we updated the implementation of elementary CA rules by introducing asymmetric coupling between the nearest neighbouring cells to create novel rules which cannot be predicted directly.

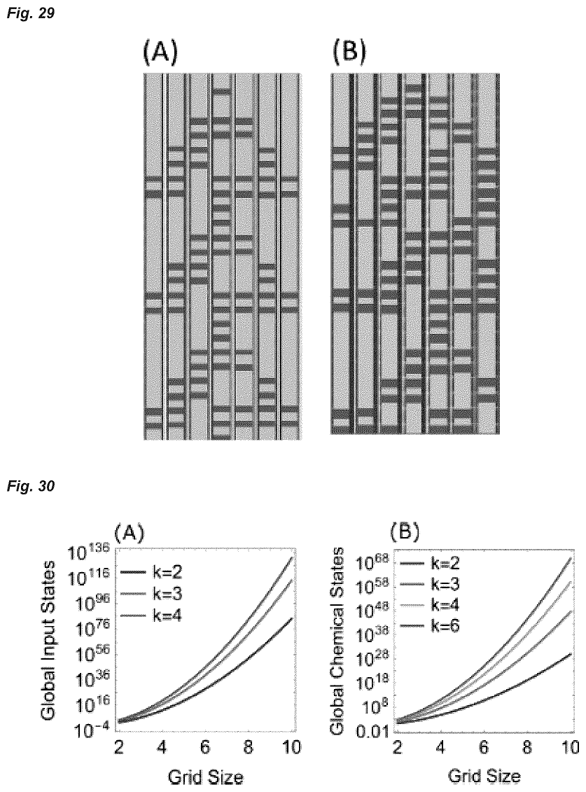

We also extended the definition of the elementary CA and implemented novel CA rules using our experimental platform. These novel rules are defined as 1D-CCA rule, where we use the dynamic feedback loop scheme as shown in Scheme 3(b), see full implementation details in the next section. The novel rules emerge by expanding the state space using chemical states as well as PWM states. FIG. 29(A) shows an experimental implementation of elementary CA rule 30 and (B) shows modified 1D-CCA rule with novel states emerging due to asymmetric interactions between nearest neighbouring cells. In the logic of the asymmetric variation, the interface is only turned on when the left neighbour is at CS1 whilst the cell is at CS₀.

### 1D-CCA Phenomenological Model

The phenomenological model is a probabilistic model which updates the chemical state of 1D-CCA based on probabilities assigned to various combinations of PWMs of cell and interfacial stirrers. In the simulation, the time-stepping of the chemical states occurs using two state machines, digital (DD) and chemical (CC) which defines the 1D-CCA rule and phenomenological model respectively. Considering SSiitt is PWM state of the ith cell stirrer, IIii,jjtt as PWM state of interfacial stirrer between the ith and jth cells and CCCCiitt as the chemical state of the ith cell, the state machines are represented as,

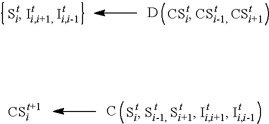

### Benchmarking Elementary CA States Vs 1D-CCA States

The total number of possible states for the N×N array is 2^{N,N} for both elementary CA and 1D-CCA. But while in elementary CA rules the number of explored states is limited by the possible cell patterns of the given neighbourhood, in 1D-CCA the state space is expanded by the hidden PWM states of cell and interfacial stirrers which leads to the probabilistic outcomes. As an example, for a given elementary CA rule, cell state (CS^{t}ᵢⱼ) and its neighbours (CS^{t}ₘₙ), there is a single new state. For 1D-CCA, for a given CA rule, cell state (CS^{t}ᵢⱼ) and its neighbours (CS^{t}ₘₙ), multiple novel states are possible depending on state transitions on PWM states of cells and interfacial stirrers (S^{t}ᵢⱼ, S^{t}ₘₙ, I^{t}_{ij,mn}) with programmable probabilistic effects as shown by the 1D-CCA rules. The evolution of the new 1D-CCA states occurs due to a strongly non-linear coupling between the chemical kinetics, hysteresis and hydrodynamic effects; hence a very large number of internal digital states are required to emulate these coupled physical phenomena and hence the chemical system is much less computationally expensive.

### Estimation of BZ Input and Chemical State Space

Consider an experimental set up with n×n interconnected network of cells, with cell and interfacial stirrers which can be addressed independently. If we assume possible cell stirrer states and possible interfacial stirrer states, the total number of possible combinations (Input States: IS) are given by IS=p^{n·n}q²ⁿ⁽ⁿ⁻¹⁾. The first term p^{n·n} corresponds to all the operations on the cell stirrers where · corresponds to the total number of stirrers in the two-dimensional experimental platform. The second term q²ⁿ⁽ⁿ⁻¹⁾ corresponds a total number of possible combinations of interfacial stirrers which are present in both horizontal and vertical connections. All combinations of cell stirrer and interfacial stirrer states are a subset of the complete input space. Similar to the input space, we can also estimate the total number of chemical states which can be interpreted from the chemical oscillation patterns by classification using CNN as discussed in previous sections. Assuming, different possible chemical states on each cell, the total number of possible global chemical states is given by CS=k^{n·n}.

Depending on the type of operations on our computational platform, we explore through the subset of this input space. Various operations include Chemical Cellular Automata, solving NP-hard problems based on chemical computation logic etc. Each experimental rule or logic creates a trajectory in both input and chemical state space. FIG. 30 shows scaling of total input and chemical states for a two-dimensional set up with the number of cells.

For the implemented computational architecture with 7×7 array with four distinct PWM states for cell stirrers (as used in two-dimensional chemical cellular automata) and two PWM states for interfacial stirrers, the total number of input states are given by 4^{7×7}2^{14×6}=6.12×10⁵⁴. The total number of possible chemical states are given by 2^{7×7}=5.6×10¹⁴.

In the presence of one-to-one mapping between the input and chemical states, the total number of input and chemical states are equal. Hence, the information loops between input and chemical states without any additional benefit towards efficient computation. However, in the current case with many-to-many mapping and for the given initial chemical state, same new chemical state can be observed with various input state configuration in a probabilistic manner. These novel paths leading to the same chemical state is an outcome of the state expansion in the hybrid electronic-chemical probabilistic computation. In the one- and two-dimensional chemical cellular automata and computation as described in the later sections, the hybrid chemical-electronic state machine uses these probabilistic pathways for the novel computation. This state expansion of many-to-many as compared to one-to-one mappings can be simply quantified by the ratio of input states (many-to-many) and chemical states (equivalent to input states in one-to-one) which is given by p^{n·n}q²ⁿ⁽ⁿ⁻¹⁾k^{-n·n}. For the n×n array, with two possible PWM levels for each central and interfacial stirrer such that p,q,k=2, the state expansion ratio is 2⁸⁴=1.9×10²⁵.

### Simple Quantification of Trajectory in State Space Using Jaccard Dissimilarity

Considering one-dimensional Chemical Cellular Automata with two possible chemical states (0/1) with n connected cells. At each step, the current set of chemical states can be defined using a one-dimensional Boolean vector. As the size of the sample set is finite, we can quantify the difference between the Boolean vectors at two consecutive time steps (and +1) using the Jaccard dissimilarity also called as Jaccard distance. The Jaccard dissimilarity between two sets and is given by,

$${d}_{j} = 1 - J ( A , B ) = \frac{\vert A ⋃ B \vert - \vert A ⋂ B \vert}{\vert A ⋃ B \vert}$$

where J(A,B) is the Jaccard index defined as Intersection over Union. For two Boolean vectors, the Jaccard index can be defined as

$${d}_{j} = \frac{{M}_{01} + {M}_{10}}{{M}_{01} + {M}_{10} + {M}_{11}}$$

By quantifying the Jaccard dissimilarity, for a given Chemical Cellular Automata rule, we can represent the CCA trajectory in the state space defined all combinations of state space as described in the previous section. As an example, consider a one-dimensional space with n cells and two possible discrete states (0/1) for each cell. The total number of possible bit sequences is given by 2ⁿ. A single CCA rule represents a trajectory through different 16 possible states (e.g. a trajectory sequence for a CCA rule could look like {3→4→7→2→8→11→14} for 7 steps, where the number is the node index) (index not shown).

We estimated the Jaccard Index between two consecutive steps on Elementary Cellular Automata rules to observe the difference between the trajectories in the state space defined by different CA rules.

We observe different trends in the Jaccard index for different rules, as rule 250 the difference between consecutive states stays constant due to alternate emerging patterns (index not shown). Rules 30 and 110 shows strong variations initially and converges towards specific values and rule 45 shows a monotonic decrease with an increase in the number of time steps.

### Phenomenological Model for 1D Chemical Cellular Automata Rules

Inspired from the elementary CA rules, we also developed a family of One-dimensional Chemical Cellular Automata (1D-CCA) rules based on a closed-loop state machine utilizing PWM and chemical states. As described in the previous section, a CCA rule can be defined on an experimental platform where based on the observed chemical states, PWM levels of interfacial and cell stirrers selected. In the absence of interfacial stirrers, we can recreate elementary CA rules within a closed feedback loop due to one-to-one mapping between PWM states of cell stirrers and observed chemical states. However, when the interfacial stirrers are active, due to hydrodynamic coupling between the neighbouring cells and coupled hysteresis effects, one-to-one mapping between PWM and chemical states does not exist and novel patterns can emerge out.

Here, we describe the 1D-CCA state machines and phenomenological model which was then used to simulate the emergence of patterns in a one-dimensional geometry. 1D-CCA rules which define the state machine to act on stirrer based on chemical states comprises of two different values, {Rule_A}-{Rule_B}. Rule_A updates the central cell stirrer PWM state based on chemical states of nearest neighbouring cells (C^{t}ᵢ, C^{t}ᵢ₋₁, C^{t}ᵢ₊₁) similar to the elementary CA rule table. Rule_B updates the PWM states of the two interfacial stirrers based on the chemical states of the two connecting cells (−1,) and (, +1). Under this scheme, there are 2³=8 possible chemical states for three neighbouring cells. Similarly, to the elementary CA rules, there are 2⁸=256 possible rules for Rule_A. There are 2² possible chemical states for two neighbouring connecting cells at the interfacial stirrer, hence total 2²2²=16 rules are possible for Rule_B for both interfacial stirrer. The total number of 1D-CCA rules possible is 2⁸2⁴=4096.

To simulate the 1D-CCA, we used a simple phenomenological model which updates the chemical states of all the cells based on the PWM values of cell and interfacial stirrers. Here, we define S^{t}ᵢ as the PWM state of the cell stirrer, I^{t}_{i,i+1} as the PWM state of interfacial stirrer between i^{th} and I=1^{th} cells. Here, we define the chemical state of the i^{th} cell is given by CS^{t+1}ᵢ. Assuming two different PWM states for both cell and interfacial stirrers, and two different chemical states, we can define the probability for the chemical states purely based on PWM levels of cells and interfacial stirrers. The probabilities for the high chemical state (1) at different conditions are,

$$\{ \begin{matrix}{S}_{i}^{t} == 1 and {S}_{i - 1}^{t} == 1 / 0 and {S}_{i + 1}^{t} == 1 / 0 and \\ {I}_{i , i + 1}^{t} == 1 / 0 and {I}_{i , i - 1}^{t} == 1 / 0\end{matrix} \} : {CS}_{i}^{t + 1} \to 𝒫 ( 1 ) = 1$$

$$\{ \begin{matrix}{S}_{i}^{t} == 0 and {S}_{i - 1}^{t} == 0 and {S}_{i + 1}^{t} == 0 and \\ {I}_{i , i + 1}^{t} == 1 / 0 and {I}_{i , i - 1}^{t} == 1 / 0\end{matrix} \} : {CS}_{i}^{t + 1} \to 𝒫 ( 1 ) = 0$$

$$\{ \begin{matrix}{S}_{i}^{t} == 0 and {S}_{i - 1}^{t} == 1 / 0 and {S}_{i + 1}^{t} == 1 / 0 and \\ {I}_{i , i + 1}^{t} == 0 and {I}_{i , i - 1}^{t} == 0\end{matrix} \} : {CS}_{i}^{t + 1} \to 𝒫 ( 1 ) = 0$$

$$\{ \begin{matrix}{S}_{i}^{t} == 0 and {S}_{i - 1}^{t} == 1 and {S}_{i + 1}^{t} == 1 and \\ {I}_{i , i + 1}^{t} == 1 and {I}_{i , i - 1}^{t} == 1\end{matrix} \} : {CS}_{i}^{t + 1} \to 𝒫 ( 1 ) = 0.8$$

$$\{ \begin{matrix}{S}_{i}^{t} == 0 and {S}_{i - 1}^{t} == 1 and {S}_{i + 1}^{t} == 1 / 0 and \\ {I}_{i , i + 1}^{t} == 0 and {I}_{i , i - 1}^{t} == 1\end{matrix} \} : {CS}_{i}^{t + 1} \to 𝒫 ( 1 ) = 0.5$$

$$\{ \begin{matrix}{S}_{i}^{t} == 0 and {S}_{i - 1}^{t} == 1 / 0 and {S}_{i + 1}^{t} == 1 and \\ {I}_{i , i + 1}^{t} == 1 and {I}_{i , i - 1}^{t} == 0\end{matrix} \} : {CS}_{i}^{t + 1} \to 𝒫 ( 1 ) = 0.5$$

$$\{ \begin{matrix}{S}_{i}^{t} == 0 , {S}_{i - 1}^{t} == 1 , {S}_{i + 1}^{t} == 0 , \\ {I}_{i , i + 1}^{t} == 1 / 0 , {I}_{i , i - 1}^{t} == 1\end{matrix} \} : {CS}_{i}^{t + 1} \to 𝒫 ( 1 ) = 0.5$$

$$\{ \begin{matrix}{S}_{i}^{t} == 0 , {S}_{i - 1}^{t} == 0 , {S}_{i + 1}^{t} == 1 , \\ {I}_{i , i + 1}^{t} == 1 , {I}_{i , i - 1}^{t} == 1 / 0\end{matrix} \} : {CS}_{i}^{t + 1} \to 𝒫 ( 1 ) = 0.5$$

These probabilities were assigned based on our knowledge on how the experimental setup behaves on the actuation of cell and interfacial stirrers. These probabilities were assigned based on the expected outcome such as, in the first case if the cell stirrer of the central cell is active (PWM state S^{t}ᵢ=1) independent of the other controls, the probability of the occurrence of the high chemical state is 1 as due to the stirrer action the chemical state of the cell will be high. If the central cell stirrer is inactive, however, both the neighbouring cells and the interfacial stirrers between them are active (see condition 4 above), the probability of occurrence of the high chemical state was chosen to be 0.8. This high probability is due to the combined interaction from both neighbours together when the interfacial stirrers are also active.

Similarly, instead of two neighbouring cells if only one neighbouring cell stirrer is active together with the active interfacial stirrers, the probability for the occurrence of high chemical state was chosen to be 0.5. It is important to note that, these assign probabilities are based on the certain values of PWM levels of cells and interfacial stirrers. We can tune these probabilities by selecting another set of PWM values.

The single step of the simulation consists of running both 1D-CCA state machine for a given rule and phenomenological state machine to update the new chemical states as described by the two-state machines D and C.

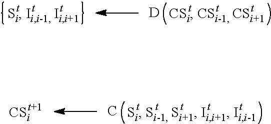

where D is the 1D-CCA rule and C is the probabilistic state machine as defined above. We implemented the 1D-CCA simulation in Mathematica 12 (Wolfram Ltd.) where based on the probabilities, new chemical states are calculated using random choice between the states (0/1) based on weights defined by the probabilities. An example code for 1D-CCA is available on GitHub. The simulation of 1D-CCA rule 30-{i} with i∈[1, 16], where the first rule is similar to elementary CA rule 30 shows the perfect one-to-one mapping between chemical states and PWM states of cell stirrers (simulations not shown). Significant deviations from the basic rule can be observed by introducing the effect of interfacial stirrers, which gives rise to new behaviours. However, it is important to mention that 1D-CCA rules are not completely deterministic. Due to strong hysteresis effects and hydrodynamic coupling, there is associated stochasticity for switching between chemical states. 1D-CCA allows us to program the stochastic effects in the system by selecting PWM levels for cell stirrers as well as interfacial stirrers. We have also demonstrated an experimental example of the emergence of new chemical states by running elementary cellular automata rule 30 with symmetric and antisymmetric couplings.

To quantify the differences between elementary CA rules and 1D-CCA rules, we used Jaccard index between the CA rule N−1 and rules N−i∈[2, 16], at each time step, where N is the Rule_A. Fig. S53 shows the Jaccard index calculated at each time step (up to 100 steps) between 1D-CCA rule 30-1 and rest of the rules of 30 families (30-{i} with i∈[1, 16]).

### Two-Dimensional CCA Simulation

The phenomenological model does not consider the oscillatory BZ dynamics and the complex hydrodynamic interactions to update the new state, instead, it updates directly the chemical state (CS₀/CS₁) based on defined event probabilities. The simulation uses the same digital state machine and utilizes a probabilistic model to update the chemical states as an outcome of the updated PWM levels. The phenomenological model uses a combination of PWM states of the central and the four neighbouring cells to calculate the probability of occurrence of new chemical states. Each time step of the simulation occurs in two steps, estimating the new PWM from the current chemical states using a two-dimensional CCA state machine DD which then updates the new chemical state using a phenomenological model CC,

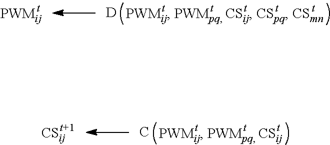

where ij, pq, mn are the indices of the central cell, nearest-neighbouring cells and next-nearest neighbouring cells. A detailed description of the implementation of the probabilistic model and simulations are given in the SI. A further data analysis was performed from the time series data of PWM and chemical states to evaluate population and propagation dynamics.

### The Chemits: 2D Hybrid Electronic Chemical Automata

Two-Dimensional Chemical Cellular Automata (2D-CCA) is a zero-player two-dimensional Cellular Automata with chemical entities (Chemits) driven by the Chemical Cellular Automata (CCA), which are triggered by chemical oscillations. The key feature is that the Chemit is extended and comprised of 5 cells, with one central cell and its four neighbours. The central cell is the Chemit's core which is essential for existence. The four surrounding cells are used to interact with the fluctuating environment. These cells are not crucial for the existence of the Chemit and they can disappear and appear again during propagation, replication and competition events.

In the 2D-CCA experiments, we used four different PWM levels of the cell stirrers defined as PWM₁=0 (maximum PWM value is 255) for no interaction such that chemical state of the cell returns to the lower state, PWM²=22 to create random fluctuations in the environment as BZ chemical oscillator is a damped oscillator, PWM³=50 to create the core cell of the Chemit, PWM⁴=30 for the nearest neighbours of the core cell of the Chemit which is used for interaction with other Chemits and random fluctuations. The experimental loop consists of applying PWM states to the cells together with clocking logic, readout the chemical states as described in the previous sections. We use the 2D-CCA State Machine to update the new PWM states of all the cell stirrers.

### Phenomenological Model

To simulate the dynamics of the Chemits emerging in the 2D-CCA, we developed a simple model of the evolution of chemical states based on the PWM actuation of the cell stirrers. We avoid details of physical models describing hydrodynamic interactions between and cells and its coupling to BZ oscillator kinetics. Computational models of BZ reaction coupled with hydrodynamic interactions are extremely complex to simulate and computationally intensive. Our phenomenological model comprises of two different state machines,
1. 2D-CCA State Machine (D): Same state machine as used in experiments which read in chemical states and update the PWM states.
2. Phenomenological State Machine (C): Based on the observed phenomena in 2D-CCA experiments, it reads in PWM states and previous chemical states and outputs the new chemical states.

The time-stepping the simulation occurs in two steps, at each step first the new PWM states of all the cells were calculated from the state machine D,

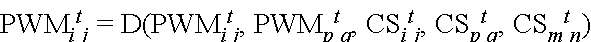

where CS^{t}ᵢⱼ is the chemical state of ij^{th} cell, CS^{t}_{pq}, CS^{t}ₘₙ are the chemical states of the nearest and next-nearest neighbours and PWM^{t}ᵢⱼ, PWM^{t}_{pq}, are the PWM states of the central and the neighbouring cells. Once the PWM states were updated, new chemical states were calculated from the second phenomenological state machine C,

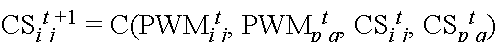

where CS^{t}ᵢⱼ defines the chemical state of the ij^{th} cell and CS^{t}_{pq}, CS^{t}ₘₙ describes of

chemical states of nearest neighbours and the next-nearest neighbours. To describe the state machine C, we define a list of probabilities for switching chemical states for different scenarios of previous chemical states and the applied PWM levels. Out of the four different PWM levels, for nearest neighbouring interactions, we only consider the effect of PWM₃ and PWM₄.

nPWMj: Number of neighbouring cells with PWM value j.

If (nPWM₃ ≥ 3): Cᵢⱼ = P₁
Else If (nPWM₃ ≥ 1 and nPWM₃ < 3): Cᵢⱼ = P₂
Else If (nPWM₄ ≥ 3 and nPWM₃ == 0): Cᵢⱼ = P₃
Else If (nPWM₄ ≥ 1 and nPWM₁ ≤ 3 and nPWM₃): Cᵢⱼ = P₄
Else: Cᵢⱼ = 0

We defined additional parameter based on the PWM state of the central cell given by Q which can take three different values {Q1, Q2, Q4} for PWM level of the central cell {PWM₁, PWM₂, PWM₄} respectively. When the PWM of the central cell is PWM₃, in that case, there is a high probability that the next chemical state will be High (1), so the effect of neighbours is irrelevant. We also introduced an additional factor K which introduces the effect of the current chemical state due to the hysteresis effect, whether CS^{t}ᵢⱼ=0 or 1, we defined K for ij^{th} cell as,

$${K}_{ij} = \{ \begin{matrix}0.7 & if {CS}_{ij}^{t} == 0 \\ 1 & if {CS}_{ij}^{t} == 1\end{matrix}$$

Using these parameters, for a given cell the probability of the high chemical state (1) is given by,

$${P}_{ij} ( 1 ) = {K}_{ij} \{ \begin{matrix}{Q}_{1} {C}_{ij} & if {PWM}_{ij} == 1 \\ {Q}_{2} {C}_{ij} & if {PWM}_{ij} == 2 \\ {Q}_{3} & if {PWM}_{ij} == 3 \\ {Q}_{4} {C}_{ij} & if {PWM}_{ij} == 4\end{matrix}$$

By estimating the probabilities for all the cells, new chemical states were updated by randomly selecting chemical states {0/1} based on weights given by {1−Pᵢⱼ(1), Pᵢⱼ(1)}. The simulation requires eight different variables for estimating the probabilities {P₁, P₂, P₃, P₄, Q₁, Q₂, Q₃, Q₄, K}. The simulations were performed in Mathematica 12 (Wolfram Ltd.) with basic implementation and source code available on <https://github.com/croningp/BZComputation>.

The simulation parameters we chose are,

$$\{ {P}_{1} = 0.5 , {P}_{2} = 0.3 , {P}_{3} = 0.25 , \\ {P}_{4} = 0.1 , {Q}_{1} = 0. , {Q}_{2} = 0.1 , {Q}_{3} = 0.5 , {Q}_{4} = 0.5 \}$$

The simulation parameters were chosen similarly as described in the phenomenological model for one-dimensional CCA. As discussed previously, the probabilities can be easily modified by selecting different PWM levels

As a first step, we tested the outcomes 2D-CCA state machine to test the emergence of propagation, replication and competition events. We created the initial Chemit defined by PWM values PWM₃ (core) and PWM₄ (interacting nearest neighbours) on 5×5 grid. We created the chemical states ourselves to monitor the emergence of new PWM states showing the expected behaviour.

### Propagation Event

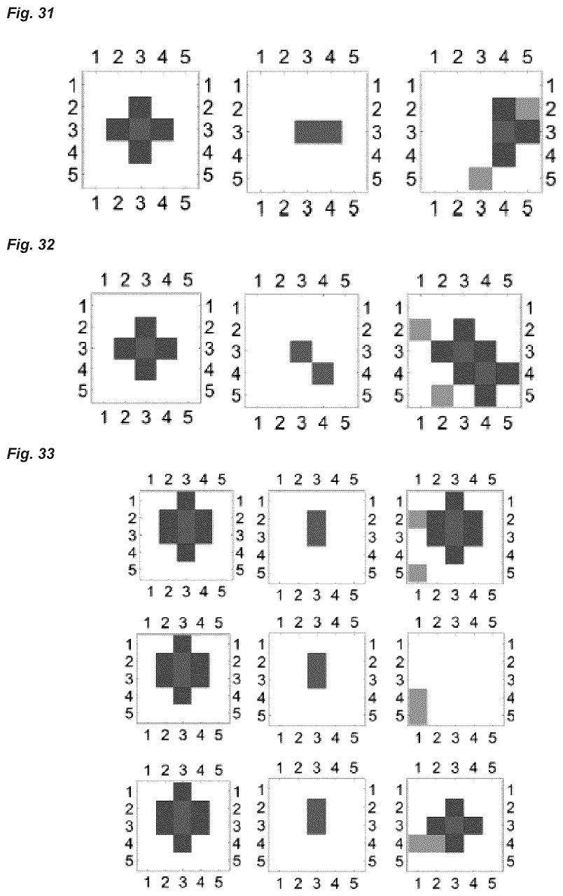

As a first example, to demonstrate the chemical state we created a high chemical state at one of the nearest neighbours (right cell) as shown in FIG. 31. Using the old PWM states and the chemical states, we used the 2D-CCA state machine as described above and create the new PWM states. As shown in the figure, the Chemit propagated in the expected direction. It is important to note that the position of the Chemit can be precisely defined by the position of PWM₃ core cell. The surrounding interaction cells can appear or disappear and are not considered as a necessary feature of the Chemit. In the right figure, the core cell of the Chemit propagated to right with some additional random fluctuations (PWM₂) occurring as an outcome of the 2D-CCA state machine.

### Replication Event

Similar to the propagation event, with initial PWM levels describing the position of the Chemit, we created the high chemical state at one of the next nearest neighbour (bottom right) and used the 2D-CCA state machine to get the new PWM state as shown in FIG. 32. In our construction of state machine, we chose replication events possible when the chemical state at the next nearest neighbour is high to limit the replication events relative to propagation events. We observed replication event as expected in the 5×5 grid, see FIG. 32.

### Competition Events

When the density of Chemits increases due to the replication event, two Chemits can meet with each other and a competition event may happen. In the competition event, the Chemit observes a high chemical state which is already the core of another Chemit and interact with it. There is a 50% chance to survive. Since we loop through all the Chemits, if two Chemits are competing with each other, the probability for (i) both of them survive, (ii) both of them die and (iii) one of them survives are 25%, 25% and 50% respectively. FIG. 33 shows three different scenarios, in the first case both Chemits survive, in the second case both Chemits annihilate each other, and in the third case a single Chemit survives.

### Random Selection Among Multiple Events

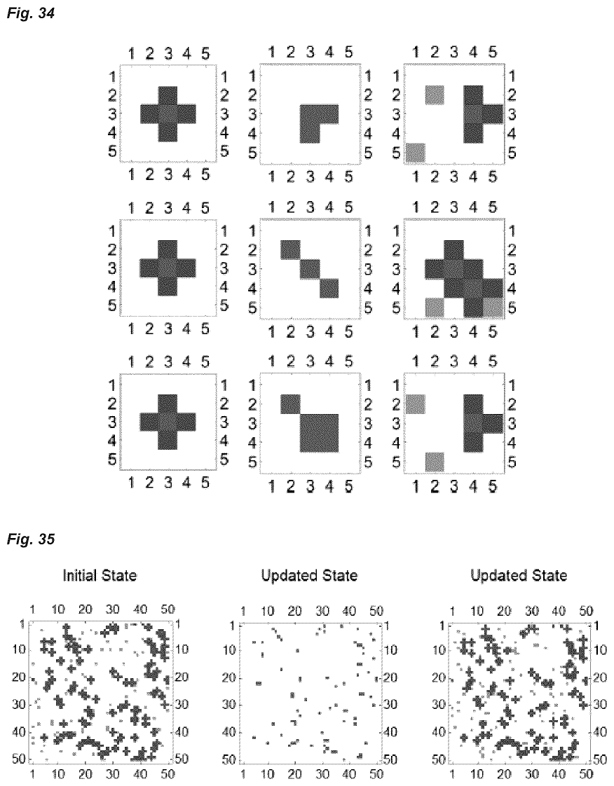

In the propagation and replication events, only one of the nearest or the next nearest neighbour was at high chemical state. However, in the experiments, we observed the occurrence of multiple chemical high chemical states simultaneously due to strong coupling among the nearest neighbours and hysteresis in chemical oscillations. In this case, among all the high chemical states, we randomly chose one cell among nearest and next-nearest neighbours, which define competition between possible propagation and replication events. FIG. 34 shows three different examples of multiple events showing propagation in the first and the third case, replication in the second case.

As a next step, we tested the simulation step by coupling phenomenological state machine together with 2D-CCA state machine as shown in FIG. 35. For a single step, we start with initial PWM states created using randomly placed Chemits over 50×50 array. FIG. 35 (left) shows all four PWM states present in the initial state. Using the phenomenological state machine, we updated the chemical states which are shown in FIG. 35 (middle). It is easy to observe a high density of chemical states at the positions of PWM₃ core cells and their neighbours. As a second step, we update the PWM states from the chemical states using 2D-CCA state machine as shown in FIG. 35 (right). We used two-dimensional Periodic Boundary Conditions (PBC) for the Chemits if they cross the boundary cells. Comparing the initial and final PWM states, we can observe propagation, replication and competition events at a different location on the two-dimensional grid.

### Simulations and Results

Using the simulation scheme as described in the previous subsection, we ran simulations over 100×100 grid and up to 15,000 simulation steps with various initial conditions and parameters. Due to the stochastic nature of the simulations, we ran the same simulation 25 times and estimated the average properties such as the population of Chemits, propagation, replication events over the total simulation time. An example code written in Mathematica 12 notebook is available to download from GitHub. We ran up to 8 simulations in parallel using Mathematica's parallel kernels. Once the simulation is completed, chemical states and PWM states at each time step were stored and dumped and Mathematica matrix object files (*.mx). A separate script also based on Mathematica was used to analyse the data and estimate the useful properties. In the next subsections, we will show results from simulations with variations in input parameters such as initial populations, size of the experimental space, frequency of random events.

### Variable Initial Population

We ran simulations with various numbers of initial Chemits {1, 10, 100, 1000} over a 100×100 experimental grids for 15000 steps with 500 random actuation events at each time step (Snapshots of simulation of 2D-CCA using a phenomenological mode not shown). With the different initial numbers of Chemits, we ran simulations 25 times for each case. From the simulation data, we estimated the number of Chemits by estimating the number of PWM₃ states at each time step.

Independent of the initial populations, we observe the average population in each case converge to ca. 58 Chemits at 15,000 simulation steps (population distribution of Chemits with different initial populations over time not shown). When the initial population of Chemits is low, e.g. initial populations are 1 and 10, we observe typical growth kinetics of the Chemits and eventually reaches the steady-state population which for the current set of parameters is ca. 58 Chemits. When the initial population is higher than the steady-state, especially in the case when the initial population is 1000, strong annihilation events occur fast due to the competition logic.

We also quantified propagation, replication and annihilation events throughout the simulation. To estimate these quantities, we first create a list of positions of all Chemits using PWM₃ states. At each time step, as a first step, we calculate the number of still states by finding the intersection between PWM₃ states at time t and t−1. The disappeared states between two consecutive steps occur due to propagation and annihilation events. The new appearing states between two consecutive steps occur due to propagation and replication events. For each of the new species appeared, we calculated the list of nearest neighbour and next-nearest neighbours.

Based on the positions of new states and neighbours from the old states, we estimate the propagation and replication events. We estimate the annihilation events by subtracting the difference between new and old states from the replication events. A Mathematica Notebook to analyse the simulation data is available on GitHub. With the single Chemit, replication and propagation kinetics is slow at the start of the simulation. Once the number of species has crossed a threshold value, the growth rate, propagation and replication events start accelerating and reach steady-state value near the end of the simulation.

### Variable Random Events

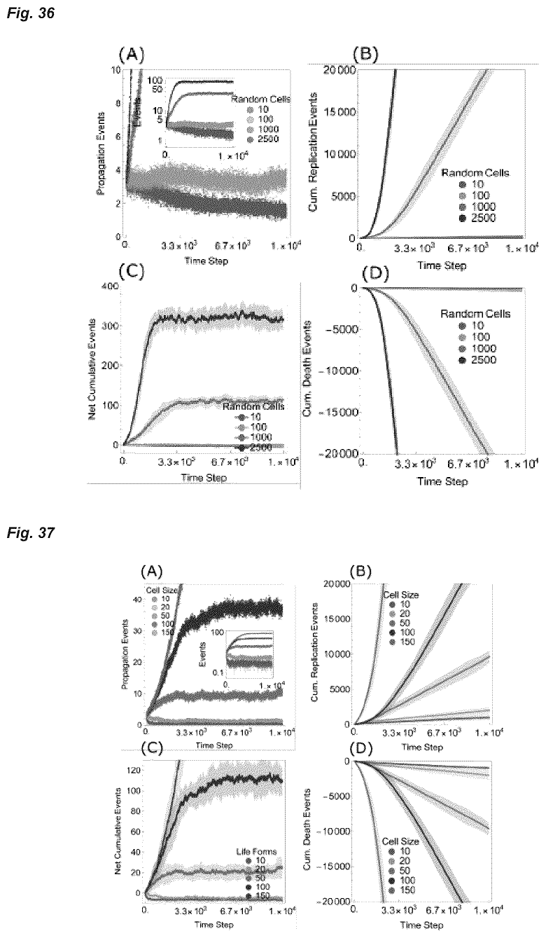

Starting at the same population of the Chemits (10), we ran stochastic simulations with the different number of active random cells at each time step on a 100×100 grid. The total simulation steps were 10000 for each case and the given set of conditions, each simulation was performed 25 times and averaged properties were estimated. As the total number of cells in the two-dimensional grid 10000 (100×100), we chose active random cells up to 2500. The number of active random cells is equivalent to the active number of PWM2 states at each time step. The number of active random cells represents the fluctuations emerging in the surrounding environment of the Chemits. The higher the number of fluctuations, the higher is the energy available in the surroundings. As described in the phenomenological model, the presence of random cell (with PWM2 state) increases the probability of the emergence of higher chemical state (CS), a significant increase in both propagation and net replication kinetics was observed (FIG. 36).

### Variable Total Cell Grid Size

We investigated the effect of overall space available for Chemits to proliferate by changing the total cell grid size, 10×10, 20×20, 50×50, 100×100, 150×150. The total simulation steps were 10000 for each case and the given set of conditions, each simulation was performed 25 times and averaged properties were estimated. The initial population of Chemits in each case was kept the same (10 Chemits). The total random events at each time (defined by PWM2) states were chosen based on a fixed ratio of the total number of available cells (1/10). As shown previously in the case of a different number of initial populations, that the Chemits show the tendency to achieve a steady-state population, the number of Chemits at the steady-state is controlled by the total amount of available space.

The available space can be seen as an available resource for the Chemits to proliferate or as a parameter which constraints the population of the Chemits and sets an upper bound (for the given set of other conditions such as random fluctuations). The variations in the propagation and replication dynamics at a different number of cell grid size is shown in FIG. 37.

### Exploration of Probabilistic Events Phase Space

To extend our investigation on the dynamics of emergent Chemits using hybrid electronic-chemical logic, we explored the phase space of the parameter Q2 and Q4 which describes the strength of the PWM states defining the random fluctuations (PWM2) and Chemit interaction cells (PWM4). The effect of PWM4 is always stronger than PWM₂, and Chemit interaction cells stirrer rotates always stronger than the random fluctuation cell stirrers, so we only considered the cases when Q4>Q2. We chose discrete values of Q2 and Q4 as {0.1,0.25,0.3,0.5,0.66,0.75,0.87,1}. We ran simulations for 10,000 steps for all the combinations of Q2 and Q4 such that Q4>Q2 was met. At each step, the number of randomly fluctuating cells was set to 500. For each set of parameters, 25 simulations were run to estimate the averaged behaviour (results now shown). Both Q2 and Q4 parameters are critical and enhance the probability of propagation and replication events, which is indicated by the monotonic increase in the steady-state populations.

### BZ Computation

### Implementation of Hybrid Electronic-Chemical Logic

Similar to those in 2D-CCA, we implemented a Finite State Machine logic in silico which reads in chemical states and updates the PWM states which are applied to the cell and interfacial stirrers. The evolution of chemical states is dominated by physicochemical principles which leads to the emergence of short-term memory or hysteresis effects in the system. In the current strategies to implement hybrid electronic-chemical computation logic, the combinatorial optimization problem is initialized by formulating an Ising Hamiltonian as a function of spin variables. Depending on the number of pairwise couplings, the computational problem was mapped to the two-dimensional experimental setup such that all the pairwise coupling variables in the Hamiltonian can exist as nearest neighbours. In the two-dimensional geometry, if all the couplings cannot be described by nearest neighbours, multiple instantiations of the same variables were created to map all the necessary pairs. These additional cells were called as auxiliary cells, and they flow the same logic as the primary cell associated with the given variable. Either the chemical states or PWM states were interpreted as Ising equivalent spins. At the start, all the spins are initialized randomly making sure that the auxiliary spins have the same states as the primary spins using. The emerging chemical states were read into the hybrid state machine, which updates the new PWM states. This information processing loop between digital and chemical domains was applied until the minimal energy configuration was achieved. Once the minimal energy configuration was achieved, the state of spin variables was interpreted as the solution to the combinatorial optimization problem.

### Electronic-Chemical State Machine Type 1

In this hybrid electronic-chemical state machine, we used chemical states of the cells as the representation of Ising equivalent spins, where high chemical state CS1 corresponds to +1 state and low chemical state CS0 corresponds to −1. The Hamiltonian function was created in-silico and the system was initialized to random spins (chemical states). The basic pseudocode is defined as,
1. Initialize PWM states to randomly assign the Chemical States to all the cell associated with Hamiltonian variables.
2. Assign the lowest energy to be infinity.
3. While True:
a. Randomly flip the PWM state of a cell and observe the emergence of all the new Chemical States.
b. Calculate current Energy based on the Chemical States.
c. If current Energy≤lowest energy:
lowest energy=current Energy
d. Accept or Reject the flipping based on the estimated energy difference and update the lowest energy. The probability of accepting the change is:

$${p}_{accept} = Max ( {e}^{\frac{- Δ E}{k}} , 1 )$$

where ΔE is the energy difference after flipping the spin and we set k=5.
e. If the lowest energy cannot be further reduced (termination):
Save current spins and interpret them as a solution.
Break

This is a simplistic approach, where most of the information processing occurs in the digital domain, however the information loops through the chemical and digital domain. There is also inherent hysteresis in the system from the physicochemical processes which also influences the chemical states and prevents the system to get stuck in local minima. This step is crucial towards implementing more advanced logic and computational algorithms. Using this approach, we solved three different combinatorial optimization problems.

### Electronic-Chemical State Machine Type 2

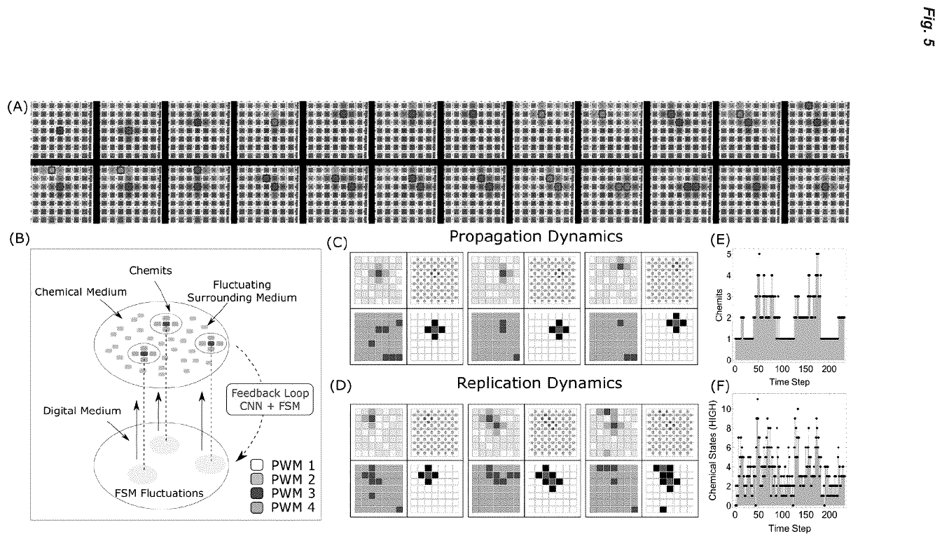

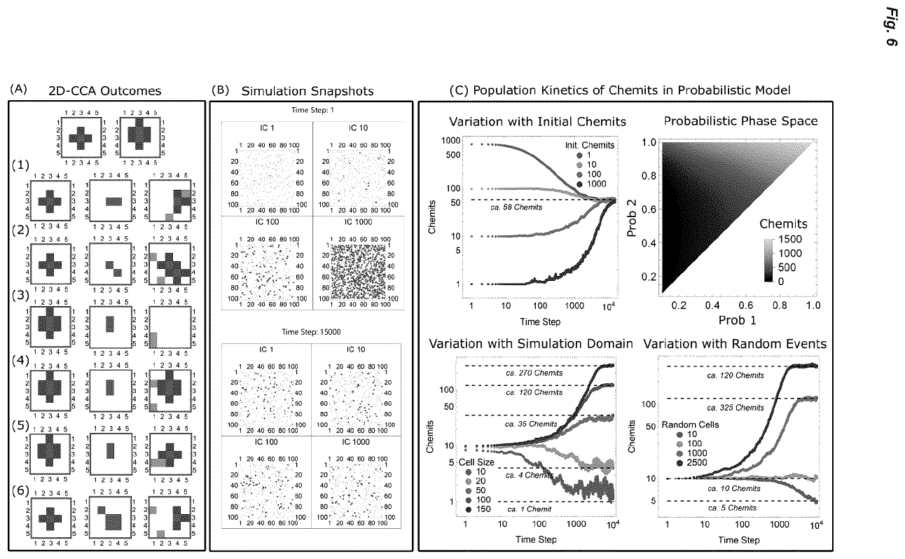

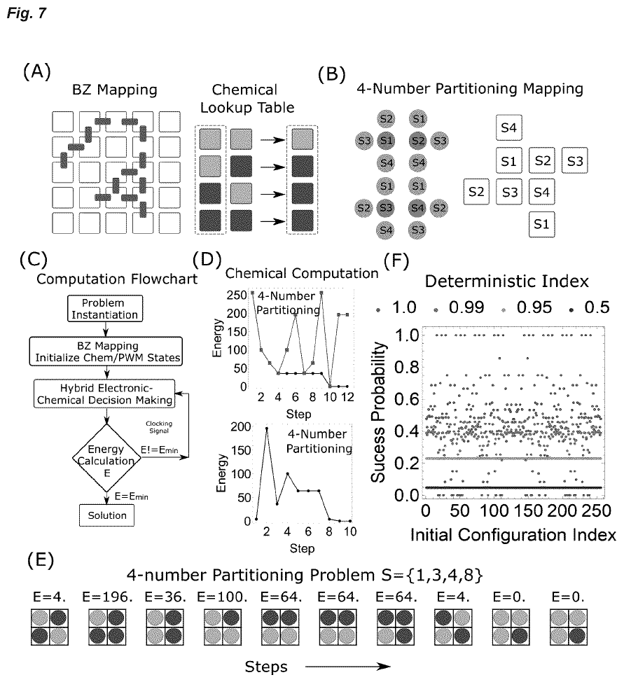

In this hybrid electronic-chemical state machine, we used PWM states of the cell stirrers to represent Ising spin variables. The Ising spin variables based on all the pairwise coupling terms were mapped into the experimental domain using primary and auxiliary cells. Similar to the previous case, the Hamiltonian function was created in-silico and the system was initialized to random spins (PWM states). The hydrodynamic interactions between the neighbouring cells were introduced as a part of the hybrid logic. The quadratic formulation of the Hamiltonian allows writing the energy terms specific to a given cell (both primary and auxiliary) as the summation of interactions between the given cell and neighbouring cells. Similar to the first hybrid state machine, we flipped the PWM state randomly and introduce the effect of local interactions between the cells when we estimate the energy change for the pairwise interactions. To introduce chemical decision making, a comparison was made between the emerging chemical states and the lookup table which describes the ideal emergence of chemical states in the absence of hysteresis and noise effects (see FIG. 7). Even though the phenomenological state machine is probabilistic, we can still estimate the most probable results from the local interactions logic. The overall energy change was calculated pair-wise and while calculating for a specific pair, if the observation of new chemical state is inconsistent with the lookup table, we change the sign of the energy change of this pair. The overall energy changed is accumulated pair-wisely and, similar to that in the first strategy, a decision is made to accept or reject the change of the PWM state using a Finite State Machine. The basic pseudocode is defined as,

```
1. Initialize PWM states randomly for all Ising spin variables associated
with the problem Hamiltonian.
2. While True:
a. Randomly flip the PWM state of the primary cell and create auxiliary
cells related to all pairwise interactions.
b. Set energy_benefit = 0
c. For all possible pair interaction including primary cell:
  Calculate pairwise energy change after flipping the PWM state
  Create the auxiliary cell and activate interfacial stirrers for interactions
  If the new Chemical States consistent with lookup table:
    d_energy = pairwise energy change
  Else:
    d_energy = − pairwise energy change
  Energy benefit = Energy benefit + d_energy
f. Accept the flipping if Energy benefit < = 0.
g. If the configuration energy reached minima (termination):
  Save current spins and interpret them as a solution.
  Break
```

In this approach, we introduced a hybrid electronic-chemical logic where information processing loops between chemical and electronic domains. Similar to the previous logic, calculation of energy occurs in the electronic domain, however, energy benefit for each step is based on the chemical decision making which occurs due to strong hysteresis, non-linear couplings and noise which are inherent in the system. Using this approach, we solved three different combinatorial optimization problems, whose associated Hamiltonians have been described in the previous subsection.

### Qualification of Chemical Computation

In this section, we describe the role of chemistry in the computational logic as implemented in the proposed hybrid electronic-chemical computer. To demonstrate the computation in hybrid electronic-chemical architecture, we redefine the question as to the following,

In this document, we describe the role of chemistry in the computational logic as implemented in the proposed hybrid electronic-chemical computer. To demonstrate the computation in hybrid electronic-chemical architecture, we redefine the question as follows:
1. Do one-& two-dimensional Chemical Cellular Automata and solving combinatorial optimization problems using the computational architecture give the complete picture towards hybrid computation?
2. Is there a one-to-one mapping between the electronic and chemical domain, such that digital logic controls all the phenomena and information just loops between electronic and chemical domain without any additional benefit?
3. What does the mapping between input PWM states and emerging chemical states look like?
4. Is it possible to simulate the complete electronic-chemical computational model in-silico and if yes, what does the resource usage looks like?

In the following sections, we will describe our computational hybrid state machine in context to one- and two-dimensional Chemical Cellular Automata (CCA) as well as for chemical computation logic for combinatorial optimization problems. Based on our description, we aim to answer all four proposed questions. In the implemented hybrid computational logic, at each step the information loops between digital and chemical domains, where different parts of computation take place. To describe them clearly, as a first step we distinguish two different aspects of our hybrid computational architecture including information transfer and processing.

### 1. Information Transfer and Time Stepping

The information transfer from the electronic to the chemical domain and vice versa is based on time-stepping logic which is essential for the proposed computational architecture. As the hybrid logic uses state machines in both digital and chemical domains, dynamic communication between them is essential and is considered as a separate process from Information Processing. The two information communication processes between electronic and chemical domains are described below.
a. Electronic to Chemical: Information transfer form electronic domain to chemical domain occurs by mapping the output of digital Finite State Machine to the PWM levels of mechanical stirrers, which influences the emergence of chemical states.
b. Chemical to Electronic: Information transfer from the chemical state to the electronic state occurs through imaging and a recognition state machine based on a neural network (CNN) that converts analogue chemical states ( ) to the digital equivalent of chemical states (C_{S}).
c. Time Stepping: Time stepping is an essential feature to synchronize chemical and electronic logic and is defined by the chemical oscillation clock in the chemical domain.

### 2. Information Processing and Computational Operations

The information processing logic and the implemented algorithms utilise both electronic and chemical processes. These include the digital and chemical states as well as the various state machines which act on chemical and digital states. Here, we define all states and state machines,
1

$$\underline{{CS}_{i}^{t}}$$

—defines the analogue chemical state of the i^{th} cell at time step t which is equivalent to the chemical oscillation.
2.

$$\underline{{CS}_{i}^{t}}$$

—defines the digital chemical state of the i^{th} cell at time step t which is the outcome of the recognition state machine based on a neural network (CNN) applied on the analogue chemical state.
3. S^{t}ᵢ—defines the digital PWM state of the cell stirrer of the i^{th} cell at time t.
4.

$${I}_{i , j}^{t}$$

—defines the digital PWM state of the interfacial stirrer placed between the i^{th} and j^{th} cell at time t, where i and j are neighbouring cells.
5. T(CNN)—defines the state machine which acts on the analogue chemical state

$$( \underline{{CS}_{i}^{t}} )$$

and converts it into the digital chemical state (CS^{t}ᵢ).
6. D—defines the digital Finite State Machine which acts on CS^{t}ᵢ and updates cell stirrer states (S^{t}ᵢ) and the interfacial stirrer states (I^{t}_{i,j}).
7. P—defines the function which reads the PWM states and brings their effect to chemical analogue states in a physical world.
8. C—defines the chemical state machine which reads in the previous analogue chemical state (CS^{t}ᵢ) and analogue equivalent interaction of the stirrer using function P. It also includes the T (CNN) state machine and hence directly updates the digital chemical state CS^{t}ᵢ. It is important to note that even CNN occurs in the digital medium, we still consider it as a part of C as it links two different versions of the chemical state.
9. K—defines the hybrid state machine which includes all the state machines (T, C, P), such that it reads digital chemical state and outputs directly the new chemical state.

The complete representation of the hybrid electronic-chemical state machine is shown in, where information loops between the chemical and the digital domain. Based on the definition of various state variables and machines, we describe the electronic and chemical operations in details.

Electronic Computation comprises the digital Finite State Machine (D) which takes the digital chemical states of the cell and its neighbours as inputs and updates the new PWM states to be applied on the stirrers. Assuming only nearest neighbours in one-dimensional CCA, the new cell stirrer state and the two interfacial stirrers states can be defined as,

$$\{ {S}_{i}^{t} , {I}_{i , i - 1}^{t} , {I}_{i , i + 1}^{t} \} = D ( {CS}_{i}^{t} , {CS}_{i - 1}^{t} , {CS}_{i + 1}^{t} )$$

This general formulation of the state machine can be defined for any dimensions and higher-order neighbours as well. The digital state machine is deterministic and activates by the chemical clock signal.

Chemical Computation comprises a combination of two different state machines and, hence reads the digital PWM states and updates the new chemical states based on the physical phenomena which include temporal BZ oscillation chemistry coupled with hydrodynamic interactions from the stirrers.

It is important to note that the emergence of a new chemical state has both implicit and explicit dependence on the previous chemical state. By explicit, we mean the previous states determine the PWM level explicitly through P and this PWM level influences the emergence of CS, while the hysteresis effect from previous operations is implicit.

Furthermore, the emergence of a new digital chemical state is a complex function of the previous chemical state and multiple physical interactions which include neighbouring interactions and interaction of stirrers on the ongoing chemical oscillation. So, for one-dimensional CCA, we can write a new digital chemical state as,

$${CS}_{i}^{t + 1} = f ( {CS}_{i}^{t} , {S}_{i}^{t} , {S}_{i - 1}^{t} , {S}_{i + 1}^{t} , {I}_{i , i + 1}^{t} , {I}_{i , i - 1}^{t} )$$

where we assume there is no direct dependence on the chemical states of the neighbouring cells and is a function that takes into account all the physical effects related to the given variables. We start with simplifying the function by excluding the neighbouring interactions and considering the central cell chemical state and PWM state of the cell stirrers. The functional relationship between current and previous chemical state is defined by (which has both hybrid electronic and chemical interaction components)

$${CS}_{i}^{t + 1} = f ( {CS}_{i}^{t} , {S}_{i}^{t} )$$

Even we expect (x) to be a non-linear function, we split it into two different parts to distinguish chemical and electronic processing by assuming linear combinations of explicit (digital) and implicit (chemical) dependence of the previous chemical state with is the weighting factor.

$${CS}_{i}^{t + 1} = α ( \underline{{CS}_{i}^{t}} ) + ( 1 - α ) P ( {S}_{i}^{t} ) = α ( \underline{{CS}_{i}^{t}} ) + ( 1 - α ) P ( D ( {CS}_{i}^{t} ) )$$

It is important to understand the difference between functions which involves and even when both represent different aspects of the same chemical state. The two terms in the equation clearly show the separation of chemical and electronic processes in hybrid computation.

$$α ( \underline{{CS}_{i}^{t}} )$$

occurs in a chemical medium and relates to the Hysteresis effect and noise.

$$( 1 - α ) P ( {S}_{i}^{t} )$$

occurs in the chemical medium and is a physical process that includes hydrodynamic interaction between existing chemical oscillation that occurs over a single clocking step.

$${S}_{i}^{t} = D ( T ( {CS}_{i}^{t} ) )$$

occurs in the digital medium using a Finite State Machine (D).

Step 1 and 2 corresponds to chemical information processing and are probabilistic while Step 3 corresponds to digital information processing which is deterministic. In our experimental design, the parameter also tunable by careful selection of PWM levels. This flexibility in our computational architecture allows us to switch between deterministic and probabilistic domains for the efficient implementation of hybrid computation algorithms.

As a next step, we extend our formulation by introducing the effect of nearest neighbours. In the case of one-dimensional CCA, as discussed previously the functional relationship can be defined as

$${CS}_{i}^{t + 1} = f ( {CS}_{i}^{t} , {S}_{i}^{t} , {S}_{i - 1}^{t} , {S}_{i + 1}^{t} , {I}_{i , i + 1}^{t} , {I}_{i , i - 1}^{t} )$$

Similarly, we formulate f as a linear combination of different interactions which can be described as

$${CS}_{i}^{t + 1} = α ( \underline{{CS}_{i}^{t}} ) + β {P}_{C} ( {S}_{i}^{t} ) + ( 1 - α - β ) P ( {S}_{i}^{t} , {S}_{i - 1}^{t} , {S}_{i + 1}^{t} , {I}_{i , i - 1}^{t} , {I}_{i , i + 1}^{t} )$$

Here, we introduced two different types of transfer functions for different coupling interactions

$${P}_{C} ≡ {P}_{C} ( {S}_{i}^{t} )$$

describes the physical effect of the central cell stirrer on the chemical oscillation.

$${P}_{I} ≡ {P}_{I} ( {S}_{i}^{t} , {S}_{i - 1}^{t} , {S}_{i + 1}^{t} , {I}_{i , i - 1}^{t} , {I}_{i , i + 1}^{t} )$$

between neighbouring cells. This is complex phenomena due to the coupling vortices of three neighbouring cells via interfacial stirrers.

The various electronic operations which use the digital finite state machine D are defined as,

$${S}_{i}^{t} ≡ {S}_{i}^{t} ( {CS}_{i}^{t} , {CS}_{i + 1}^{t} , {CS}_{i - 1}^{t} )$$

$${I}_{i , i - 1}^{t} ≡ {I}_{i , i - 1}^{t} ( {CS}_{i}^{t} , {CS}_{i - 1}^{t} )$$

$${I}_{i , i + 1}^{t} ≡ {I}_{i , i + 1}^{t} ( {CS}_{i}^{t} , {CS}_{i + 1}^{t} )$$

$${S}_{i}^{t} , {I}_{i , i - 1}^{t} , {I}_{i , i + 1}^{t} = D ( {CS}_{i}^{t} , {CS}_{i + 1}^{t} , {CS}_{i - 1}^{t} )$$

In this formulation, the role of digital and chemical information processing can be separated and can be described by the following,

$$α ( \underline{{CS}_{i}^{t}} ) \qquad 1.$$

occurs in a chemical medium and relates to the Hysteresis effect and noise

$$2. β {P}_{C} ( {S}_{i}^{t} )$$

occurs in a chemical medium describes the physical effect of the central cell stirrer on the oscillations.

$$3. ( 1 - α - β ) {P}_{I} ( {S}_{i}^{t} , {S}_{i - 1}^{t} , {S}_{i + 1}^{t} , {I}_{i , i - 1}^{t} , {I}_{i , i + 1}^{t} )$$

occurs in the chemical medium describes the physical effects of hydrodynamic coupling between neighbouring cells on the existing chemical oscillations.

$$4. \{ {S}_{i}^{t} , {I}_{i , i - 1}^{t} , {I}_{i , i + 1}^{t} \}$$

occurs in the electronic domain controlled by the digital Finite State Machine which is defined as

$${S}_{i}^{t} , {I}_{i , i - 1}^{t} , {I}_{i , i + 1}^{t} = D ( {CS}_{i}^{t} , {CS}_{i + 1}^{t} , {CS}_{i - 1}^{t} )$$

### Time Stepping in Hybrid Electronic-Chemical Logic

Using the description of state machine and variables which distributes information processing in electronic and chemical domains, we now describe the complete hybrid state machine K, and formulate time-stepping logic where at each step the information loops through chemical and electronic domains. The hybrid state machine uses digital processing (D) and physical and chemical processing state machines (P,C). To distinguish the initial state from the generalized state at time step t, we describe an additional state machine C₀ which represents the initial condition. The hybrid chemical-digital state machine K acting on a chemical state is defined as

$${K}^{t} ( {CS}_{i} ) ≡ C ( P ( D ( {CS}_{i} , {CS}_{j} ) ) , \underline{{CS}_{i}} )$$

such that, we can describe time-stepping of the digital chemical state as,

$${CS}_{i}^{t + 1} = {K}^{t} ( {CS}_{i}^{t} , {CS}_{j} )$$

where CSᵢ^{t} represents the digital chemical state of the central cell and CSⱼ describes the combined digital chemical state of all the neighbouring cells. At the initial state, for any CCA, the chemical state and PWM state of the underlying stirrer has one-to-one mapping which is controlled by the initial chemical state machine C₀ without any surrounding hysteresis effects and noise effects from chemical fluctuations.

The initial condition is well-defined by digital chemical states or equivalent stirrer operations and at this point, chemical interactions start.

$$IC = {CS}_{i}^{0} = {C}_{0} ( {S}_{i}^{0} , {I}_{i , j}^{0} )$$

$$IC = ( {S}_{i}^{0} , {I}_{i , j}^{0} ) = D ( {CS}_{i}^{0} )$$

The initial condition is well-defined by digital chemical states or equivalent stirrer operations and at this point, chemical interactions start.

The temporal evolution is given by the interaction of the cell with its neighbouring cells together with hysteresis, hydrodynamic coupling and noise effects defined by chemical state machines and P with the given initial conditions,

### Step 1:

$${CS}_{i}^{1} = C ( P ( {S}_{i}^{0} , {I}_{i , j}^{0} ) , \underline{{CS}_{i}^{0}} ) = C ( IC )$$

$$( {S}_{i}^{1} , {I}_{i , j}^{1} ) = D ( {CS}_{i}^{1} , {CS}_{j}^{1} )$$

For the next step, to update the digital chemical state CSᵢ², we can write Step 2 as:

### Step 2:

$${CS}_{i}^{2} = C ( P ( {S}_{i}^{1} , {I}_{i , j}^{1} ) , \underline{{CS}_{i}^{1}} ) = C ( P ( D ( {CS}_{i}^{1} , {CS}_{j}^{1} ) ) , \underline{{CS}_{i}^{1}} ) = {K}^{1} ( C ( IC ) )$$

$$( {S}_{i}^{2} , {I}_{i , j}^{2} ) = D ( {CS}_{i}^{2} , {CS}_{j}^{2} )$$

where the hybrid state machine can be expanded as,

$${K}^{1} ( C ( IC ) ) = C ( P ( D ( C ( IC ) ) ) , C ( IC ) )$$

$${CS}_{i}^{3} = C ( ( {S}_{i}^{2} , {I}_{i , j}^{2} ) , \underline{{CS}_{i}^{2}} ) = C ( P ( D ( {CS}_{i}^{2} , {CS}_{j}^{2} ) ) , \underline{{CS}_{i}^{2}} ) = {K}^{2} ( {K}^{1} ( C ( IC ) ) ) \qquad Step 3$$

$$( {S}_{i}^{3} , {I}_{i , j}^{3} ) = D ( {CS}_{i}^{3} , {CS}_{j}^{3} )$$

$${CS}_{i}^{N} = C ( ( {S}_{i}^{N - 1} , {I}_{i , j}^{N - 1} ) , \underline{{CS}_{i}^{N - 1}} ) = C ( P ( D ( {CS}_{i}^{N - 1} , {CS}_{j}^{N - 1} ) ) , \underline{{CS}_{i}^{N - 1}} ) = {K}^{N - 1} {K}^{N - 2} ... {K}^{1} ( C ( IC ) ) \qquad Step N$$

$$( {S}_{i}^{N} , {I}_{i , j}^{N} ) = D ( {CS}_{i}^{N} , {CS}_{j}^{N} )$$

Hence, the general formulation for the digital chemical state at the t^{th} time step to a hybrid chemical-electronic state machine is defined as,

$${CS}_{i}^{t} = {K}^{t - 1} {K}^{t - 2} ... {K}^{1} ( C ( IC ) )$$

$${K}^{t} ( {CS}_{i} ) ≡ C ( P ( D ( {CS}_{i}^{t} , {CS}_{j}^{t} ) ) , \underline{{CS}_{i}^{t}} )$$

So, a hybrid chemical-electronic state machine K comprised of three different operations which occur in the digital and analogue domain defined by three state machines D, P and C. Based on this formulation of the hybrid chemical-electronic state machine, we can write our state machine in computation and “display screen” mode can be defined,

$${K}_{comp}^{t} ( {CS}_{i}^{t} ) ≡ C ( P ( D ( {CS}_{i}^{t} , {CS}_{j}^{t} ) ) , \underline{{CS}_{i}^{t}} )$$

$${K}_{disp}^{t} ( {CS}_{i}^{t} ) ≡ C ( P ( D ( {CS}_{i}^{t} , {CS}_{j}^{t} ) ) ) = H ( D ( {CS}_{i}^{t} , {CS}_{j}^{t} ) - p )$$

$$H ( x ) = \{ \begin{matrix}0 , & x < 0 \\ 1 , & x \geq 0\end{matrix}$$

where K^{t}_{comp} is the hybrid state machine in computation mode, K^{t}_{disp} is the hybrid state machine in a fully deterministic display screen mode, H(x) is a piecewise function and p is the threshold stirrer level at which the digital chemical state changes. In the display screen mode, there are no hysteresis, physical coupling and noise effects, so that there exists a one-to-one mapping in the digital PWM states and chemical states.

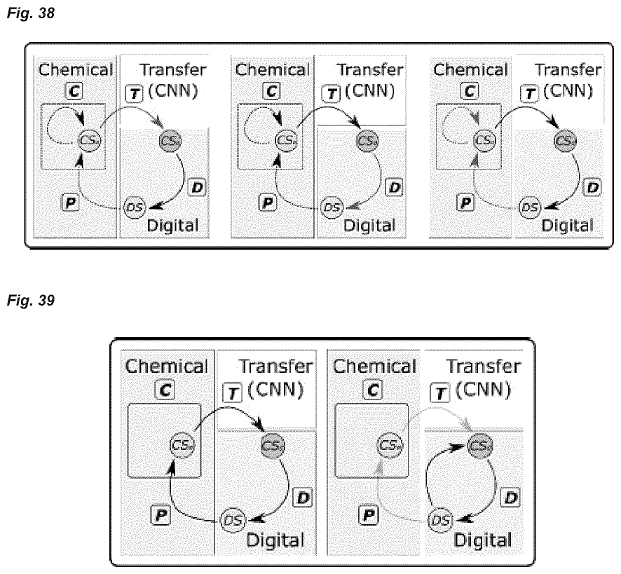

The pictorial representation of the hybrid chemical-electronic state machine in computation mode and in “display screen” mode is shown in FIGS. 38 and 39.

### Emergence of Probabilistic Behaviour in Hybrid Computational Architecture

In this section, we will describe the emergence of the probabilistic nature of computation in our proposed computational architecture. Given an initial state, we define response as the effect on the initial state due to the added stimulus of control input. Starting from the digital PWM states which are the output of the digital state machine, the response of the PWM value on the stirrer (motor) can be assumed to be linear. If we define emerging states based on only the response of the stirrer, we can distinct two output states and hence, well-defined deterministic output. Chemical response to stirrer input becomes non-linear, with a region where the stirrer response on emerging chemical states is strong. We have demonstrated similar behaviour experimentally, where the peak oscillation amplitude rises strongly with the change in PWM values by creating a step function to increase the PWM values from 20 to 80.

Due to short term memory in the chemical system (forced-damped oscillator), the system shows strong hysteresis behaviour which leads to a complex interaction between the previous oscillatory state and non-linear chemical response from the stirrer. These emergent analogue chemical states when classified with CNN leads to probabilistic chemical states. However, during the actual computation, the interaction is not limited to the previous chemical oscillatory states and stirrer response, but also with the interactions of nearest neighbours and response from the multiple interacting cells and interfacial stirrers. The coupled oscillatory chemical states with high-dimensional nearest neighbours, hysteresis and stirrer interactions expand the probabilistic space, which when gets classified by CNN leads to probabilistic outcomes.

Based on the defined hybrid chemical-electronic state machine above, we further extend our formulation towards computation in one- and two-dimensional Chemical Cellular Automata (CCA) and identifying the role of chemistry in the computational architecture.

### Example 1: One-Dimensional Chemical Cellular Automata

In the one-dimensional CCA implementation, consider a 1D-CCA rule defined by DS-DI where the finite state machine DS updates the central cell stirrer and DI updates the interfacial cell stirrers based on readout of digital chemical states. In this case, the transfer function state machine takes the output from two digital finite state machines (DS and DI) and transfers both cell and interfacial stirrer operations into the chemical analogue domain. Hence, the generalized 1D-CCA hybrid chemical-electronic state machine is defined as,

K^{t}(CSᵢ^{t})≡C(P({DS(CSᵢ^{t},CSⱼ^{t}),DI(CSᵢ^{t},CSⱼ^{t})}),CSᵢ^{t})

The various implemented 1D CCA rules utilize the generalized hybrid state machine, where digital state machines DS and DI) were defined in a deterministic way and chemical/physical state machines (D,C) was implemented using a phenomenological probabilistic model. The emergence of novel patterns occurs due to state space expansion due to chemical information processing defined by (D,C)

Similarly, the implemented elementary CA rule state machine (“display screen mode”) can be defined from the generalized state machine as,

$${K}_{el}^{t} ( {CS}_{i}^{t} ) ≡ C ( P ( \{ DS ( {CS}_{i}^{t} , {CS}_{j}^{t} ) \} ) ) = H ( DS ( {CS}_{i}^{t} , {CS}_{j}^{t} ) - p )$$

which is equivalent to the 1D-CCA rule DS-0, where there is no interaction between the neighbouring cells as interfacial stirrers are off and direct one-to-one mapping on the central cell for the given rule defined by DS.

### Example 2: Two-Dimensional Chemical Cellular Automata

In the 2D CCA, the digital finite state machine that leads to propagation, replication, competition and multiple events of chemical entities (Chemits) is defined by D_{C}(CS_{i,j}^{t},CS_{m,n}^{t}), which reads the local digital chemical states between the nearest and next-nearest neighbours where CS_{i,j}^{t} represents the central cell and CS_{m,n}^{t} represents all the neighbours collectively.

$${K}_{C}^{t} ( C {S}_{i , j}^{t} , {CS}_{m , n}^{t} ) \underline{=} C ( P ( {D}_{C} ( C {S}_{i , j}^{t} , {CS}_{m , n}^{t} ) ) , ( \underline{{CS}_{i , j}^{t} ,} \underline{{CS}_{m , n}^{t}} ) )$$

The finite state machine

$${D}_{C} ( C {S}_{i , j}^{t} , {CS}_{m , n}^{t} )$$

for Chemits comprises two different parts which output four different PWM states of cell stirrers and two for interfacial stirrers,

$$1. {D}_{1} ( C {S}_{i , j}^{t} , {CS}_{i , j}^{t} ) \to \{ {1}_{i , j}^{t} , {3}_{i , j}^{t} , {4}_{i , j}^{t} \}$$

with 1, 3 and 4 are PWM levels corresponding to inactive, cell core and interaction stirrers.

$$2. {D}_{2} ( C {S}_{i , j}^{t} ) \to \{ {1}_{i , j}^{t} , {2}_{i , j}^{t} \}$$

with 1 and 2 are PWM levels corresponding to inactive and random fluctuations stirrer to create surrounding weak oscillations.

The implementation of Chemical Entities (Chemits) in both experiments and simulations demonstrated emergent behaviour in the hybrid chemical-electronic state machine, where the Chemits interact with the fluctuating medium.

### Probabilistic Computational Operators Using Chemits as a Potential Route Towards Computation

We extend our formulation of the hybrid state machine for Chemits towards programmable interactions which could be utilized for probabilistic computational operations. The Chemits demonstrates four different events which can be interpreted or used to describe computational operations.

### 1. Propagation as Information Transfer or Connection Wire

Chemits propagation occurs due to its interactions with the surrounding fluctuating chemical field. Due to the probabilistic nature of the propagation dynamics, the hybrid state machine can be programmed in deterministic and probabilistic modes by controlling the state machines D1 and D2 which is equivalent to selecting the PWM values of surrounding stirrers in a biased and unbiased way. Both (D1,D2) can be programmed to make Chemits perform random walk to directional motion. So, the hybrid state machine for the propagation of Chemits is defined as,

$${K}_{T}^{t} ( C {S}_{i , j}^{t} , {CS}_{m , n}^{t} ) ≡ C ( P ( {D}_{1} ( {CS}_{i , j}^{t} , {CS}_{m , n}^{t} ) , {D}_{2} ( C {S}_{i , j}^{t} , {CS}_{m , n}^{t} ) ) , ( \underline{{CS}_{i , j}^{t}} , \underline{{CS}_{m , n}^{t}} ) )$$

where,

$${CS}_{i , j}^{t} and {CS}_{m , n}^{t}$$

defines the digital chemical state of the central cell and all nearest neighbours collectively. It is important to note that the state machine

$${K}_{T}^{t}$$

acts on digital chemical states and updates to new chemical states, however, we define our Chemit based on a combination of digital and chemical state. We represent this combined representation of the Chemit as

$${Q}_{i , j}^{t} .$$

So, instead or denning our state machines on digital chemical states

$$( {K}_{T}^{t} )$$

we define a propagation operator [symbol] _{T}^{t} which directly acts on

$${Q}_{i , j}^{t}$$

and updates to a new state.

$${𝒫}_{T}^{t} ( {Q}_{i , j}^{t} ) ≡ {𝒫}_{T}^{t} ( {S}_{i , j}^{t} , {S}_{m , n}^{t} , {K}_{T}^{t} ( {CS}_{i , j}^{t} , {CS}_{m , n}^{t} ) )$$

We utilize the propagation operator

$${𝒫}_{T}^{t}$$

to propagate the Chemit form the position (i, j) to its nearest neighbours (m, n), which corresponds to {(i+1,j), i=1,j),(i,j−1), (i,j+1)}. So, in the combined representation, the time stepping of Chemit is given by,

$${Q}_{m , n}^{t + 1} = {𝒫}_{T}^{t} ( {Q}_{i , j}^{t} )$$

Using this propagation operator for a random walk with and without drift, we can reach a position (p, q) from the initial position (i, j) by applying the propagation operators iteratively.

$${Q}_{p , q}^{t} = {𝒫}_{T}^{t} {𝒫}_{T}^{t - 1} ... {𝒫}_{T}^{1} {𝒫}_{T}^{0} ( {Q}_{i , j}^{0} ) = {𝒫}_{T}^{t} ( {Q}_{i , j}^{0} )$$

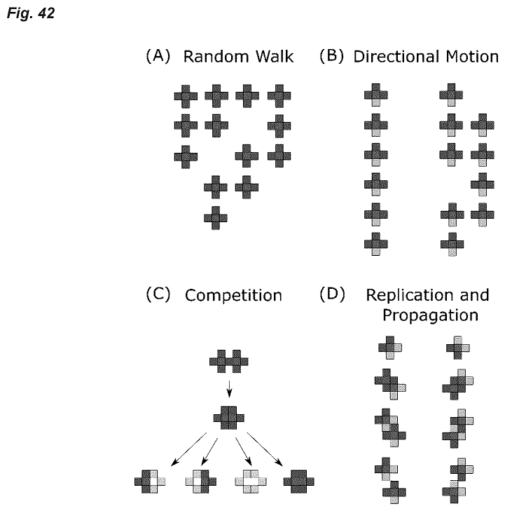

FIG. 42(A,B) shows a pictorial representation of a random walk and directional motion performed by a Chemit which can be controlled by selecting different PWM levels of interacting cells (PWM State 4), which can change the probability of propagation in the specific direction.

### Replication as Information Copy Process

Similar to the propagation event, Chemits replicates also due to its interactions with the surroundings. The replicating state machine can also be programmed in purely deterministic and probabilistic modes by controlling the state machines D₁ and D₂. The hybrid state machine for a replication event K^{t}ᵣₑₚ at the position (i,j) with digital chemical state CS^{t}_{i,j} and CS^{t}_{m,n}, corresponds to central cell and next-nearest neighbours

$$\{ ( i + 1 , j + 1 ) , ( i - 1 , j + 1 ) , ( i + 1 , j - 1 ) , ( i - 1 , j - 1 ) \} .$$

$${K}_{Rep}^{t} ( {CS}_{i , j}^{t} , {CS}_{m , n}^{t} ) ≡ C ( P ( {D}_{1} ( {CS}_{i , j}^{t} , {CS}_{m , n}^{t} ) , {D}_{2} ( {CS}_{i , j}^{t} , {CS}_{m , n}^{t} ) ) , ( \underline{{CS}_{i , j}^{t}} , \underline{{CS}_{m , n}^{t}} ) )$$

We can define a replication operator

$${𝒫}_{R}^{t}$$

which acts on Chemit defined by

$${Q}_{i , j}^{t}$$

at position (i,j) which equivalently can be used for bit copying.

$${𝒫}_{R}^{t} ( {Q}_{i , j}^{t} ) ≡ {𝒫}_{R}^{t} ( {S}_{i , j}^{t} , {S}_{m , n}^{t} , {K}_{Rep}^{t} ( {CS}_{i , j}^{t} , {CS}_{m , n}^{t} ) )$$

$${Q}_{m , n}^{t + 1} = {𝒫}_{R}^{t} ( {Q}_{i , j}^{t} )$$

Using this we can define replication-propagation operations, which copy to the chemical state and propagate to create complex interacting circuits as the example defined below. Copying the state from a position (i,j) and reach position (m,n) in k steps.

$${Q}_{m , n}^{k} = {𝒫}_{T}^{k} {𝒫}_{T}^{k - 1} … {𝒫}_{T}^{1} ( {𝒫}_{R}^{0} ( {Q}_{i , j}^{0} ) )$$

FIG. 42(D) shows replication and propagation events from a single Chemit, where the probability of replication events in a specific direction can be controlled by selecting different PWM levels of the interacting cells.

### Competition as Probabilistic Selection

The hybrid state machine for the competition event can be defined as

$${K}_{Comp}^{t}$$

wherein reads in two the high chemical states of two nearest neighbours

$$( {CS}_{i , j}^{t} , {CS}_{m , n}^{t} )$$

$${K}_{Comp}^{t} ( {CS}_{i , j}^{t} , {CS}_{m , n}^{t} ) ≡ C ( P ( {D}_{1} ( {CS}_{i , j}^{t} , {CS}_{m , n}^{t} ) , {D}_{2} ( {CS}_{i , j}^{t} , {CS}_{m , n}^{t} ) ) \underline{( {CS}_{i , j}^{t}} , \underline{{CS}_{m , n}^{t}} ) )$$

Using this hybrid state machine, various possible outcomes are possible which includes the survival of one, none or both Chemits. The selection probabilities for the outcomes can be implemented in any probabilistic machine and for simplicity, we implement it via the random number generator from a computer. We can define the competition operator P^{t}_{C} which acts on two species where outputs the news states of two Chemits based on the defined probabilities, where Q^{t}_{i,j}, represents the Chemit at position (i,j).

$${𝒫}_{C}^{t} ( {Q}_{i , j}^{t} ) ≡ {𝒫}_{C}^{t} ( {S}_{i , j}^{t} , {S}_{m , n}^{t} , {K}_{Comp}^{t} ( {CS}_{i , j}^{t} , {CS}_{m , n}^{t} ) ) ( {Q}_{i , j}^{t} , {Q}_{m , n}^{t} ) = {𝒫}_{C} ( {Q}_{i , j}^{t} , {Q}_{m , n}^{t} )$$

Using the combination of propagation and competition operators, we can define probabilistic combinational logic which could be utilized for computation. Assuming two Chemits' initial positions were given by (i,j), (m,n), by utilizing the propagation operators which propagate them to new positions (p,q), (r,s) which are the nearest neighbours. Once the Chemits reach the nearest neighbouring sites, they can interact with competition operators with various probabilistic outcomes.

The translation of Chemits are given by propagation operators,

$${Q}_{p , q}^{t} = {𝒫}_{T}^{t} {𝒫}_{T}^{t - 1} … {𝒫}_{T}^{1} {𝒫}_{T}^{0} ( {Q}_{i , j}^{0} )$$

$${Q}_{r , s}^{t} = {𝒫}_{T}^{t} {𝒫}_{T}^{t - 1} … {𝒫}_{T}^{1} {𝒫}_{T}^{0} ( {Q}_{m , n}^{0} )$$

The interaction of Chemits are given by the competition operators,

$$( {Q}_{p , q}^{t + 1} , {Q}_{r , s}^{t + 1} ) = {𝒫}_{C} ( {Q}_{p , q}^{t} , {Q}_{r , s}^{t} )$$

FIG. 42(C) gives a pictorial representation of the competition event of two Chemits with various probable outcomes due to hybrid state machine logic.

### Multiple Events

In the occurrence of multiple events where multiple high digital chemical states occur at neighbouring and nearest neighbouring sites, an operator can be defined to selected randomly between replication and propagation events. The hybrid state machine corresponding to the multiple events is given by with R as the random selection operator among all the nearest and next-nearest neighbours.

$${K}_{Mul}^{t} ( {CS}_{i , j}^{t} , {CS}_{m , n}^{t} ) ≡ ℛ ( {K}_{Pr}^{t} ( {CS}_{i , j}^{t} , {CS}_{m , n}^{t} ) , {K}_{Rep}^{t} ( {CS}_{i , j}^{t} , {CS}_{m , n}^{t} ) )$$

Based on the hybrid state machine for multiple events, a random selection operator can also be used for selecting between propagation and replication operators.

$$ℛ ( {Q}_{i , j}^{t} ) ≡ ℛ ( {S}_{i , j}^{t} , {S}_{m , n}^{t} , {K}_{Comp}^{t} ( {CS}_{i , j}^{t} , {CS}_{m , n}^{t} ) ) {Q}_{m , n}^{t} = ℛ ( {𝒫}_{R} ( {Q}_{i , j}^{t} ) , {𝒫}_{T} ( {Q}_{i , j}^{t} ) )$$

Based on various events in which Chemits interacts with purely deterministic and probabilistic outcomes, it is possible to create complex networks using a population of Chemits to develop higher-order computational logic. Any higher-order computational logic, such as a probabilistic logic/circuit could be defined by a set of replication (bit copy), propagation (connection) and competition (probabilistic outcomes) operators. These operators internally use chemical and digital state machines, where chemical state machines once initialized drives deterministic electronic operations based on probabilistic outcomes. The digital control using the PWM levels of stirrers allows the hybrid state machine to span the complete output phase space between deterministic and probabilistic outcomes leading to massive state space expansion.

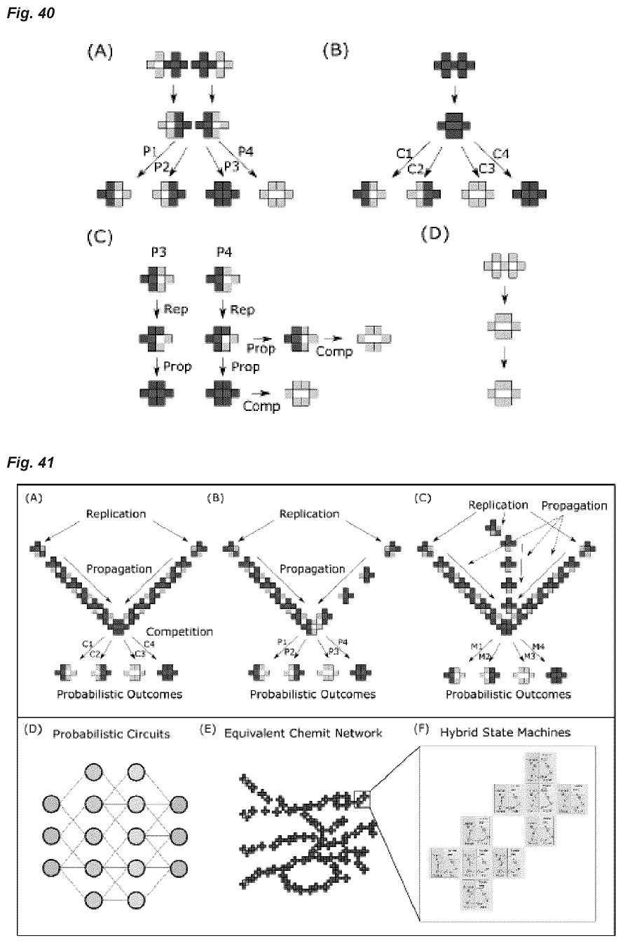

As an example, FIG. 40(A, B) shows the pictorial representation of the interacting Chemits using various operators leading to all possible outcomes based on replication, propagation and competition events.

As an example, FIG. 41 shows a pictorial representation of interacting Chemits with are emerging from static resources as an outcome of replication (bit copy) operator. The interaction between Chemits occurs via connecting path using propagation (connection) operator. At the interaction, depending on the local state various outcomes occurs based on the competition (probabilistic selection) operator (FIG. 41 A), all operators (FIG. 41 B) as shown before (FIG. 41 A and multiple events operators (FIG. 41 C). The outcome probabilities for all four outcomes are different for each case. As a potential example, we can envisage an equivalence of probabilistic logic/circuit defined by the equivalent Chemit network (see FIG. 41 D-F), where each point in the Chemit network is governing by local hybrid electronic-chemical state logic.

### Chemical Computation Towards Combinatorial Optimization Problems

In the previous section, we defined a hybrid state machine where both chemical and electronic state machines take part in the probabilistic computation. The computation in the two-dimensional Chemical Cellular Automata (CCA) shared between the chemical and digital domain, where the emergence of the chemical state drives the digital state machine. In this section, we extend the qualification of chemical computation by demonstrating the role of chemistry in solving combinatorial optimization problems.

Chemistry increases the number of the trajectories to connect different configurations in an Ising system in BZ computation, and we use a discrete-time Markov chain to understand and analyze it. To emphasize the role of chemistry, we started to introduce a greedy search algorithm and later combined it with chemical states which form our chemical computation strategy.

For a system with N spins, the total number of the configurations C is 2^{N}, where a configuration C is defined as a set of specific spins values. The transition between configurations forms the key to our optimization. If in two configurations, there is only 1 spin with different values and the rest of the spins are the same, they are neighbouring configurations. In a greedy algorithm, only the transition to neighbouring configurations with lower energy is allowed. Although it helps with fast convergence, this algorithm can be easily trapped in a local minimum and starting with some configurations, it will never reach the global minimum due to the limited number of configuration connections.

Chemistry is probabilistic in the sense even though we define the current PWM level, the emerged CS is not purely dependent on the current PWM values but a probabilistic distribution. Although this distribution depends on the accumulation of all the previous operations, we can control the PWM values so that this distribution is not completely random but quite consistent with our look-up table with a probability p_{chem}±Δp, where Δp is a small value counting the effects from previous states. We use a discrete-time Markov chain to analyze the optimization process given Δp is small.

First, the set of configurations are defined as C={c₁, c₂, . . . , c_{2N}} with that the individual configuration is a representation of the spins. Probability distribution in this configuration set is further defined with the initial condition P₀={p_{1,0}, p_{2,0} . . . , p_{2N,0}}. The probability distribution at time step tis represented by Pₜ={p_{1,t}, p_{2,t}, . . . p_{2N,t}} with Pₜ=P₀T^{t}, where is the transition matrix with a size of 2×2 and the summation within a row to be 1.

The (i^{th}, j^{th}) element in the transition matrix described the probability of configuration transition from cᵢ to cⱼ. It is important to note that only the transition between neighbouring configurations is allowed. Now the problem remains to compare the energy of two neighbouring configurations and which defines the (i^{th}, j^{th}) element in T. If the energy cⱼ is smaller than cᵢ, we change the configuration from cᵢ to cⱼ, else the configuration is still cᵢ. will be converged given t is large enough.

We only need to consider the transition of neighbouring configurations and those that are non-neighbouring are 0. Let us assume we flip the spin sₕ₁ to sₕ₂. Then the energy difference can be calculated as:

$$Δ E = ( {s}_{h 2} - {s}_{h 1} ) {\sum }_{i \neq h} {H}_{obs , ( h , i )} {K}_{h , i} {s}_{i}$$

Note the summation goes through all the 2^{N} spins except for spin h and K_{h,i} is the coefficients of the spin interactions. H_{obs(h,i)} is from the comparison between observation and the look-up table, which is the crucial step for us to introduce probabilistic effects from chemistry. If they are consistent, H_{obs(h,i)}=+1 else H_{obs(h,i)}=−1.

Since ΔE is calculated from individual interaction terms, the chance of H_{obs(h,i)} being 1 is p_{chem} for one term, which makes the overall distribution of ΔE being polynomial. If ΔE>0, this transition is rejected and ΔE≤0 is accepted, which contributes to the probability in the diagonal (no change) and (i^{th}, j^{th}) element in the transition matrix respectively.

If p_{chem} is 1, our algorithm reduces to the greedy search purely in the digital domain. If p_{chem} is 0.5, regardless of the spins, the probability of Δ≤0 is 0.5 where the system loses the tendency to minimize energy and become ergodic. By programming the chemical computer, we tune the value of p_{chem} and introduce the ergodicity from chemistry while enabling its tendency to minimize energy.

### Implementation Via Chemical and Digital State Machines

1. A set of spins S={S₁, S₂, . . . , S_{N}} is randomized and stored in the digital computer.
2. The HIGH or LOW PWM values in the corresponding cells are applied according to the spin state. (The digital finite state machine)
3. One spin sₕ is randomly flipped from sₕ₁ to sₕ₂ and the pair-wise energy change was calculated in the digital computer. (Which is (Sₕ₂−Sₕ₁)K_{h,i}Sᵢ, or every i spins) (The digital finite state machine)
4. Update the PWM values to the latest spin values, and the corresponding H_{obs(h,i)} was calculated from the chemical finite state machine. If the observation is consistent with the look-up table, H_{obs(h,i)}=+1 else H_{obs(h,i)}=−1. (The chemical finite state machine)
5. The overall energy change is calculated via Δ=(Sₕ₂−Sₕ₁)Σ_{i≠h}, H_{obs(h,i)}K_{h,i}Sᵢ and if ΔE<0, the change of the spin is accepted otherwise rejected.

### EXAMPLE: NUMBER PARTITIONING PROBLEM

Consider a number set S={1, 3, 4, 9, 3, 5, 3, 6} which needs to be partitioned into two disjoint subsets (S₁, S₂) such as the sum of all the elements of 1 and 2 is equal. We compare the performance of a purely digital and hybrid strategy. We started with setting p_{chem}=1, which creates a greedy algorithm. In this case, some initial configurations are already trapped in a local minimum, or they can lead to a local minimum, which means the probability of reaching the global minimum (thus solve the problem) is not possible. By slightly reducing p_{chem} to 0.99, the ergodicity of the system is enabled and all the configurations can reach the global minimum, by sacrificing the performance of some configurations.

Here we drew the probability of reaching global minimum starting with different configurations under different p_{chem}. With decreased p_{chem}, this distribution is shrunk which means the preference of selecting a “good” configuration is decreased after introducing the ergodicity.

### COMMENTS

In this document, we described the functionality of the proposed hybrid computation architecture and using the state-machine algebra, we demonstrated the role of electronic and chemical logic in constructing algorithms using the hybrid architecture. We posed questions at the start of the document which could be used to qualify our computational architecture towards the probabilistic chemical computer, which were answered in different aspects in various subsections. To answer these questions, we defined state variables and machines to describe information flow between electronic and chemical domains at each step in the hybrid computation with example in one- and two-dimensional CCA and combinatorial optimization problems. The roles of electronics and chemistry were shown by separating digital processing (deterministic finite state machine) from physical and chemical processes (probabilistic state machine).

Hence, demonstrating that one-to-one mapping is a subset of probabilistic mapping between electronic and chemical states. Using the one- and two-dimensional CCA, we demonstrated that the computational architecture could work in both deterministic and probabilistic modes, which increases the state space configurational paths through which the system can evolve. Using two-dimensional CCA, we proposed information processing steps (copy, connect, probabilistic logic) based on interactions of Chemits (replication, propagation, competition) with the possibility of mimicking a complex probabilistic circuit. We extended further by demonstrating a hybrid-computation algorithm to solve combinatorial optimization problem mapped to the Ising lattice ground state problem. We demonstrated the power of programmable probabilistic hybrid computation logic over deterministic algorithm by simplifying it to a Markovian process.

The description of the probabilistic algorithm as the Markov process was considered due to the simplicity of the model to describe the role of chemical and physical phenomena. As the problem size scales, hybrid logic prevents the system from getting stuck into local minima. For a very large system, we expect a large number of iterations with various initial configurations are required to find the global minima. Instead, using hybrid computation logic, a single run is enough to find the minima as it utilizes the probabilistic logic and has the capacity to explore alternate paths towards convergence with less tendency of getting stuck to local minima.

We have demonstrated that our computational architecture is probabilistic in nature where the information processing distributes between electronic and chemical domains and the implementation of simple algorithms towards useful computation. The computational architectural design is quite generic and there is a large scope of implementing more efficient algorithms over a diverse range of mathematical problems.

### Towards Fully Chemical Computation Logic

Based on our current computational approach, using hybrid chemical electronic logic, we can further increase the information processing in the chemical domain and utilize the full potential of massively parallel interactions. This is possible by mapping the energy/cost minimization problem completely into the physical behaviour of the chemical system governed by time evolution. There exists a variety of physicochemical systems capable of spontaneously minimizing energy but it becomes extremely complex to program the system such that mathematical problems can be mapped precisely and solution can be readout. This is a big challenge that exists in molecular computation frameworks. Our computational architecture bypasses this limitation and is capable of utilizing precise digital control for I/O and massively parallel information processing in the chemical domain.

As an example, by increasing the accuracy of the couplings between the cells and finding the input controls to create perfectly symmetric chemical states, the combinatorial optimization problem can be directly mapped to the experimental platform without defining a state machine in the digital domain. Fig. S85 A shows a fully coupled graph with four variables which could be equivalent to 4-number partitioning problem, where each edge defines the couplings coefficients. The direct mapping of this graph to the interconnected chemical oscillator is shown in Fig. S85 B. The four-variables are mapped directly to the chemical states of the active cells. We define four cells to introduce self-interactions and four additional auxiliary cells to complete the connectivity between the diagonally placed spins (x2, x3) and (x1, x4). The cell-cell interface can be activated with the weights proportional to the coupling coefficients. At each step, all neighbouring interactions occur and based on purely chemical decision making, new chemical states should emerge. This emergence of these chemical states can be directly enhanced using digital control, such as PWM states of cell stirrers. By utilizing the natural tendency of the physicochemical system and precise control of couplings using digital control, highly efficient computing hardware can be developed. In this way, complete chemical processing occurs in the chemical domain and digital electronics state machine act as an amplifier to sustain chemical states and precise control of I/O. A viable solution towards a fully chemical computational platform is to switch to electrochemical framework coupled with chemical oscillators, where electrochemical potential or current could be used to precisely define the coupling strength.

Due to mapping of fully connected Hamiltonian on the two-dimensional computational architecture, with the increase in the number of variables and coupling coefficients mapping a large-scale combinatorial optimization problem could be inefficient due to requirement of a large number of auxiliary cells to satisfy all the pairwise interactions such as number partitioning problem on large set or Travelling Salesman Problem with a large number of cities. One simpler solution is to use a network of denser grids such as hexagonal, where each cell can interact up to six neighbours as compared to four neighbour interaction for a square grid. This could lead to more efficient mapping of strongly coupled Hamiltonian and reducing the number of auxiliary cells. Here, we propose an alternative approach to couple cells beyond the nearest neighbours inspired from the idea of implementing high dimensional quasispaces 6 in lower-dimensional geometries. The idea is to miniaturize the computational platform such that the coupling between cells can be instantiated by evolving travelling waves of the reaction-diffusion system. In this case, the coupling between non nearest neighbouring cells can be achieved by programming the path lengths of the interface connecting the cells. By programming the pathlengths in the interconnected network of cells, waves propagating from one cell can reach nearest and next-nearest neighbours at the same time if the path lengths joining them are same. Fig. S86 A shows our current strategy to map Ising spins on a square grid and use auxiliary cells to instantiate interactions between diagonally placed cells. However, based on the proposed strategy, a fully connected four-cell network is achievable without using auxiliary cells as shown in Fig. S86 (B, C). Here, path lengths between S1-S2, S1-S3 and S1-S4 have been increased by creating meander like structures such that all the connections have same path length. In Fig. S86 B, assuming an equilateral triangle (S2-S3-S4) with length L, the path lengths between S1-S2, S1-S2 and S1-S4 should be increased to L.

These mappings can be made more efficient and compact by using multilayer structure as shown in Fig. S86 C, where path lengths between S1-S2, S2-S3, S3-S4 and S4-S1 can be increases to √2 to match the path lengths between diagonally placed elements. Fig. S86 D shows a large scale implementation of the strategy shown in Fig. S86 C to couple next-nearest neighbours. Based on the current approach towards chemical computation together with efficient mappings of non-nearest neighbour couplings, a very efficient computation architecture based on Ising models can be created towards highly efficient computation.

### DISCUSSION

Herein, we present the design, construction, and implementation of a hybrid electronic chemical computer (Deutsch; Lucas) which is built from a programmable chemical array-based around a new type of chemical state machine which is probabilistic in nature. The chemical-computational array operates using the Belousov-Zhabotinsky (BZ) reaction (Horvath et al.; Petrov et al.) which is an oscillating chemical reaction. By using a 3D printed 7×7 cellular array containing the chemical reaction-diffusion medium, it is possible to individually actuate each of the cells using one of the 49 oscillation drivers, and also to program the propagation of the oscillations throughout the array using various combinations of the 84 interface gates allowing communication between the adjacent cells. Using a combination of cell and interface controls, the chemical medium can be monitored so it can be mapped to a digitally representable Chemical State (CS) for each cell, the state of which is read from the analogue to the digital domain. Thus, it creates a hybrid electronic-chemical logic where the digital domain runs deterministic logic using CS and the chemical domain performs analogue computations using chemical oscillations.

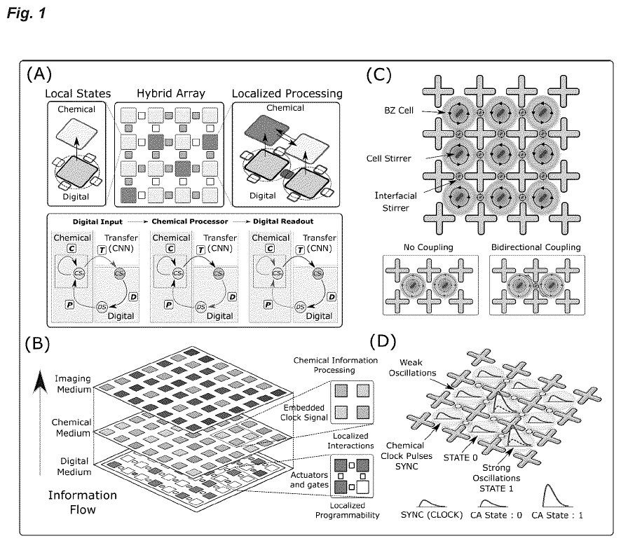

Using the digitally programmable input-output (I/O) system, we showed it is possible to have an error correction system whereby the chemical state can be reinforced, and the effects of phase shift can be eliminated such that the hybrid electronic control system acts to amplify and stabilize the chemically defined states, see FIG. 1. To prove the computation occurs in the chemical substrate, we showed that the system could physically run a cellular automaton (CA), and also that the state space could be expanded way beyond the digital feature space even for simple rules of the elementary CA (Wolfram). The chemical array was also used to two-dimensional CA conveyed as Chemical Cellular Automata (CCA) similar to the emergent behavior as seen in Conway's Game of Life (Gardner) as well as by solving combinatorial optimization problems such as number partitioning (Korf et al.), Boolean satisfiability (Hansen et al.), and the travelling salesman problem (Applegate et al.). These results show that the chemical computer is programmable, scalable, and can tackle a wide range of computation problems traditionally only tractable using digital computers with the potential to be more effective due to the probabilistic nature of the computation.

### Chemical Computing Platform

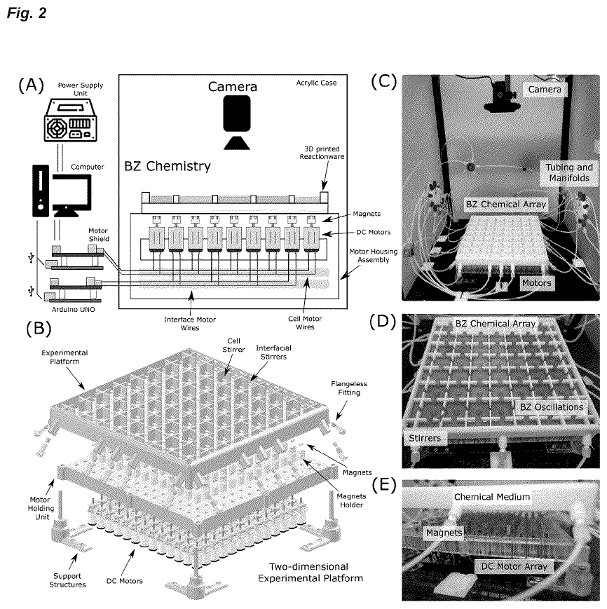

The new chemical array architecture exploits the excitability of the non-linear chemical oscillations of the Belousov Zhabotinsky (BZ) reaction via localized spatial control (Horvath et al.; Petrov et al.). The BZ reaction is highly excitable, can be maintained far from equilibrium, as well as being both spatially and temporally addressable. To exploit these features, we designed an experimental setup where we can program the addressable medium using an electronically controlled input system, see FIG. 1 (A, B). The experimental architecture consists of a 3D printed 1D and 2D grid of interconnected reactors supported on an array of motors equipped with magnetic heads. At the centre of each reactor and the interface of neighbouring cells, magnetic stirrers are placed to match the position of the motor shaft. Each motor is individually addressable, and its speed is controlled using a Pulse Width Modulation (PWM) signal generated using a microcontroller. The schematic diagram and the physical implementation of the two-dimensional experimental setup are shown in FIG. 2. At the start of each experiment, the reagents required to initiate the BZ reaction (solutions of malonic acid, potassium bromate, an iron-based redox catalyst and sulphuric acid) are added into the reactors using an automated liquid handling system interface. The role of the central cell stirrer is to initiate and then maintain the chemical oscillation. It controls the oscillation's amplitude by varying the stirring speed. Cell stirrers also create a vortex flow within the cell constrained by the boundary conditions of the walls of the stirrers. By activating the interfacial stirrer, two vortices of two neighbouring cells can interact with each other through localized mass transfer and mixing at the interface. The mass transfer due to the hydrodynamic coupling between the neighbouring cells leads to interactions between the chemical oscillations of the BZ reactions, see FIG. 1 (C, D). By tuning the speed of the interfacial stirrers (PWM levels), we can control and program the strength of intercellular couplings and limit them to their nearest neighbours. We observed that the chemical oscillations, once created via stirrer actuation, would sustain for a few cycles even after the stirrers were turned off. Thereby demonstrating a short term “memory effect”.

The BZ oscillations induced in a single cell are extremely sensitive to the composition of local redox species and the time of the actuation of the stirrer. This extreme sensitivity on the initial state, localized fluctuations, and time of actuation causes the phase of the chemical oscillations in individual cells to show significant drift with time. We observed that these unfavourable phase shifts between individual cells potentially limited the programmability of the system, and an error correction process to prevent decoherence was needed. To address the potential for errors resulting from state-decoherence in the oscillations, we found that the introduction of a global ‘clock’ signal (SYNC) could be used to ensure the cells remained synchronised. The system-wide SYNC oscillations were created by introducing two actuation operations; firstly, by pulsing the cell stirrers using rectangular waveforms with a long resting time to induce weak oscillations and secondly, by activating the interfacial stirrers to create weak coupling between the nearest neighbours. These global weak oscillations are used for defining clocking signals as well as the low amplitude Chemical State (CS₀). The high amplitude oscillatory states (CS₁) in a cell are created by switching the stirrer from pulsing to continuous mode at a higher stirring rate. These chemical states are the digital representation of the temporal oscillations in the analogue domain with different amplitudes. We have experimentally demonstrated that no significant phase differences occur between the cells on introducing multiple low and high amplitude oscillations, see FIG. 3.

As the BZ reaction proceeds and the malonic acid fuel is consumed, the amplitudes of the oscillations decrease, see FIG. 3 (A, B). To consistently define discrete chemical states based on the observed colour amplitudes, we trained a Convolutional Neural Network (CNN) on a dataset of time-dependent images labelled with discrete states. The three discrete states based on time-dependent colour classification are red (R), light blue (LB) and blue (B) which are also referred to as CNN states. In the presence of only weak oscillations, two distinct CNN states (Red and Light Blue) are recognized, while in the presence of weak and strong oscillations, three distinct states (Red, Light Blue and Blue) emerge, see FIG. 3. A fixed-size temporal window of the CNN states is used to describe the pattern of oscillations, and recognition Finite State Machine (rFSM) logic was used to define the chemical states. In our current design, the rFSM logic consists of two different states: CS₀ and CS₁. The transition to state CS₀ occurs when a weak wave pattern is observed (R→LB→R) and transition to state CS₁ occurs when a stronger oscillation is observed (R→LB→B→LB→R). Using optical readout and precise chemical state recognition, a feedback loop can be completed by implementing a deterministic digital state machine (D) that reads out the chemical states of all or a subset of cells as inputs and returns the PWM levels of the stirrers. These new PWM levels were then applied to the cell and interfacial stirrers (see above).

### One Dimensional Chemical Cellular Automata (1D-CCA)

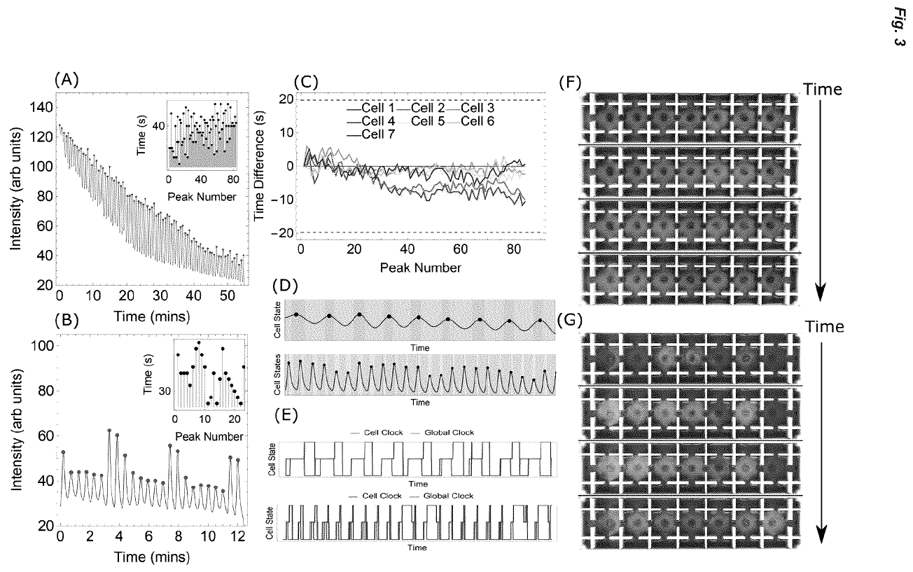

To demonstrate the working principle of the closed-loop hybrid electronic-chemical logic and the programmability with clocking logic, we implemented the Elementary Cellular Automata (CA) rules (Rule 30, 110 and 250) in a fully deterministic way, see FIG. 4 (A&C). In the deterministic model as there is a one-to-one mapping, information loops through digital and chemical domains where the chemical state machine mirrors the digital state machine precisely. However, in this system we demonstrate that the chemical state machine must convey the information for the computation to work. By introducing new automaton rules that exploit the probabilistic computation mode enabled by the high dimensional space associated with the chemical state machine, we are able to explore a larger space of emerging configurations. To do this we defined CA rules where digital state machine (D) reads in chemical states and updates stirrer states (DS) in a deterministic way. Consequently, the new emerging chemical states (defined as Chemical state machine C) are probabilistically controlled due to hydrodynamic interactions of stirrer actuation and previous chemical state, which leads to a new chemical state, see and compare FIG. 4A and B. We then show that simple examples with modified rule 30 with asymmetric actuation on interfacial stirrers leads to significant deviations from one-to-one mapping, see FIG. 4D. Using this approach, we extended the emergent state space by defining the extension of elementary CA towards Chemical Cellular Automata (CCA) which uses deterministic logic in digital and probabilistic in chemical domains. The one-dimensional CCA rules are defined by (Cᵣᵤₗₑ-Iᵣᵤₗₑ) where Cᵣᵤₗₑ reads chemical states and updates cell stirrer digital states and Iᵣᵤₗₑ reads chemical states and updates interfacial stirrer digital states. With two different chemical states (0 and 1), there are 2³=8 possible patterns for the central stirrer and 2²=4 for each of the interfacial stirrer. The total number of possible rules in 1D-CCA are given by 2³2²2²=4096. FIG. 4E shows an example rule table for 1D-CCA rule 30-1 and simulated 1D-CCA rules 30-{1-16} using a basic phenomenological model (see also above).

### Two-Dimensional Chemical Cellular Automata (2D-CCA)

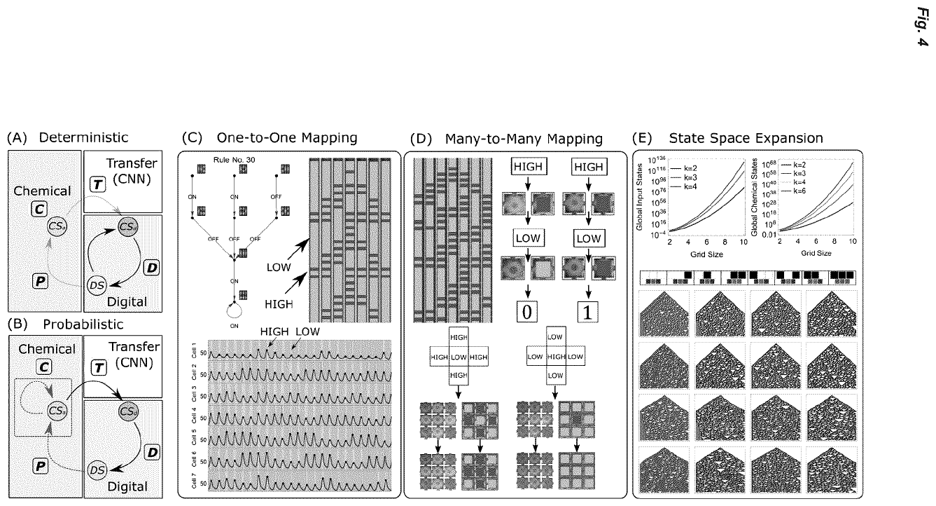

Inspired by the emergent behaviour observed in Conway's Game of Life (Gardner), we extended our CCA in two dimensions to explore the dynamics and emergence of complex patterns. In the 2D-CCA, we defined the basic emergent units as Chemical Entities (Chemits), which are comprised of a combination of five nearest-neighbouring cells. The positions and dynamics of Chemits are defined by the combination of digital states and chemical states. The central core cell showing strong oscillation indicates the existence of the Chemit, and the surrounding neighbours have weak oscillations to manifest the interactions with the environment. As the BZ reaction act as a forced-damped oscillator, we can inject energy into the experimental arena by creating a weak uniform fluctuation field (named as random fluctuations) to create interactions between a Chemit and its surroundings, see FIG. 5(A&B). We then implemented a 2D-CCA digital state machine which takes the emerging chemical states (CS₀/CS₁) from the probabilistic outcomes from the chemical processing and implements 4-state PWM logic with states {S₀,S₁,S₂,S₃}. So corresponds to inactive stirrer state, S₁ introduces the weak fluctuations (weak chemical oscillations on randomly selected cells), S₂ creates the Chemit core and S₃ introduces the interactions between Chemit and surroundings.

The chemical oscillations created by PWM state 1 in the absence of Chemits only leads to the CS₀ chemical state. The Chemit core defined by a high PWM value (S₂) creates high CS₁ states. In the experiment, we observed propagation, replication and competition of Chemits. The emergence of a high chemical state (CS₁) is possible due to the interaction of Chemit cells with local neighbours, and the emergence of a high chemical state at nearest neighbours or next-nearest neighbours which leads to propagation, replication, and competition events. When two Chemits interacts with each other, a competition event happens, and for one Chemit, it has a 50% survival chance. However, in some cases, multiple strong oscillations (CS₁) occur at nearest and next-nearest neighbours, which leads to random selection among multiple events, where a propagation, a replication event or a competition event among all the neighbours was selected randomly. FIG. 5(A, C & D) shows experimental snapshots of propagation and replication events of the Chemit in a 7×7 two-dimensional array and the stirrer states with periodic boundary conditions. The complete description of the 2D-CCA and pseudo-code is described in the SI (see also above). As mentioned previously, the position of the Chemit on the grid is identified using a combination of high chemical state (CS₁) and digital PWM state S₂ of the corresponding stirrer. It is important to demonstrate that the emergent behaviour occurs because of the interaction between the digital state machine and chemical oscillations due to the probabilistic nature of the chemical states (see FIG. 4B), not by direct one-to-one mapping between digital and chemical states. Over a 7×7 array together with periodic boundary conditions, the emergence of peaks in the population of the Chemits due to sudden localized replication events was observed which later fall due to competition events within the constrained space or resource, see FIG. 5 E.

The population and the propagation dynamics of the Chemits are governed by the initial number of Chemits, available space or resource, and random fluctuations. To investigate the emergent behaviour over a larger spatial scale, a chemical probabilistic state machine was developed based on the observed phenomenological model to simulate the dynamics of Chemits behaviour up to 100×100 cell array (see above).

FIG. 6A shows various probabilistic events emerge in Chemits as observed in the experiments and FIG. 6B shows snapshots from the simulations. Similar to the experimental observations, the simulations show the sudden formation of local population clusters due to fast replication and as well as annihilation due to competition events at local clusters of large populations. The steady state population of Chemits show a strong dependence on the size of the cell array independent of the initial population, see FIG. 6C. We further investigated the emergent population and propagation dynamics by changing the number of random fluctuations and cell size (see also above. The steady-state behavior of Chemits at different populations was observed by varying the random fluctuations and cell size, see FIG. 6C (Bottom). As an additional investigation towards 2D-CCA, we explored the phase space by varying parameters describing PWM states corresponding to random fluctuations (S1) and weak interactions (S₃). Steady-state population (after 10000 simulations steps) over the phase space is shown in FIG. 6C (Top Right). The emergent population dynamics and steady-state kinetics are analogous to the population behaviours observed in evolutionary biology which can be further extended towards computation operations.

### Solving Combinatorial Optimization Problems Using Hybrid Computation Machine

Building on the dynamic feedback loop between electronic and chemical states, a hybrid electronic-chemical computing algorithm was implemented to solve quadratic combinatorial optimization problems which can take advantage of the probabilistic chemical state machine to reach the problem solution more efficiently than for a deterministic machine. In the context of Quantum Adiabatic Optimization, various combinatorial optimization problems such as partitioning, satisfiability (SAT) and Hamilton cycles can be formulated as energy/cost minimization problems on an Ising lattice (Lucas; Yue-Guo et al.). Inspired by the Ising or equivalent Quantum Unconstrained Binary Optimization (QUBO) formulations of these problems, we implemented a hybrid electronic-chemical state machine capable of performing energy minimization using chemical states or PWM states equivalent to Ising spin variables. The generalized Hamiltonian up to a quadratic coupling is given by,

$$H ( {Q}_{1} … {Q}_{n} ) = {h}^{( 0 )} + {\sum }_{i = 1}^{n} {h}_{i}^{( 1 )} {Q}_{i} + {\sum }_{1 < i < j < n}^{n} {h}_{ij}^{( 2 )} {Q}_{i} {Q}_{j}$$

where, defines the chemical/PWM state of the cell, h⁽⁰⁾ is an offset energy term, h⁽¹⁾ defines the self-interaction term of the spin equivalent state and h⁽²⁾ defines the coupling between states Qi and Qj. The Ising formulation of the optimization problem can be represented by a connected graph with self-interactions and pairwise couplings for Hamiltonian formulation and mapping to the chemical array). The sign of the coupling coefficients describes the positive (ferromagnetic type) and negative (anti-ferromagnetic type) couplings and the magnitude describes the coupling strength. In the ideal case of computation algorithm in a physical system, when the hybrid electronic chemical system evolves in time, the Ising spin equivalent chemical states should flip according to the interactions defined by local couplings. As such the system can be ergodic and regarded as a Markov chain and should let the Hamiltonian reach the global minimum and the corresponding chemical/PWM states can be interpreted as solutions.

In the current experimental design, it was observed that the perfect symmetric-asymmetric interaction corresponding to the positive and negative couplings between the chemical states is extremely complex to achieve when more than two cells are interacting simultaneously. This is due to the higher probability of occurrence of high chemical state (CS₁) during PWM operations as compared to the lower state (CS₀), as our states are interpreted from oscillation amplitudes. This also makes the temporal evolution of chemical states non-ergodic leading to low efficiency to achieve ground state configuration. To overcome this limitation, we have implemented two different electronic-chemical state machines, where in the first approach the two chemical states (CS₀, CS₁) maps directly to Ising spins (−1, +1) and demonstrates the proof-of-principle computation through the information loop without any neighbouring interactions. Based on the emerging chemical states, the configuration's energy is estimated in-silico and compared with the lowest energy configuration so far and iterated till minimized energy configuration was achieved. In the second hybrid algorithm, we utilized a combination of the greedy algorithm defined in the digital state machine coupled with a probabilistic chemical state machine. In this algorithm, we utilized PWM states of the cell stirrers to map Ising spin variables. We defined probabilistic chemical state machine as “chemical decision making”, which is based on the most probabilistic outcomes of the new chemical states on the application of PWM states on stirrers in the absence of any hysteresis and noise effects. For the pairwise neighbouring interactions, a lookup table on chemical states as shown in FIG. 7A was created and the problem was mapped on the platform (see FIG. 7B). As a result, the emergence of the new chemical states not only depends on PWM states but on the probabilistic properties of the chemical states (see FIG. 7C for computation flowchart). At each step, a comparison was made between ideal states from the lookup table and chemical states, which was utilized for the acceptance of the step towards energy minimization. It becomes evident that the probabilistic outcomes of new chemical states as a result of the combination of analogue and digital processing provide alternate paths towards energy minimization that can be more efficient. The hybrid algorithm does not suffer from the bottleneck of the strong dependence on initial configuration and getting stuck into local minima at the cost of requiring more steps. Hence, a greedy algorithm might be faster in processing but requires sampling over a much larger very large configurational space, whereas a hybrid algorithm might be slower in processing but does not suffer from sampling configurational space leading to efficient information processing. This improvement in efficiency can become more evident with scaling.

We started by mapping variables of the problem to the cells such that all the coupling interactions can be introduced between neighbours. FIG. 7B shows the mapping of a fully connected four-number partitioning problem employing multiple instantiations of the same spins (auxiliary cells) to accomplish pairwise coupling between all the variables of the Hamiltonian. At each step, after flipping the PWM states of the cell randomly, pairwise operations to estimate the energy change were performed using a massively parallel approach. In the chemical decision-making step, if the emergence of the new chemical state is consistent with the lookup table, the energy change was accepted as such else was accepted with a negative sign. The overall energy change was estimated via summation over all the pairwise spins and defined as energy benefit. Based on the net energy benefit for a given step, the flipping was accepted or rejected like the first hybrid logic scheme. Once the energy associated with the current PWM states estimated from the Hamiltonian reaches the global minimum, the solution to the optimization problem is interpreted from the PWM states. FIG. 7D demonstrated the solution to the four-number partitioning problem using the two-hybrid algorithms as described (see FIG. 7E for the second algorithm).

To investigate the influence of chemistry in the hybrid approach, we quantify the capability of our hybrid computational algorithm which utilized a combination of digital greedy algorithm coupled with ergodicity from chemical analogue processing. We formulated an 8-number partitioning problem on the number set S={1,3,4,9,3,5,3,6} and estimated the success probability with all possible starting configuration. We minimized the energy for 100 steps to find the solution at different values of the deterministic index assuming the transition matrix is defined purely by the Markov process. By reduction in the deterministic index (increasing ergodicity by analogue chemical processing), the success probability distribution shrinks, such that there are higher chances to find the solution independent of the initial configuration as compared to the pure deterministic algorithm (see FIG. 7F).

### COMMENTS

We have demonstrated a computational architecture that processes the information in an electronically programmable chemical medium. In this context, it is important to point out that the computation is not digital (i.e. Boolean logic), but a hybrid chemical computation where the computations are done in and take advantage of, the chemical substrate where the input-output (I/O) is achieved via a digital-electronic interface. This can be seen similarly to quantum computing where the Qubits perform the calculation and a digital electronic system is used to both initiate and read out the results of the quantum computation. Our architecture not only can implement elementary cellular automaton rules, but it also has the capacity to compute the emergent behaviour of Chemits resulting from the interaction between electronic and chemical logic. This physical implementation of the cellular automaton could be extended to investigate the interplay between well-defined logic and stochastic effects within the environment towards artificial Chemits.

We also demonstrated two hybrid schemes towards solving combinatorial optimization problems using two Ising spins equivalent states with nearest-neighbouring couplings. Importantly we show that there is a gain in efficiency when the computation is distributed between the digital and the chemical domains, demonstrating that the chemistry is taking an active part in the computation. In addition, the proposed experimental setup is not limited to two-state logic or nearest-neighbouring interactions (local couplings). The experimental design can be modified to introduce fully connected couplings without creating multiple instantiations (auxiliary cells) to improve efficiency by designing equal path lengths between neighbouring and nearest neighbouring cells towards encoding universal computation. This new concept of closed-loop electronic-chemical information processing can be mapped to various chemical-electronic architectures which can be massively scaled using state-of-the-art CMOS electronics. Finally, we think the architecture can be made more powerful by miniaturizing the cells in the array and by replacing the mechanical magnetic stirrers driving the oscillations and programming the chemical states using either photo or electrochemical controls. The external camera could also be replaced with an ion-sensitive array to read out the states. The current challenge is to develop algorithms that can exploit the expanded computational space made accessible via doing the calculations in a chemical substrate. We believe that this work shows a new viable computing paradigm that opens a parallel new route computation different to silicon-based or quantum computation, as well as continue the discussion about the very nature of computation (Moore et al.).

### REFERENCES

All documents mentioned in this specification are incorporated herein by reference in their entirety.
Adamatzky et al. Reaction-Diffusion computers. (Elsevier Inc., 2005)
Applegate et al. The Traveling Salesman Problem: A Computational Study. (Princeton University Press, 2007)
Bennet et al. Nature 404, 247-255 (2000)
Deco et al. Neuron 94, 961-968 (2017)
Deutsch Proc. R. Soc. London A Math. Phys. Eng. Sci. 400, 97-117 (1985)
Fang et al. Sci. Adv. 2, 10.1126/sciadv.1601114 (2016)
Gardner Sci. Am. 223, 120-123. (1970)
Gentil et al. J. Photochem. Photobiol. C Photochem. Rev. 43, 100321 (2020)
Hadaeghi et al. Neurocomputing 338, 233-236 (2019)
Hansen et al. Computing 44, 279-303 (1990)
Horvath et al. Phys. Chem. Chem. Phys., 17, 4664-4677 (2015)
Katsikis et al. Nat. Phys. 11, 588-596 (2015)
Korf Artif. Intell. 106, 181-203 (1998)
Kuhnert et al. Nature 337, 244-247 (1989)
Lin et al. Science 361, 1004-1008 (2018)
Lucas Front. Phys. 2, 10.3389/fphy.2014.00005 (2014)
Moore et al. The Nature of Computation, Oxford University Press (2011)
Parrilla-Gutierrez et al. Nat. Commun. 11, 1442 (2020)
Petrov et al. Nature 361, 240-243 (1993)
Qian et al. Nature 475, 368-372 (2011)
Steinbock et al. Science 267, 868-871 (1995)
Toffoli Int. J. Unconv. Comput. 1, 3-29 (2005)
Torrejon et al. Nature 547, 428-431 (2017)
Tsompanas et al. Sensors Actuators, B Chem. 295, 194-203 (2019)
Watts et al. Nature 393, 440-442 (1998)
WO 2020/058516
Wolfram Commun. Math. Phys 96, 15-57 (1984)
Woods et al. Nature 567, 366-372 (2019)
Yue-Guo et al. Preprint at https://doi.org/10.26434/chemrxiv.10250897 (2019)
Zhu et al. Phys. Rev. Lett. 91, 187902 (2003)

1. A chemical computer comprising a matrix, an input device and an analytical device, wherein:
the matrix comprises a plurality of interconnected reaction spaces holding a reaction mixture, wherein the reaction spaces are interconnected by fluid channels;
the input device is provided to independently address each of a plurality of reaction spaces within the matrix, and to independently address one or more fluid channels; and
the analytical device has a sensor to analyse a reaction characteristic of a reaction mixture in one or more reaction spaces.

2. The chemical computer according to claim 1, wherein the reaction mixture is a reaction mixture for a chemical oscillator reaction.

3. The chemical computer according to claim 2, wherein the chemical oscillator reaction is selected from the group consisting of a Belousov-Zhabotinsky (BZ) reaction, a Briggs-Rauscher reaction and a Bray-Liebhafsky reaction.

4. The chemical computer according to claim 3, wherein the reaction is a Belousov-Zhabotinsky (BZ) reaction.

5. The chemical computer according to claim 1, wherein the reaction mixture is a reaction mixture having a colour change in its reaction, and the analytical device has an optical sensor to analyse the colour change in one or more reaction spaces, optionally wherein there are three or more observable colours in the reaction.

6. The chemical computer according to claim 1, wherein the input device is for independently providing an input to each of a plurality of reaction spaces within the matrix, and for independently providing an input to one or more, such as each of a plurality of, fluid channels within the matrix,
wherein the input is selected from the group consisting of a mechanical force, an optical input, an electrical input, a sonic input, a magnetic input and a thermal input.

7. The chemical computer according to claim 6, wherein the input device is for independently providing a mechanical force to each of a plurality of reaction spaces within the matrix, and for providing a mechanical force to one or more, such as each of a plurality of, fluid channels within the matrix.

8. A computer which comprises a chemical computer according to claim 1.

9. The computer according to claim 8, comprising a plurality of chemical computers, where the chemical computers are provided in series or in parallel.

10. Use of a chemical computer according to claim 1.
